(19) United States
(12) Patent Application Publication
(10) Pub. No.: US 2023/0348378 A1
(43) Pub. Date: Nov. 2, 2023

(54) SMALL MOLECULE ALBUMIN BINDERS

(71) Applicant: Oregon Health & Science University, Portland, OR (US)

(72) Inventors: Dexing ZENG, Portland, OR (US); Lingyi SUN, Portland, OR (US); Zhonghan LI, Portland, OR (US)

(21) Appl. No.: 18/042,209

(22) PCT Filed: Aug. 20, 2021
(86) PCT No.: PCT/US2021/047024

§ 371 Date: Feb. 17, 2023

Related U.S. Application Data
(60) Provisional application No. 63/068,259, filed on Aug. 20, 2020.

(51) Int. Cl. C07D 207/46; A61K 51/04; A61K 51/08

(52) U.S. Cl. CPC C07D 207/46; A61K 51/0497; A61K 51/082; A61K 2121/00

(57) ABSTRACT

Compounds are described having albumin binding groups, where said compounds can be complexed with therapeutic and/or diagnostic agents and further can include targeting functionality. When introduced into the circulatory system the compounds and complexes bind serum albumin, and thereby exhibit useful properties including enhanced circulatory half-life, improved uptake by target tissues, and improved target/nontarget ratios. These properties make the compounds and complexes useful in therapeutic and diagnostic methods.

### RELATED APPLICATIONS

This application claims priority to U.S. Provisional Patent Application No. 63/068,259, filed on Aug. 20, 2020, entitled “SMALL MOLECULE ALBUMIN BINDERS,” which is hereby incorporated herein by reference in its entirety.

### TECHNICAL FIELD

The present disclosure concerns small molecules useful as albumin binders that may be used in diagnostic and pharmaceutical applications.

### BACKGROUND OF THE INVENTION

Human serum albumin is the most predominant plasma protein in the blood stream, and it accounts for approximately 55-60% of the total serum proteins. With this incredible abundance, albumin performs many physiological functions: maintenance of oncotic pressure within vascular system, essential multi-carrier of many hydrophobic endogenous and exogenous moieties, such as lipids, metal ions, hormones, amino acids and some biomedical drugs. Because the size of albumin protein (˜66.5 kDa) is over the threshold of renal ultrafiltration, it cannot pass through the pores in the glomerular membranes and thus is retained in the blood. In addition, albumin protein exhibits a unique interaction with neonatal Fc receptor (FcRn), and thus is excluded from cellular catabolism via recycling and transcytosis pathways. These features give albumin protein a relatively long serum half-life time (approx. 19 days). Because of excellent serum stability and long serum half-life time, albumin has been utilized for drug delivery either by direct genetic fusion, or covalent bonded conjugation, or by non-covalent binding interaction, to render favorable pharmacokinetics and pharmacodynamics.

Via binding with albumin protein, pharmaceuticals with incorporated albumin-binder can achieve significantly enhanced tumor uptake, which can be attributed to three major reasons: 1) reversible binding to albumin protein can prolong serum half-life time; 2) the relatively big size of albumin-pharmaceutical complexes provides an enhanced permeation and retention (EPR) effect, and 3) albumin and albumin-pharmaceutical complex can play a nutritional source role for tumor growth. Various reversible albumin-binding molecules (e.g., ABI)] have been incorporated into radioligands for nuclear imaging and/or radionuclide therapy. As shown in FIG. 1, one example of an albumin binder-radioligand conjugate can comprise three components: a cancer biomarker-specific ligand for tumor targeting, a radionuclide for imaging and/or radionuclide therapy, and an albumin-binder for albumin protein binding to enhance tumor uptake. Unlike the high binding affinity (e.g., <50 nmol) between radioligand and tumor-receptor, a reversible albumin-binder having moderate binding affinity (e.g., in μM level) to albumin protein can easily disassociate from albumin protein, and then accumulate in the tumor due to much stronger binding with the targeted tumor-receptor.

Heretofore, two classes of portable, low molecular weight and reversible albumin binders have been investigated for nuclear imaging and radioligand therapy. One type of portable, low molecular weight and reversible albumin binder is based on 4-(p-iodophenyl) butyric acid, and it binds with Sudlow binding sites II of albumin protein. The other type is truncated from Evans Blue (EB), and it binds with Sudlow binding sites I of albumin protein. Both ABI and EB albumin binders exhibit moderate binding to albumin protein, with affinity of 3.2 μM (K_{d} of ABI) and 2.5 μM (K_{d} of EB). ABI and EB incorporated radioligands have showed prolonged blood circulation and improved tumor uptake, when compared to the corresponding radioligand that does not contain albumin binder. Despite exhibiting enhanced tumor uptake and prolonged tumor retention, incorporating of ABI (or) EB may also result in considerable concerns on reduced tumor/nontumor ratios, due to much higher uptake on normal tissue (such as, blood, bone marrow, kidneys, etc.). Therefore, a portable albumin binder that can enhance tumor uptake and also improve tumor/non-tumor ratios is still highly desirable for nuclear imaging and radionuclide therapy.

### BRIEF DESCRIPTION OF THE DRAWINGS

FIG. 1 provides an illustration of the proposed mechanism of albumin binder incorporated radioligands.

FIG. 2 provides a structure of an albumin binder incorporated into a binder-drug conjugate, illustrating albumin binding moieties.

FIG. 3 provides a structure of an example conjugate of an albumin binder, a radionuclide chelator (DOTA), and a targeting ligand (RGD) according to an embodiment.

FIGS. 4A-4D show examples of alternative structures for an albumin binder-radionuclide chelator conjugate, in which FIG. 4A incorporates S-lysine; FIG. 4B incorporates R-lysine; FIG. 4C includes an added amide linkage; and FIG. 4D features a shift in amide linkage position.

FIG. 5 represents an in vitro binding assessment of a selection of albumin binder-chelator conjugates with human serum albumin proteins.

FIG. 6 represents an in vitro binding assessment of ⁶⁴Cu-DOTA-SFLAP3-PEG4-ABCF3 (abbreviated as SFLAP3-ABCF3), compared to ⁶⁴Cu-DOTA-SFLAP3 (abbreviated as SFLAP3).

FIGS. 7A and 7B represent an evaluation of ¹¹¹In labeled RGD-ABCF3, RGD and RGD-ABI in mice bearing BxPC3 xenograft: (FIG. 7A) biodistribution, and (FIG. 7B) tumor/non-tumor ratios (mean±SD).

FIGS. 8A and 8B represent an evaluation of ¹¹¹In labeled RGD-ABCF3, RGD and RGD-ABI in mice bearing CT26 xenograft: (FIG. 8A) biodistribution, and (FIG. 8B) tumor/non-tumor ratios (mean±SD).

FIGS. 9A and 9B represent an evaluation comparing ⁶⁴Cu labeled PSMA617-ABCF3 and PSMA617 in mice bearing PSMA⁺ PC3pip and PSMA-PC3 xenograft: (FIG. 9A) biodistribution, and (FIG. 9B) tumor/nontumor ratios (mean±SD).

FIGS. 10A and 10B represent an evaluation of ¹¹¹In labeled FRGD-ABCF3, and FRGD in mice bearing BXPC3 xenograft: (FIG. 10A) biodistribution, and (FIG. 10B) tumor/non-tumor ratios. (mean±SD).

FIGS. 11A and 11B represent an evaluation comparing in vivo performance of SFLAP3 incorporated with various albumin binders in mice bearing BxPC3 xenograft (24 h): (FIG. 11A) biodistribution, and (FIG. 11B) tumor/nontumor ratios. (mean±SD).

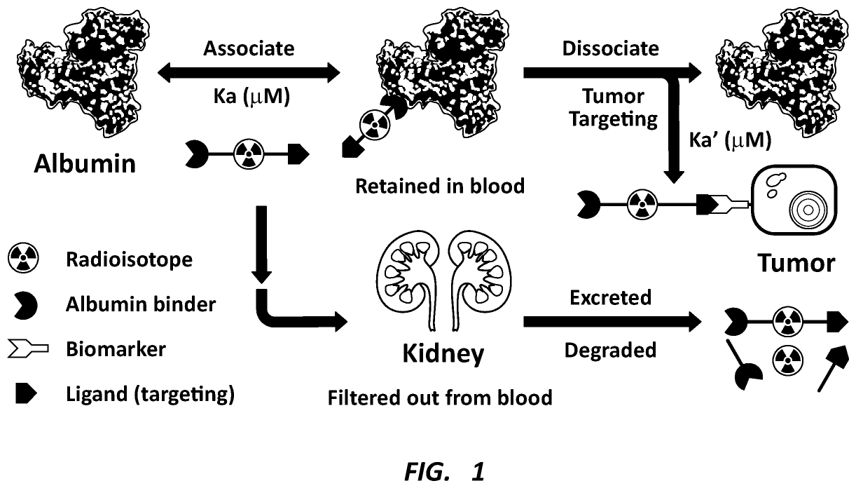

### DETAILED DESCRIPTION OF THE INVENTION

In one embodiment of the invention is provided a compound of Formula (I):

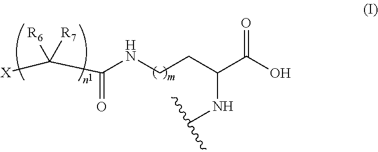

wherein:
X is selected from the group of:

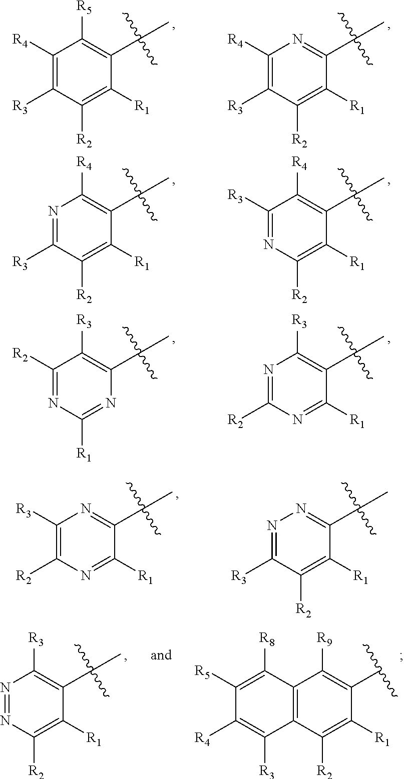

R₁, R₂, R₃, R₄, R₅, and when present, R₈ and R₉ are each independently selected from the group of H, F, Cl, Br, I, SF₃, SF₂Cl, SF₅, SF₄Cl, C₁-C₆ straight or branched alkyl, C₁-C₆ straight or branched fluoroalkyl (including CF₃), C₁-C₆ straight or branched fluorinated alkoxy, and isotopes thereof, or a substituent selected from the group of:

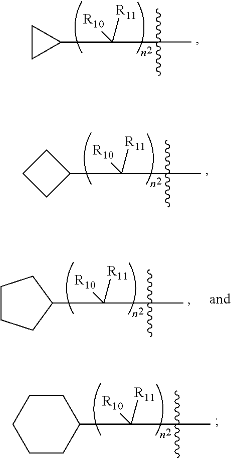

R₆ and R₇ are in each instance independently selected from H and F, and isotopes thereof, or combined in an oxo group;
R₁₀ and R₁₁ are in each instance independently selected from H and F;
n¹ is an integer selected from the group of 1, 2, 3, 4, 5, and 6;
n² is an integer selected from the group of 1, 2, 3, and 4; and
m is an integer selected from the group of 1, 2, 3, 4, 5, 6, 7, and 8; with the proviso that when X is a substituted benzyl group at least one of R₁, R₂, R₃, R₄, and R₅ is selected from the group of F, SF₃, SF₅, and C₁-C₆ straight or branched fluoroalkyl (including CF₃) or a substituent selected from the group of:

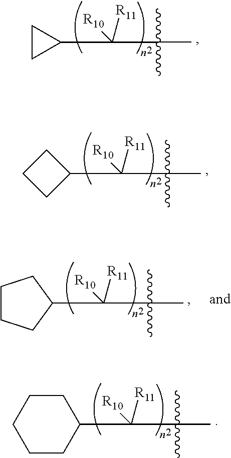

In some embodiments, Formula (I) is further defined with the proviso that not more than 3 of R₁, R₂, R₃, R₄, R₅, and when present, R₈ and R₉ is a moiety selected from the group of C₁-C₆ straight or branched fluoroalkyl (including CF₃), SF₂, SF₂Cl, SF₅, and SF₄Cl. In some embodiments, Formula (I) is further defined with the proviso that, when present, at least one R₁₀ group must be F or ¹⁸F.

The albumin binder as shown above can be described as comprising an albumin binding component (also referred to generally herein by the identifier “ABX”) that includes a carboxylic acid having a chain length n¹ and a substituent binding group X; and a spacer, such as an amino acid with a side chain of length m+1. In some embodiments, the albumin binder is a selected enantiomer of a chiral compound with the central carbon of the spacer as a chiral center. Non-limiting examples of the albumin binding component are as follows:

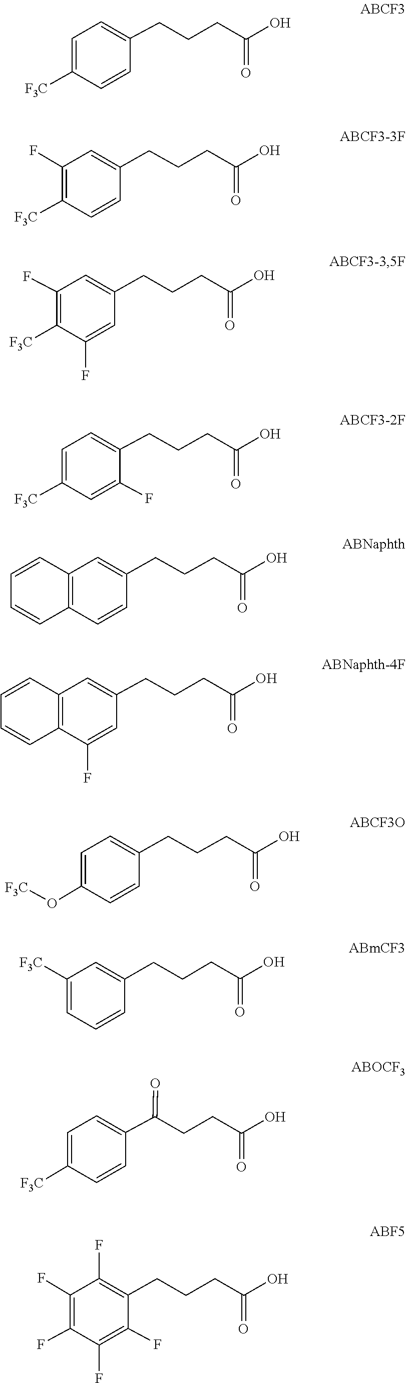

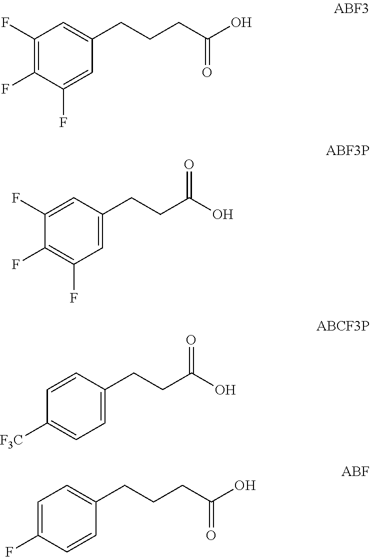

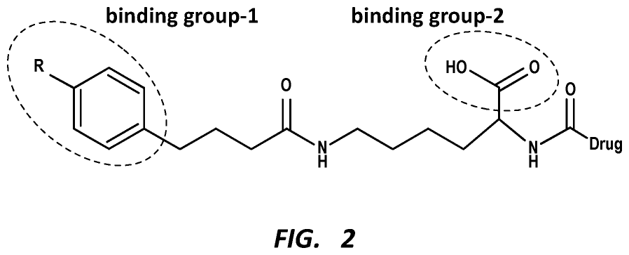

An aspect of the present disclosure is that the compounds described herein bind serum albumin. More specifically, in various embodiments such binding occurs through non-covalent interaction between one or more moieties represented in Formula (I) and a binding site on the albumin protein. As shown in FIG. 2, one such binding group (Binding Group 1) can be a substituted aromatic group represented by X in Formula (I). Another such binding group (Binding Group 2) can be the carboxyl group of the spacer as shown in Formula (I). While the present disclosure is not bound to one particular theory, it is believed that binding can be achieved by one or both of a) interaction of Binding Group 1 with a hydrophilic pocket of the albumin protein, and b) interaction of Binding Group 2 with a hydrophobic pocket of the albumin protein.

By virtue of their albumin binding properties in combination with the kinetics of serum albumin in vascular circulation, it is contemplated that various compounds described herein can be complexed with one or more chemical entities of therapeutic and/or diagnostic interest, where the therapeutic or diagnostic effect of said entities can be enhanced by binding of the complex to serum albumin. In aspect, some such enhancements arise from the tendency of albumin to concentrate in the tumor microenvironment and other tissues affected by diseases such as inflammatory diseases. In some embodiments, a composition comprises a product of a reaction in which a physiologically active molecule such as a therapeutic agent is complexed with the albumin binding compounds described herein. The product of such a reaction may be interchangeably referred to herein as a “complex” or “conjugate” and methods of forming them may be interchangeably referred to herein as “complexing” or “conjugating”/“conjugation”. As used herein, the terms “complex” and “conjugate” each refer to a molecule comprising two different molecules combined by a covalent or noncovalent bond. In some embodiments, a complex or conjugate may comprise two or more physiologically active molecules covalently bonded through a linking molecule. In some embodiments, one of the physiologically active molecules may comprise a targeting ligand. The term “targeting moiety” or “targeting ligand” refers to any molecule that provides an enhanced affinity for a selected target, e.g., a cell, cell type, tissue, organ, region of the body, or a compartment, e.g., a cellular, tissue or organ compartment. The targeting moiety or ligand can comprise a wide variety of entities, including naturally occurring molecules, or recombinant or synthetic molecules. Targeting moieties or targeting ligands include, but are not limited to, antibodies, antigen binding fragments of antibodies, antigens, folates, EGF, albumin, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

In some embodiments, the therapeutic agent is a drug, such as an anti-cancer drug or a drug against an inflammatory disease. In certain embodiments, the drug is an anti-cancer drug, and more particularly can be one of following: alkylating drugs, anthracyclines, cytotoxic antibiotics, anti-metabolites, vinca alkaloids, platinum-based anti-neoplastic agents, taxanes, epothilones, histone deacetylase inhibitors, topoisomerase I inhibitors, topoisomerase II inhibitors, kinase inhibitors, retinoids, nucleotide analogs, and precursor analogs.

In various embodiments, the anti-cancer drug is selected from actinomycin, all-trans retinoic acid, alitretinoin, azacytidine, azathioprine, bexarotene, leomycin, bortezomib, carboplatin, capecitabine, cisplatin, chlorambucil, cyclophosphamide, cytarabine, daunorubicin, dacarbazine, docetaxel, doxifluridine, doxorubicin, epirubicin, epothilone, erlotnib, etoposide, fluorouracil, gefitnib, gemcitabine, hydroxyurea, idarubicin, imatinib, irinotecan, mechlorethamine, mercaptopurine, methotrexate, melphalan, mitoxantrone, nitrosourea, oxaliplatin, paclitaxel, pemetrexed, tafluposide, temozolomide, teniposide, tioguanine, topotecan, tretinoin, valrubicin, vemurafenib, vinblastine, vincristine, vindesine, vinorelbine, vismodegib, vorinostat, cyclophosphamide, ifospfamide, busulfan, lomustine, carmustine, chlormethine, altretamine, estramustine, treosulfan, thiotepa, mitobronitol, aclarubicin, idarubicin, dactinomycin, mitomycin, pentostatin, fludarabine, cladribine, raltitrexed, tegafur, amsacrine, asparaginase, trastuzumab, and derivatives thereof.

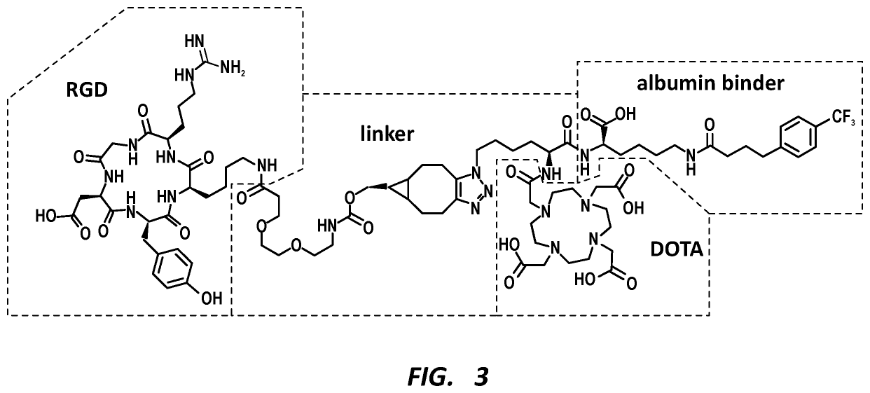

Also provided are conjugate compounds, a nonlimiting example of which is shown in FIG. 3. Such conjugates can comprise the covalently or non-covalently bonded units of:
a) an albumin binding compound (e.g., ABCF3 as shown in FIG. 3);
b) a linker; and
c) one or more functional groups effective for therapy and/or imaging.

As illustrated in FIG. 3, such functional groups may include one or both of:
i. a chelator agent (e.g., DOTA as shown in FIG. 3); and
ii. a targeting ligand (e.g., RGD as shown in FIG. 3).

Some applications of the compounds described herein, e.g. imaging, theranostic, radiotherapeutic and targeted drug delivery applications, may benefit from additional targeting functionality. In some embodiments, a composition comprises a product of a reaction in which a targeting ligand is complexed with the albumin binding compounds described herein. In some embodiments the targeting ligand is non-covalently bound to the albumin binding compound. In other embodiments, the targeting ligand is covalently linked to the albumin binding compound, and optionally said covalent binding is through a linker selected to provide a certain level of performance in diagnostic effect, therapeutic effect and pharmacokinetics of the ligand or composition as a whole. The targeting ligand can be a molecule of any type that is amenable to incorporation into the compound by known methods, including proteins, polysaccharides, nucleic acids, peptides, aptamers, and small molecules.

In various embodiments, the targeting ligand is targeted to a receptor that is overexpressed in a tissue affected by a disease. In certain embodiments, the receptor is a molecule expressed in tumor cells or cells in inflamed tissues, including but not limited to integrins, lectins, and cytokines. In particular embodiments, the receptor is an integrin, such as, but not limited to, α_{V}β₃, α_{V}β₄, α_{V}β₅, α_{V}β₆, or α₅β₁. In other embodiments, the targeting ligand is targeted to a drug or a metabolite of a drug, such that the compound will co-locate with, and optionally bind to, the drug in a subject when both are administered within a certain timeframe. In some embodiments, the targeting ligand may be an inhibitor of the target receptor, e.g., receptor tyrosine kinases such as EGFR and HER2. In some embodiments the targeting ligand comprises a targeted nanobody.

Peptides used as targeting ligands can comprise linear, branched, or cyclic peptides known for such uses. Non-limiting examples of peptides for these uses include arginylglycyclaspartic acid (RGD), galacto-RGD₂, P-RGD, RGD₂, P-RGD₂, 2G-RGD₂, 2P-RGD₂, 3G-RGD₂, 3P-RGD₂, 3P-RGK₂, RGD₄, 6G-RGD₄, and 6P-RGD₄, as described by Shi et al., Biophys Rep 2016, 2(1):1-20, the contents of which are incorporated herein by reference in their entirety, as well as FRGD, SFLAP3, FAPI, AE105, NT20.3, A20FMDV2, Pentixafor, JR11, DOTATATE and PSMA-617.

Vitronectin, osteopontin, fibrinogen, and fibronectin serve as natural ligands for α_{V}β₁, α_{V}β₃, α_{V}β₅, α_{V}β₆, α_{V}β₈, α₅β₁, α8β₁, α_{IIb}β₃ integrins (RGD recognition sequence). Fibronectin, vascular cell adhesion molecule 1, mucosal addressin cell adhesion molecule 1, and intercellular cell adhesion molecule 1 serve as natural ligands for α₄β₁, α₉β₁, α₄β₇, α_{E}β₂, α_{L}β₂, α_{M}β₂, α_{X}β₂, and α_{D}β₂ integrins (LDV and related sequences). Collagen and laminin serve as natural ligands for α₁β₁, α₂β₁, α₁₀β₁, and α₁₁β₁ integrins (GFOGER recognition sequence). Laminin also serves as a natural ligand for α₃β₁, α₆β₁, α₇β₁, and α₆β₄ integrins.

In some embodiments, the complex includes a radionuclide chelator to provide for radiodiagnostic or radiotherapeutic applications when chelated to a radionuclide. In various embodiments, the chelator is one of any known to be capable to chelate radionuclide metals suitable for these uses, such radionuclides including but not limited to ¹⁷⁷Lu, ⁸⁶Y, ⁸⁹Zr, ⁴⁷Sc, ⁴⁴Sc, ²¹³Bi, ^{99m}Tc, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁴⁸Gd, ⁵⁵Fe, ²²⁵Ac, ²¹²Bi, ²¹¹At, ⁴⁵Ti, ⁶⁰Cu, ⁶¹Cu, ⁶⁷Cu, and ⁶⁴Cu. Such chelators include 2,2′,2,2″′-(1,4,7,10-tetraazacyclododecane-1,4,7,10-tetrayl)tetraacetic acid (DOTA), Hexahydro-1H-1,4,7-triazonine-1,4,7-triacetic acid (NOTA), 1,4,7-Tris(phosphonomethyl)-1,4,7-triazacyclononane (NOTP), ((1,4,7-triazonane-1,4,7-triyl)tris(methylene))tris(phosphinic acid) (TRAP), N′-[5-[[4-[[5-(acetylhydroxyamino)pentyl]amino]-1,4-dioxobutyl]hydroxyamino]pentyl]-N-(5-aminopentyl)-N-hydroxy-butanediamide (DFO), 2,2′,2″,2″′-((((carboxymethyl)azanediyl)bis(ethane-2,1-diyl))bis(azanetriyl))tetraacetic acid (DTPA), 3,12-bis(carboxymethyl)-6,9-dioxa-3,12-diazatetradecanedioic acid (EGTA), 2,2′,2″,2″′-(ethane-1,2-diylbis(azanetriyl))tetraacetic acid (EDTA), 7-[2-[bis(carboxymethyl)amino]-3-(4-nitrophenyl)propyl]hexahydro-1H-1,4,7-Triazonine-1,4(5H)-diacetic acid (C-NETA), 2-(4,7-bis(carboxymethyl)-1,4,7-triazonan-1-yl)pentanedioic acid (NODAGA), 2-(4,7,10-tris(carboxymethyl)-1,4,7,10-tetraazacyclododecan-1-yl)-pentanedioic acid (DOTAGA), 1,4,7-triazacydononane-1-[methyl(2-carboxyethyl)-phosphinic acid]-4,7-bis[methyl(2-hydroxymethyl)phosphinic acid] (NOPO), 3,6,9,15-tetraazabicyclo[9,3,1]pentadeca-1 (15), 11,13-triene-3,6,9-triacetic acid (PCTA), N,N″-bis[2-hydroxy-5-(carboxyethyl)-benzyl]ethylenediamine-N,N″-diacetic acid (HBED-CC), N,N′-bis(2,2-dimethyl-2-mercaptoethyl)ethylenediamine-N,N′-diacetic acid (6SS), 1-(4-carboxymethoxybenzyl)-N-N′-bis[(2-mercapto-2,2-dimethyl)ethyl]-1,2-ethylenediamine-N,N′-diacetic acid (B6SS), N,N′-dipyridoxylethylenediamine-N,N′-diacetic acid (PLED), 1,1,1-Tris-(aminomethyl)ethane (TAME), nitrilotrimethylphosphonic acid (NTP), 2,2′,2″,2″′-(1,4,8,11-tetraazacyclotetradecane-1,4,8,11-tetrayl)tetraacetic acid (TOTA), and 2-BAPEN, and derivatives thereof.

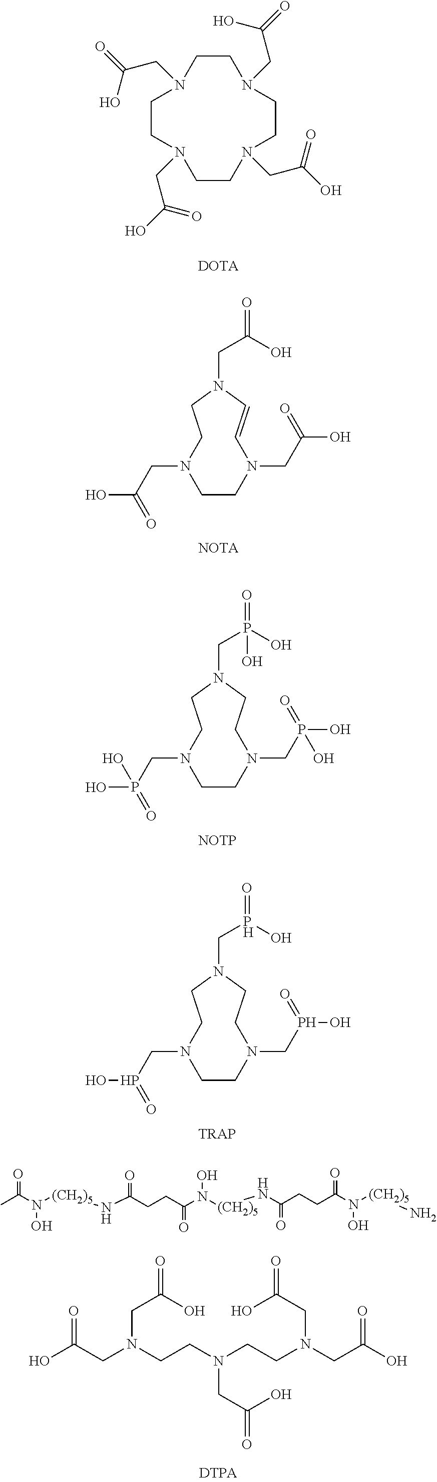

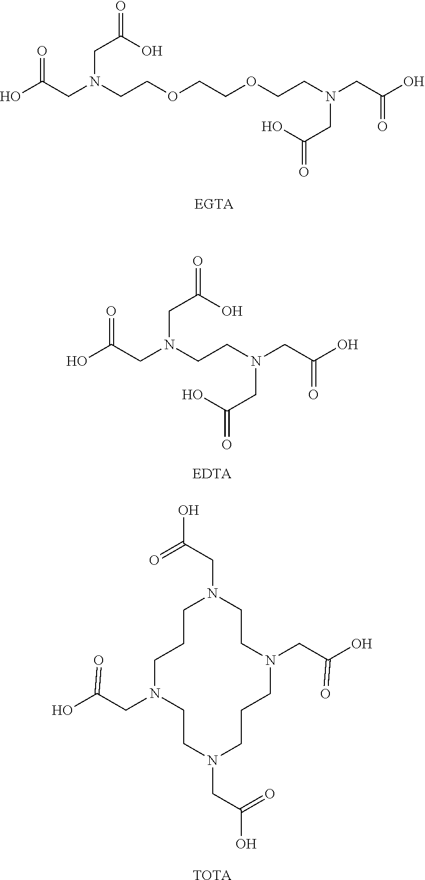 [SMILES: O=C(O)CN(CCOCCOCCN(CC(=O)O)CC(=O)O)CC(=O)O ; O=C(O)CN(CCN(CC(=O)O)CC(=O)O)CC(=O)O ; O=C(O)CN1CCCN(CC(=O)O)CCN(CC(=O)O)CCCN(CC(=O)O)CC1 ; "TOTA"]

The radionuclide used in the complexes herein may also be selected from the non-limiting groups of:
a) radioisotopes useful in radiopharmaceutical imaging, such as positron-emitting radioisotopes used in positron emission tomography (PET), including ¹⁸F, ¹¹C ¹³N, ⁷⁵Br, ⁷⁶Br, ¹²⁴I, ⁶⁴Cu, ⁴⁸V, ⁵²Fe, ⁵⁵Co, ⁸²Rb, ^{94m}Tc, ¹³³Xe, or ⁶⁸Ga, and gamma-emitting radioisotopes used in single photon emission computed tomography (SPECT) scanning, including ^{99m}Tc, ¹²³I, ¹²⁵I, ¹²³I, ¹³¹In, ^{113m}In, ¹⁵O, ²⁰¹Tl, ⁶⁷Cu or ⁶⁷Ga;
b) radioisotopes include therapeutic isotopes used for cancer cell destruction and pain treatment in palliative care for bone cancer or arthritis, including ¹³¹I, ⁹⁰Y, ¹⁸⁸Rh, ¹⁷⁷Lu, ⁴⁷Ca, ¹⁶⁹Er, ³²P, ²²³Ra, ²¹²Pb, and ⁸⁹Sr; and
c) radioisotopes used for testing and diagnostic purposes, including ¹⁴C, ⁵¹Cr, ⁵⁷Co, ⁵⁸Co, ³H, ⁵⁹Fe, ^{81m}Kr, ²²Na, and ²⁴Na.

The albumin binding compound can also incorporate radionuclides according to some embodiments. Some embodiments provide a compound of Formula (I), wherein at least one variable selected from the group of R₁, R₂, R₃, R₄, R₅, R₆, R₇, R₈, R₉, R₁₀, and R₁₁, comprises ¹⁸F or an ¹⁸F-containing functional group, such as CF₂¹⁸F.

The linker can comprise any structure selected to provide a certain level of performance in diagnostic effect, therapeutic effect and pharmacokinetics of the ligand or composition as a whole. In various embodiments, the linker may include structural motifs directed to this function, for example, polyether linkages including, but not limited to, polyethylene glycol. In some embodiments, the linker comprises the structure of Formula (L1), below, wherein n₁ is an integer selected from the group of 1-30 and n₂ is an integer selected from the group of 2-10. In other embodiments, the linker group is comprised of a compound of Formula (L1), below, wherein n₁ is an integer selected from the group of 2-20 and n₂ is an integer selected from the group of 2-8. In further embodiments, the linker group is comprised of a compound of Formula (L1), below, wherein n₁ is an integer selected from the group of 2-15 and n₂ is an integer selected from the group of 2-6.

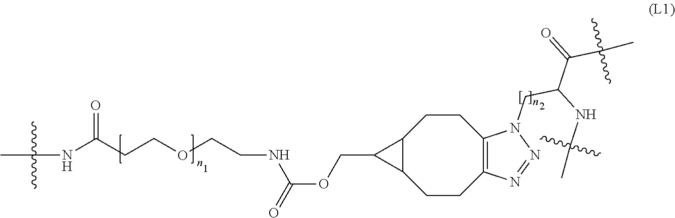

In additional embodiments, the linker comprises the structure of Formula (L2), below, wherein n₁ is an integer selected from the group of 1-30, n₂ is an integer selected from the group of 2-10, and n₃ is an integer selected from the group of 1-10. In other embodiments, the linker group is comprised of a compound of Formula (L2), below, wherein n₁ is an integer selected from the group of 2-20, n₂ is an integer selected from the group of 2-8, and n₃ is an integer selected from the group of 2-8. In further embodiments, the linker group is comprised of a compound of Formula (L2), below, wherein n₁ is an integer selected from the group of 2-15, n₂ is an integer selected from the group of 2-6, and n₃ is an integer selected from the group of 2-6.

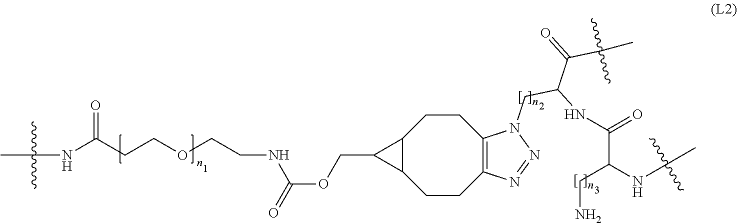

The present disclosure also encompasses various methods and uses involving the compounds described herein. In an aspect, the circulatory half-life of the compounds described herein is enhanced by their ability to bind serum albumin due to the long-circulating nature of albumin. With respect to functional entities complexed to albumin binding compounds in various embodiments, this allows those entities to exert their effect for a longer period of time post-administration, particularly where the functional group as a separate entity, e.g. a standalone drug, would normally be rapidly cleared if administered alone. Accordingly, a method of increasing the circulatory half-life of a drug can comprise complexing the drug with one of the compounds described herein.

Another aspect is provided by incorporation of these compounds into a complex having targeting properties in combination with reversible binding to albumin. That is, such a composition introduced into the vascular circulation will bind to albumin in the blood until the albumin reaches tissues expressing the target receptor, upon which the targeting moiety's affinity for its target can cause the compound to dissociate from the albumin and concentrate in the target location. In various embodiments, the binding affinity of the targeting ligand for the corresponding target is greater than the binding affinity of any part of the compound—or more particularly constituent X—for albumin. Thus a smaller dose of the functional group entity may be required to produce a particular effect than if the entity were administered alone. Accordingly, in some embodiments, a method of increasing the therapeutic efficacy of a drug can comprise complexing the drug with one of the compounds described herein.

Further, higher rates of conjugate uptake in the target compared to other organs can make it easier to attain effective dosing with reduced risk of toxicity to those organs. For example, high renal uptake is a significant concern in radioligand therapy, since kidneys have been often considered as the dose limiting organ in this application. One of the important benefits that the albumin binders described herein can offer is to improve tumor/nontumor (e.g., tumor/kidney) ratios compared to those without albumin binder; in contrast, some previously reported albumin binders normally resulted in reduced tumor/nontumor (e.g., tumor/kidney) ratios, and consequently bring concerns to their applications in: 1) molecular imaging when the contrast is significantly reduced by high nontumor uptake(s) in an adjacent tissue, and also 2) radioligand therapy if the radiotoxicity to an tissue with high nontumor uptake is not very tolerable.

In some embodiments, a method of treating a subject comprises administering a therapeutically effective dose of a composition comprising an albumin binding compound complexed with a therapeutic agent as described herein. In particular embodiments the therapeutically effective dose is lower than a minimum therapeutically effective dose of the drug alone. In some embodiments, a method of treating a subject comprises administering a composition comprising a targeting ligand that includes a radionuclide chelator to the subject and measuring a level of the composition in a sample from the subject. Administration may be accomplished through any route that will bring the compound into contact with serum albumin. In various embodiments, the compound is administered intravenously or intramuscularly.

The compounds and compositions described herein may be prepared by methods known in the art, with the use of many commercially available materials. In one method, useful intermediates may be prepared by stirring a solution of substituted X-group-terminated butanoic acid (1) and EDC·HCl in CH2Cl2, followed by addition of 1-hydroxypyrrolidine-2,5-dione and triethylamine (TEA). The product may then be washed with saturated NCI solution and dried over MgSO₄.

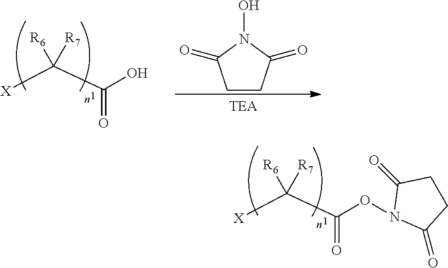

Also provided herein is a compound of the Formula (II):

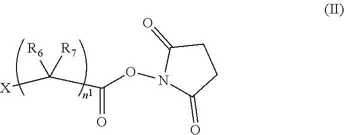

wherein:
X is selected from the group of:

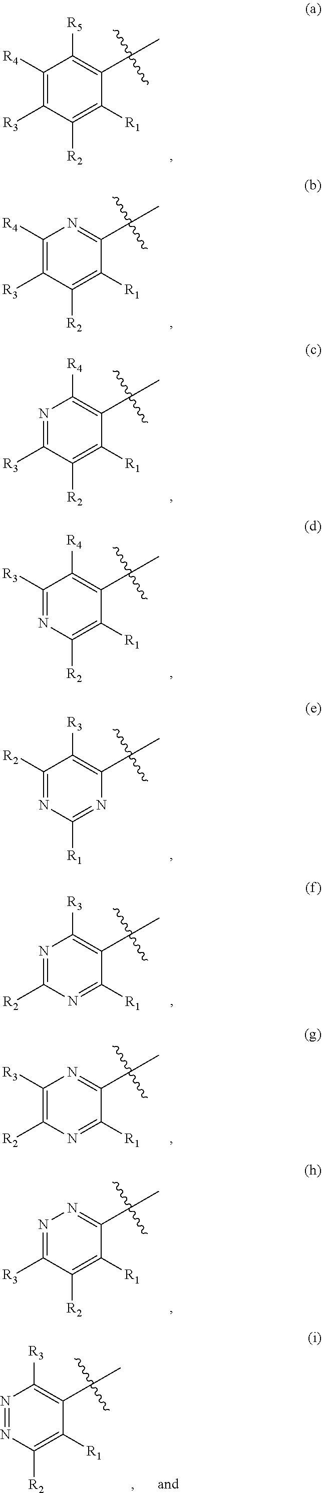

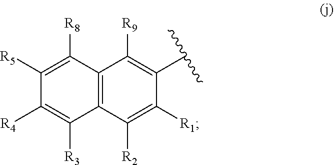

R₁, R₂, R₃, R₄, R₅, R₈ and R₉ are each independently selected from the group of H, F, Cl, Br, I, SF₃, SF₂Cl, SF₅, SF₄Cl, C₁-C₆ straight or branched alkyl, C₁-C₆ straight or branched fluoroalkyl (such as, for example, CF₃), C₁-C₆ straight or branched fluorinated alkoxy, or a substituent selected from the group of:

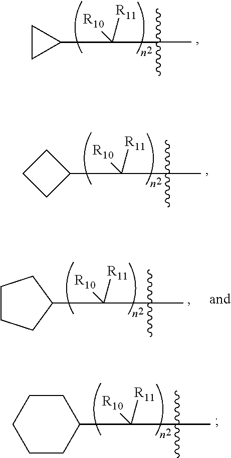

R₆ and R₇ are in each instance independently selected from H and F, and isotopes thereof, or combined in an oxo group;
R₁₀ and R₁₁ are in each instance independently selected from H and F, with the proviso that, when present, at least one R₁₀ group must be F;
n¹ is an integer selected from the group of 1, 2, 3, 4, 5, and 6; and
n² is an integer selected from the group of 1, 2, 3, and 4.

It is understood that additional separate groups of compounds are provided for each “X” group (a) through (j) in Formula (II), as shown below. In each of Formulas (IIa) to (IIj) all variables, including R₁, R₂, R₃, R₄, R₅, R₆, R₇, R₈, R₉, R₁₀, R₁₁, n¹, and n², are as defined for Formula (II).

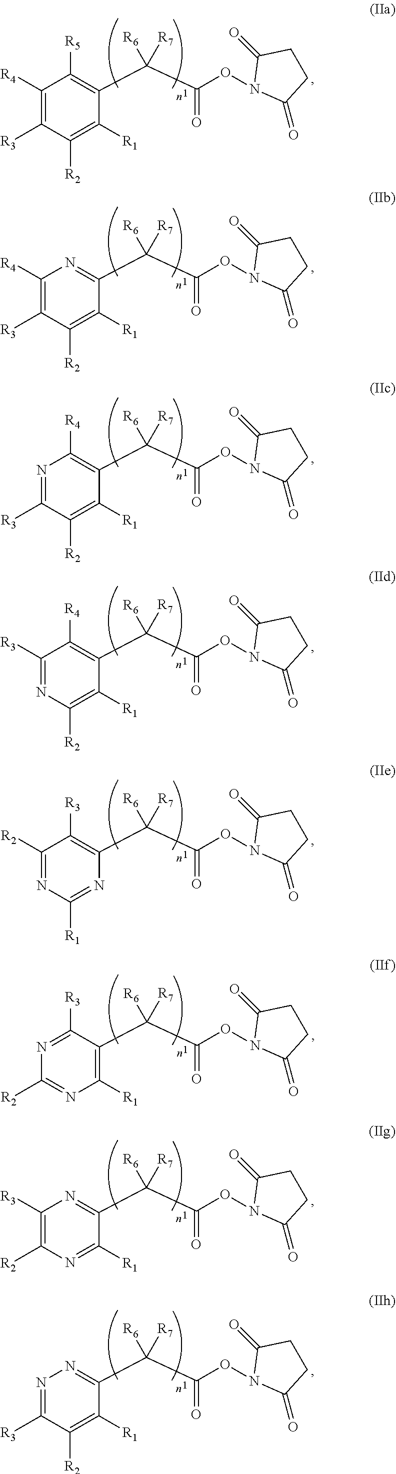

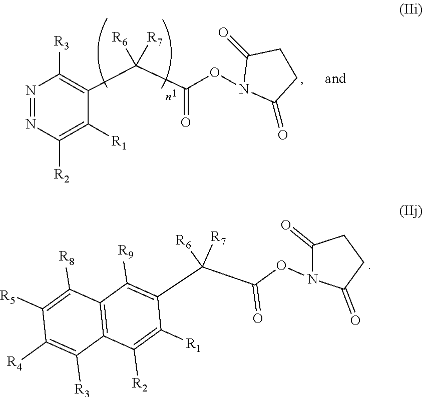

Non-limiting examples of starting materials in which X is a substituted phenyl ring bound to an acetic acid chain include:
a) 2-(difluoromethyl)-4-ethyl-5-methyl-benzeneacetic acid (CAS 2387367-77-1);
b) 4-chloro-5-(difluoromethyl)-2-fluoro-benzeneacetic acid (CAS 2387364-97-6);
c) 4-chloro-2-iodo-6-(trifluoromethyl)-benzeneacetic acid (CAS 2387354-96-1);
d) 5-iodo-2-methyl-3-(trifluoromethyl)-benzeneacetic acid (CAS 2387342-09-6);
e) 3-chloro-6-(difluoromethyl)-2-iodo-benzeneacetic acid (CAS 2387341-72-0);
f) 4-(difluoromethyl)-2-ethyl-6-methyl-benzeneacetic acid (CAS 2387329-36-2);
g) 2-(difluoromethyl)-5-ethyl-4-methyl-benzeneacetic acid (CAS 2387321-04-0);
h) 4-bromo-2-methyl-5-(trifluoromethyl)-benzeneacetic acid (CAS 2387305-32-8);
i) 2-(difluoromethyl)-6-fluoro-3-methyl-benzeneacetic acid (CAS 2387292-94-4);
j) 3-chloro-4-fluoro-5-iodobenzeneacetic acid (CAS 2387289-42-9); k) 3-(difluoromethyl)-5-ethyl-2-methyl-benzeneacetic acid (CAS 2387288-41-5);
l) 3-bromo-5-methyl-2-(trifluoromethyl)-benzeneacetic acid (CAS 2387285-02-9);
m) 2-bromo-4-fluoro-5-(trifluoromethyl)-benzeneacetic acid (CAS 2387265-77-0);
n) 3-(difluoromethyl)-5-iodo-4-methyl-benzeneacetic acid (CAS 2387260-33-3);
o) 3-chloro-2-methyl-6-(trifluoromethyl)-benzeneacetic acid (CAS 2387251-00-3);
p) 4-(difluoromethyl)-3,5-diiodo-benzeneacetic acid (CAS 2387228-43-3);
q) 6-chloro-3-iodo-2-(trifluoromethyl)-benzeneacetic acid (CAS 2387213-04-7);
r) 3-bromo-4-chloro-2-(trifluoromethyl)-benzeneacetic acid (CAS 2387212-65-7);
s) 2-(difluoromethyl)-4-iodo-5-methyl-benzeneacetic acid (CAS 2387172-74-7);
t) 3-(difluoromethyl)-2,6-dimethyl-benzeneacetic acid (CAS 2387166-75-6);
u) 4-fluoro-2-methyl-3-(trifluoromethyl)-benzeneacetic acid (CAS 2387146-20-3);
v) 5-fluoro-4-methyl-2-(trifluoromethyl)-benzeneacetic acid (CAS 2387142-35-8);
w) 2-fluoro-6-iodo-3-(trifluoromethyl)-benzeneacetic acid (CAS 2387142-33-6);
x) 5-(difluoromethyl)-3-ethyl-2-fluoro-benzeneacetic acid (CAS 2387126-96-5);
y) 3-fluoro-5-methyl-4-(trifluoromethyl)-benzeneacetic acid (CAS 2387124-99-2);
z) 3-(difluoromethyl)-4-ethyl-benzeneacetic acid (CAS 2387118-69-4);
aa) 2-(difluoromethyl)-5-fluoro-4-methyl-benzeneacetic acid (CAS 2387117-84-0);
bb) 3-bromo-4-fluoro-2-(trifluoromethyl)-benzeneacetic acid (CAS 2387096-58-2);
cc) 2-(difluoromethyl)-6-ethyl-benzeneacetic acid (CAS 2387059-13-2);
dd) 4-(difluoromethyl)-3-ethyl-2-fluoro-benzeneacetic acid (CAS 2386958-85-4);
ee) 4-(difluoromethyl)-2-fluoro-3-iodo-benzeneacetic acid (CAS 2386902-22-1);
ff) 2-(difluoromethyl)-3-fluoro-6-methyl-benzeneacetic acid (CAS 2386897-89-6);
gg) 3-chloro-2-methyl-4-(trifluoromethyl)-benzeneacetic acid (CAS 2386874-41-3);
hh) 2-(difluoromethyl)-3-fluoro-5-methyl-benzeneacetic acid (CAS 2386844-79-5);
ii) 4-(difluoromethyl)-2-fluoro-6-methyl-benzeneacetic acid (CAS 2386824-64-0);
jj) 3-fluoro-2-methyl-6-(trifluoromethyl)-benzeneacetic acid (CAS 2386725-76-2);
kk) 5-fluoro-4-iodo-2-(trifluoromethyl)-benzeneacetic acid (CAS 2386686-44-6);
ll) 2-bromo-4-fluoro-3-(trifluoromethyl)-benzeneacetic acid (CAS 2386609-80-7);
mm) 3-fluoro-5-iodo-4-(trifluoromethyl)-benzeneacetic acid (CAS 2386573-15-3);
nn) 2-(3,4-difluoro-2-(trifluoromethyl)phenyl)acetic acid (CAS 2386536-41-8);
oo) α-fluoro-2-(trifluoromethyl)-(αR)-benzeneacetic acid (CAS 2382389-60-6);
pp)(R)-2-(2,6-difluorophenyl)-2-fluoroacetic acid (CAS 2382348-82-3);
qq) α,α-difluoro-4-(1,1,2,2,2-pentafluoroethyl)-benzeneacetic acid (CAS 2357382-05-7);
rr) α,α-difluoro-3-(1,1,2,2,2-pentafluoroethyl)-benzeneacetic acid (CAS 2355684-72-7);
ss) α,α-difluoro-3,5-bis(trifluoromethyl)-benzeneacetic acid (CAS 2244941-42-0);
tt) α,α,2,3,6-pentafluoro-benzeneacetic acid (CAS 2228911-75-7);
uu) α,α,2,4,6-pentafluoro-benzeneacetic acid (CAS 2228838-88-6);
vv) α,α,5-trifluoro-2-(trifluoromethyl)-benzeneacetic acid (CAS 2228728-13-8);
ww) α,α,2-trifluoro-4-(trifluoromethyl)-benzeneacetic acid (CAS 2228693-31-8);
xx) α,α,2-trifluoro-3-(trifluoromethyl)-benzeneacetic acid (CAS 2228669-85-8);
yy) α,α,2,3,4,5-hexafluoro-benzeneacetic acid (CAS 2228563-54-8);
zz) α,α,2,3,5,6-hexafluoro-benzeneacetic acid (CAS 2228516-64-9);
aaa) α,α,3-trifluoro-5-(trifluoromethyl)-benzeneacetic acid (CAS 2228224-64-2);
bbb) 4-(difluoromethyl)-α-fluoro-benzeneacetic acid (CAS 2138522-52-6);
ccc) α,α,2-trifluoro-5-(trifluoromethyl)-benzeneacetic acid (CAS 2228136-25-0);
ddd) 3-(difluoromethyl)-α-fluoro-benzeneacetic acid (CAS 2138066-99-4);
eee) 4-(difluoromethyl)-α,α-difluoro-benzeneacetic acid (CAS 2138043-77-1);
fff) 2,5-difluoro-3-(trifluoromethyl)-benzeneacetic acid (CAS 2092866-67-4);
ggg) 3,5-difluoro-2-(trifluoromethyl)-benzeneacetic acid (CAS 2091889-94-8);
hhh) α,α,3-trifluoro-4-(trifluoromethyl)-benzeneacetic acid (CAS 1925367-64-1);
iii) 4-(cyclopropyldifluoromethyl)-benzeneacetic acid (CAS 1896969-02-0);
jjj) 3-(cyclopropyldifluoromethyl)-benzeneacetic acid (CAS 1895738-57-4);
kkk) α,3,4,5-tetrafluoro-benzeneacetic acid (CAS 1880969-10-7);
lll) α,α,3,4,5-pentafluoro-benzeneacetic acid (CAS 1876640-60-6); mmm) 2,4-difluoro-6-(trifluoromethyl)-benzeneacetic acid (CAS 1823551-72-9);
nnn) 2,4-difluoro-3-(trifluoromethyl)-benzeneacetic acid (CAS 1823268-51-4);
ooo) 2-fluoro-4,5-bis(trifluoromethyl)-benzeneacetic acid (CAS 1807026-26-1);
ppp) 3-fluoro-2,5-bis(trifluoromethyl)-benzeneacetic acid (CAS 1807109-79-0);
qqq) 2-fluoro-3,6-bis(trifluoromethyl)-benzeneacetic acid (CAS 1806050-60-1);
rrr) 2,3-bis(trifluoromethyl)-benzeneacetic acid (CAS 1805593-32-1);
sss) 3-fluoro-2,4-bis(trifluoromethyl)-benzeneacetic acid (CAS 1805584-52-4);
ttt) α-fluoro-3-(trifluoromethyl)-benzeneacetic acid (CAS 1517480-45-3);
uuu) 2,5-bis(difluoromethyl)-benzeneacetic acid (CAS 1373827-32-7);
vvv) 2,3,4,6-tetrafluoro-benzeneacetic acid (CAS 1214373-68-8);
www) 2,3,6-trifluoro-4-(trifluoromethyl)-benzeneacetic acid (CAS 1111737-49-5);
xxx) 2,3,4,5-tetrafluoro-6-(trifluoromethyl)-benzeneacetic acid (CAS 1000553-80-9);
yyy) 4-(difluoromethyl)-benzeneacetic acid (CAS);
zzz) 2,6-difluoro-4-(trifluoromethyl)-benzeneacetic acid (CAS 1000517-21-4);
aaaa) α,2-difluoro-benzeneacetic acid (CAS 915070-97-2);
bbbb) 2,3,4,5-tetrafluoro-benzeneacetic acid (CAS 261952-21-0);
cccc) 2,3,4-trifluoro-benzeneacetic acid (CAS 243666-12-8);
dddd) 2,4,6-trifluoro-benzeneacetic acid (CAS 209991-63-9);
eeee) 3,4,5-trifluoro-benzeneacetic acid (CAS 209991-62-8);
ffff) 2,3,4,5,6-pentafluoro-benzene acetic acid (CAS 653-21-4);
gggg) α,α,3,5-tetrafluoro-benzeneacetic acid (CAS);
hhhh) α,3,5-trifluoro-benzeneacetic acid (CAS 208259-38-5);
iiii) 3,5-difluoro-4-(trifluoromethyl)-benzeneacetic acid (CAS 132992-26-8);
jjjj) α,α,4-trifluoro-benzeneacetic acid (CAS 94010-78-3);
kkkk) α,α,2,3,4,5,6-heptafluoro-benzeneacetic acid (CAS 91407-89-5);
llll) 3,5-bis(trifluoromethyl)-benzeneacetic acid (CAS 85068-33-3);
mmmm) α,α-difluoro-4-(trifluoromethyl)-benzeneacetic acid (CAS 73790-11-1);
nnnn) α-fluoro-4-(trifluoromethyl)-benzeneacetic acid (CAS 142044-52-8);
oooo) 4-(trifluoromethyl)-benzeneacetic acid (CAS 32857-62-8);
pppp) 2-(trifluoromethyl)-benzeneacetic acid (CAS 3038-48-0);
qqqq) 2,3,5,6-tetrafluoro-benzeneacetic acid (CAS 3516-91-4);
rrrr) 2-fluoro-benzeneacetic acid (CAS 451-82-1);
ssss) 3-fluoro-benzeneacetic acid (CAS 331-25-9);
tttt) 4-fluoro-benzeneacetic acid (CAS 405-50-5);
uuuu) 2-(4-(pentafluoro-λ⁶-sulfaneyl)phenyl)acetic acid (CAS 1839048-22-4);
vvvv) 2-(2-fluoro-4-(pentafluoro-λ⁶-sulfaneyl)phenyl)acetic acid (CAS 1240257-93-5);
wwww) 2-(3-fluoro-5-(pentafluoro-λ⁶-sulfaneyl)phenyl)acetic acid (CAS 1240257-84-4);
xxxx) 2-(3-(pentafluoro-λ⁶-sulfaneyl)phenyl)acetate (CAS 1211517-00-8);
yyyy) 4-(1-fluoroethyl)-benzeneacetic acid (CAS 1785087-42-4); zzzz) 3-bromo-4-(1-fluoroethyl)-benzeneacetic acid (CAS 1781001-75-9);
aaaaa) 2-chloro-4-(1-fluoroethyl)-benzeneacetic acid (CAS 1783534-87-1);
bbbbb) 4-(1,1,2,2-tetrafluoroethyl)-benzeneacetic acid (CAS 1780654-06-9); and
ccccc) 4-(1,2,2,2-tetrafluoroethyl)-benzeneacetic acid (CAS 1785167-81-8).

Non-limiting examples of starting materials in which X is a substituted phenyl ring bound to a propanoic acid chain include:
a) 3-(3,4,5-trifluorophenyl)propanoic acid (CAS 886499-50-9);
b) 3-(4-(trifluoromethyl)phenyl)propanoic acid (CAS 53473-36-2);
c) (αS)-α,2,5-trifluoro-benzenepropanoic acid (CAS 2382690-89-1);
d) (αS)-α,2,4-trifluoro-benzenepropanoic acid (CAS 2382377-15-1);
e) (αS)-2-chloro-α,3,6-trifluoro-benzenepropanoic acid (CAS 2382372-20-3);
f) (αS)-4-chloro-α,3-difluoro-benzenepropanoic acid (CAS 2382266-52-4);
g) (αS)-α,3,5-trifluoro-benzenepropanoic acid (CAS 2382240-10-8);
h) (αS)-α,2,3,4-tetrafluoro-benzenepropanoic acid (CAS 2382071-54-5);
i) (αS)-α,3,4,5-tetrafluoro-benzenepropanoic acid (CAS 2382062-71-5);
j) (S)-3-(3-chloro-5-fluorophenyl)-2-fluoropropanoic acid (CAS 2381761-11-9);
k) (αS)-3-chloro-α,2-difluoro-benzenepropanoic acid (CAS 2381700-00-9);
l) (αS)-α,2,3-trifluoro-benzenepropanoic acid (CAS 2381635-05-6);
m) (αS)-α,2,4,5-tetrafluoro-benzenepropanoic acid (CAS 2381542-70-5);
n) (αS)-α,2-difluoro-benzenepropanoic acid (CAS 2381458-82-6);
o) (αS)-2-chloro-α,6-difluoro-benzenepropanoic acid (CAS 2381410-60-0);
p) (αS)-3-chloro-α,4-difluoro-benzenepropanoic acid (CAS 2381098-01-5);
q) (αS)-5-chloro-α,2-difluoro-benzenepropanoic acid (CAS 2380960-65-4);
r) (αS)-4-chloro-α,2-difluoro-benzenepropanoic acid (CAS 2380855-42-9);
s) (αS)-α,2,6-trifluoro-benzenepropanoic acid (CAS 2380596-78-9);
t) (αS)-α,3,4-trifluoro-benzenepropanoic acid (CAS 2380495-55-4);
u) 2-bromo-α,α-difluoro-5-(trifluoromethyl)-benzenepropanoic acid (CAS 2360368-80-3);
v) α,α,2-trifluoro-5-iodo-benzenepropanoic acid (CAS 2360274-96-8);
w) 2-bromo-β,β-difluoro-4-(trifluorornethyl)-benzenepropanoic acid (CAS 2360075-86-9);
x) 2-bromo-α,α-difluoro-4-(trifluoromethyl)-benzenepropanoic acid (CAS 2359201-07-1);
y) 3-(4-bromo-2-(trifluoromethyl)phenyl)-2,2-difluoropropanoic acid (CAS 2359191-43-6);
z) β,β-difluoro-4-(1,1,2,2,2-pentafluoroethyl)-benzenepropanoic acid (CAS 2359001-84-4);
aa) α,α-difluoro-3-(1,1,2,2,2-pentafluoroethyl)-benzenepropanoic acid (CAS 2358735-56-3);
bb) 4-bromo-α,α-difluoro-3-(trifluoromethyl)-benzenepropanoic acid (CAS 2358678-40-5);
cc) 3-bromo-β,β-difluoro-5-(trifluoromethyl)-benzenepropanoic acid (CAS 2358114-21-1);
dd) β,β-difluoro-3,5-bis(trifluoromethyl)-benzenepropanoic acid (CAS 2357694-18-7);
ee) 3-bromo-α,α,2,4-tetrafluoro-benzenepropanoic acid (CAS 2357450-02-1);
ff) α,α,4,5-tetrafluoro-2-iodo-benzenepropanoic acid (CAS 2357320-28-4);
gg) 6-bromo-3-chloro-α,α,2-trifluoro-benzenepropanoic acid (CAS 2357291-57-5);
hh) α,α-difluoro-4-(1,1,2,2,2-pentafluoroethyl)-benzenepropanoic acid (CAS 2356957-11-2);
ii) β,β-difluoro-3-iodo-5-(trifluorornethyl)-benzenepropanoic acid (CAS 2356935-06-1);
jj) β,β,2-trifluoro-5-iodo-benzenepropanoic acid (CAS 2356884-34-7);
kk) β,β,4-trifluoro-2-iodo-benzenepropanoic acid (CAS 2356726-93-5);
ll) 2-chloro-β,β-difluoro-4-(trifluoromethyl)-benzenepropanoic acid (CAS 2356702-96-8);
mm) 3-(4-bromo-2-(trifluoromethyl)phenyl)-3,3-difluoropropanoic acid (CAS 2356457-27-5);
nn) 3-bromo-α,α-difluoro-5-(trifluoromethyl)-benzenepropanoic acid (CAS 2356381-90-1);
oo) α,α-difluoro-3,5-bis(trifluoromethyl)-benzenepropanoic acid (CAS 2356328-30-6);
pp) 4-bromo-β,β-difluoro-3-(trifluoromethyl)-benzenepropanoic acid (CAS 2356118-99-3);
qq) 4-bromo-β,β,2,6-tetrafluoro-benzenepropanoic acid (CAS 2355932-06-6);
rr) β,β,4,5-tetrafluoro-2-iodo-benzenepropanoic acid (CAS 2355877-20-0);
ss) 2-bromo-α,α,4,5-tetrafluoro-benzenepropanoic acid (CAS 2355860-44-3);
tt) 2-chloro-α,α-difluoro-4-(trifluoromethyl)-benzenepropanoic acid (CAS 2355830-63-4);
uu) 2-bromo-β,β-difluoro-5-(trifluoromethyl)-benzenepropanoic acid (CAS 2355530-36-6);
vv) α,α,4-trifluoro-2-Iodo-benzenepropanoic acid (CAS 2354996-90-8); ww) 2-bromo-β,β,4,5-tetrafluoro-benzenepropanoic acid (CAS 2354951-14-5);
xx) 3-bromo-β,β,2,4-tetrafluoro-benzenepropanoic acid (CAS 2354947-62-7);
yy) 6-bromo-3-chloro-β,β,2-trifluoro-benzenepropanoic acid (CAS 2354922-42-0);
zz) β,β-difluoro-3-(1,1,2,2,2-pentafluoroethyl)-benzenepropanoic acid (CAS 2354790-33-1);
aaa) α,α-difluoro-3-iodo-5-(trifluoromethyl)-benzenepropanoic acid (CAS 2354788-36-4);
bbb) 4-(1,1,2,2,2-pentafluoroethyl)-benzenepropanoic acid (CAS 2354177-10-7);
ccc) 3-(1,1,2,2,2-pentafluoroethyl)-benzenepropanoic acid (CAS 2354163-50-9);
ddd) 4,5-difluoro-2-iodo-benzenepropanoic acid (CAS 2352673-92-6);
eee) 3-fluoro-benzenepropanoic acid (CAS 2300968-80-1 as Na salt);
fff) β,β,2-trifluoro-3-(trifluoromethyl)-benzenepropanoic acid (CAS 2229622-24-4);
ggg) 3-bromo-α,α,4-trifluoro-benzenepropanoic acid (CAS 2229605-71-2);
hhh) 3-bromo-β,β,2-trifluoro-benzenepropanoic acid (CAS 2229592-38-3);
iii) 3-chloro-β,β,2-trifluoro-benzenepropanoic acid (CAS 2229590-96-7);
jjj) β,β,3,5-tetrafluoro-benzenepropanoic acid (CAS 2229567-47-7);
kkk) 2-chloro-β,β,4,5-tetrafluoro-benzenepropanoic acid (CAS 2229558-44-3);
lll) 4-chloro-β,β,2-trifluoro-benzenepropanoic acid (CAS 2229546-10-3);
mmm) α,α,4-trifluoro-3-(trifluoromethyl)-benzenepropanoic acid (CAS 2229533-27-9);
nnn) 3-chloro-β,β,5-trifluoro-benzenepropanoic acid (CAS 2229531-21-7);
ooo) 2-chloro-α,α,4-trifluoro-benzenepropanoic acid (CAS 2229519-83-7);
ppp) 2,4-dichloro-5-fluoro-benzenepropanoic acid (CAS 2229501-39-5);
qqq) 4-chloro-α,α,2,5-tetrafluoro-benzenepropanoic acid (CAS 2229495-59-2);
rrr) α,α,4-trifluoro-2-(trifluoromethyl)-benzenepropanoic acid (CAS 2229485-71-4);
sss) β,β,2,3,5,6-hexafluoro-benzenepropanoic acid (CAS 2229477-47-6);
ttt) 4-bromo-β,β,3-trifluoro-benzenepropanoic acid (CAS 2229456-75-9);
uuu) 3-bromo-β,β,4-trifluoro-benzenepropanoic acid (CAS 2229439-59-0);
vvv) 3-chloro-α,α,2-trifluoro-benzenepropanoic acid (CAS 2229421-53-6);
www) β,β,2,3,6-pentafluoro-benzenepropanoic acid (CAS 2229417-51-8);
xxx) β,β,3-trifluoro-5-(trifluoromethyl)-benzenepropanoic acid (CAS 2229410-48-2);
yyy) β,β,4-trifluoro-2-(trifluoromethyl)-benzenepropanoic acid (CAS 2229394-98-1);
zzz) 2-chloro-β,β,3,6-tetrafluoro-benzenepropanoic acid (CAS 2229265-32-9);
aaaa) 2-chloro-α,α-difluoro-6-(trifluoromethyl)-benzenepropanoic acid (CAS 2229240-08-6);
bbbb) 3-chloro-α,α,2,6-tetrafluoro-benzenepropanoic acid (CAS 2229218-62-4);
cccc) 2-chloro-α,α,3-trifluoro-benzenepropanoic acid (CAS 2229205-15-4);
dddd) α,α,2,3,4,5,6-heptafluoro-benzenepropanoic acid (CAS 2229177-33-5);
eeee) 4-chloro-α,α-difluoro-2-(trifluoromethyl)-benzenepropanoic acid (CAS 2229175-23-7);
ffff) β,β2,4,5-pentafluoro-benzenepropanoic acid (CAS 2229101-31-7); gggg) 2-chloro-α,α,4,5-tetrafluoro-benzenepropanoic acid (CAS 2229008-37-9);
hhhh) 3-bromo-β,β,5-trifluoro-benzenepropanoic acid (CAS 2228975-31-1);
iiii) 2-chloro-α,α-difluoro-5-(trifluorornethyl)-benzenepropanoic acid (CAS 2228970-45-2);
jjjj) α,α,2,4,6-pentafluoro-benzenepropanoic acid (CAS 2228950-57-8);
kkkk) α,α,2,3,4,5-hexafluoro-benzenepropanoic acid (CAS 2228912-69-2);
llll) β,β,5-trifluoro-2-(trifluoromethyl)-benzenepropanoic acid (CAS 2228902-90-5);
mmmm) 4-chloro-α,α-difluoro-3-(trifluoromethyl)-benzenepropanoic acid (CAS 2228900-61-4);
nnnn) 2,6-bis(trifluoromethyl)-benzenepropanoic acid (CAS 1806540-97-5);
oooo) α,α,2-trifluoro-3-(trifluoromethyl)-benzenepropanoic acid (CAS 2228840-45-5);
pppp) 2-chloro-β,β,3-trifluoro-benzenepropanoic acid (CAS 2228838-55-7);
qqqq) β,β,2-trifluoro-5-(trifluoromethyl)-benzenepropanoic acid (CAS 2228834-61-3);
rrrr) 4-bromo-β,β,2-trifluoro-benzenepropanoic acid (CAS 2228810-79-3);
ssss) β,β,2,3,4,5,6-heptafluoro-benzenepropanoic acid (CAS 2228770-72-5);
tttt)β,β,2,3,4-pentafluoro-benzenepropanoic acid (CAS 2228761-28-0); uuuu) α,α,2,3,6-pentafluoro-benzenepropanoic acid (CAS 2228740-39-2);
vvvv) α,α,2-trifluoro-4-(trifluoromethyl)-benzenepropanoic acid (CAS 2228301-97-9);
wwww) β,β,2-trifluoro-4-(trifluorornethyl)-benzenepropanoic acid (CAS 2228306-35-0);
xxxx) α,α,3-trifluoro-4-(trifluoromethyl)-benzenepropanoic acid (CAS 2228226-24-0);
yyyy) 2-(trifluorornethyl)-benzenepropanoic acid (CAS 94022-99-8); zzzz) 3,4-bis(trifluoromethyl)-benzenepropanoic acid (CAS 1421281-58-4);
aaaaa) 2,4-bis(trifluoromethyl)-benzenepropanoic acid (CAS 1092460-63-3);
bbbbb) 2,5-bis(trifluorornethyl)-benzenepropanoic acid (CAS 302912-03-4);
ccccc) 2-(difluoromethyl)-6-(trifluoromethyl)-benzenepropanoic acid (CAS 1261606-03-4);
ddddd) 3-(difluoromethyl)-2-(trifluoromethyl)-benzenepropanoic acid (CAS 1261878-33-4);
eeeee) 4-(difluoromethyl)-2-(trifluoromethyl)-benzenepropanoic acid (CAS 1261677-41-1);
fffff) 5-(difluoromethyl)-2-(trifluoromethyl)-benzenepropanoic acid (CAS 1261617-95-1);
ggggg) 2-(difluoromethyl)-6-(trifluoromethyl)-benzenepropanoic acid (CAS 1261606-03-4);
hhhhh) 4-chloro-β,β-difluoro-3-(trifluoromethyl)-benzenepropanoic acid (CAS 2228731-08-4);
iiiii) 3-chloro-β,β-difluoro-4-(trifluoromethyl)-benzenepropanoic acid (CAS 2228729-67-5);
jjjjj) (S)-4-(1-fluoroethyl)-benzenepropanoic acid (CAS 162327-95-9);
kkkkk) 4-(1-fluoroethyl)-benzenepropanoic acid (CAS 1780941-12-9);
lllll) 3-bromo-4-(1-fluoroethyl)-benzenepropanoic acid (CAS 1784269-24-4);
mmmmm) 2-chloro-4-(1-fluoroethyl)-benzenepropanoic acid (CAS 1782851-21-1);
nnnnn) 4-(1,1,2,2-tetrafluoroethyl)-benzenepropanoic acid (CAS 1781489-51-7); and
ooooo) 4-(1,2,2,2-tetrafluoroethyl)-benzenepropanoic acid (CAS 1785140-35-3).

Non-limiting examples of starting materials in which X is a substituted phenyl ring bound to a butanoic acid chain include:
a) 4-(2,4,5-trifluorophenyl)butanoic acid (CAS 1258638-46-8);
b) 4-(4-bromo-2,3-difluorophenyl)butanoic acid (CAS 1891704-35-0);
c) 4-(3-bromo-2,4-difluorophenyl)butanoic acid (CAS 1898360-11-6);
d) 4-(3,5-difluoro-2-methylphenyl)butanoic acid (CAS 1895515-05-5);
e) 4-(4-bromo-2,5-difluorophenyl)butanoic acid (CAS 1343072-28-5);
f) 4-(2,3,5-trifluorophenyl)butanoic acid (CAS 1892694-42-6);
g) 4-(2,3,6-trifluorophenyl)butanoic acid (CAS 1895584-02-7);
h) 4-(2,4-difluoro-3-methylphenyl)butanoic acid (CAS 1895503-43-1);
i) 4-(2,4,6-trifluorophenyl)butanoic acid (CAS 1042558-67-7);
j) 4-(2,3,4,5-tetrafluorophenyl)butanoic acid (CAS 1866658-81-2);
k) 4-(2,3-difluoro-5-isopropylphenyl)butanoic acid (CAS 1891501-56-6);
l) 4-(2,6-difluoro-4-methylphenyl)butanoic acid (CAS 2228602-62-6);
m) 4-(2,3,5,6-tetrafluorophenyl)butanoic acid (CAS 1852009-46-1);
n) 4-(4-chloro-2,6-difluorophenyl)butanoic acid (CAS 1891481-94-9);
o) 4-(4-bromo-2,6-difluorophenyl)butanoic acid (CAS 1891821-67-2);
p) 4-(3,4-difluoro-5-methylphenyl)butanoic acid (CAS 1891439-79-4);
q) 4-(perfluorophenyl)butanoic acid (CAS 1892073-55-0);
r) 4-(3,5-difluoro-4-methylphenyl)butanoic acid (CAS 1895437-98-5);
s) 4-(4-chloro-2,3,5,6-tetrafluorophenyl)butanoic acid (CAS 1892694-54-0);
t) 4-(4-bromo-2,3,5,6-tetrafluorophenyl)butanoic acid (CAS 1892860-77-3);
u) 4-(2,5-difluoro-4-methylphenyl)butanoic acid (CAS 1515548-81-8);
v) 4-(2-(trifluoromethyl)phenyl)butanoic acid (CAS 899350-21-1);
w) 4-(4-(1,1-difluoroethyl)phenyl)butanoic acid (CAS 1892107-54-8);
x) 4-(4-(2,2-difluoroethyl)phenyl)butanoic acid (CAS 1891678-12-8);
y) 4-(4-(1,1-difluoropropyl)phenyl)butanoic acid (CAS 1893756-51-8);
z) 4-(4-(2,2,2-trifluoroethyl)phenyl)butanoic acid (CAS 1898423-39-6);
aa) 4-(6-(trifluoromethyl)pyridin-3-yl)butanoic acid (CAS 1100766-80-0);
bb) 4-(5-(trifluoromethyl)pyridin-2-yl)butanoic acid (CAS 1100766-65-1);
cc) 4-(3,4,5-trifluorophenyl)butanoic acid (CAS 1410187-01-7);
dd) 4-(2,3,4-trifluorophenyl)butanoic acid (CAS 1368465-86-4);
ee) 4-(3-(fluoromethyl)phenyl)butanoic acid (CAS 1895587-73-1);
ff) 4-(4-(fluoromethyl)phenyl)butanoic acid (CAS 1896663-99-2);
gg) 4-(4-(difluoromethyl)phenyl)butanoic acid (CAS 1549717-55-6);
hh) 4-(3-(1,1-difluoroethyl)phenyl)butanoic acid (CAS 1897047-82-3);
ii) 4-(2-(1,1-difluoroethyl)phenyl)butanoic acid (CAS 1898215-71-8);
jj) 4-(2-(fluoromethyl)phenyl)butanoic acid (CAS 1891284-26-6);
kk) 4-(5-(1,1-difluoroethyl)-2-fluorophenyl)butanoic acid (CAS 1891846-74-7);
ll) 4-(2-(difluoromethyl)phenyl)butanoic acid (CAS 1891439-80-7);
mm) 4-(3-(difluoromethyl)phenyl)butanoic acid (CAS 1550251-61-0);
nn) 4-(2-(difluoromethyl)-3-fluorophenyl)butanoic acid (CAS 1891478-59-3);
oo) 4-(5-(difluoromethyl)-2,3-difluorophenyl)butanoic acid (CAS 1892029-96-7);
pp) 4-(5-(difluoromethyl)-2-fluorophenyl)butanoic acid (CAS 1898306-94-9);
qq) 4-(4-(difluoromethyl)-3-fluorophenyl)butanoic acid (CAS 1897322-37-0);
rr) 4-(3-(trifluoromethyl)phenyl)butanoic acid (CAS 145485-43-4);
ss) 4-(2-fluoro-4-(trifluoromethyl)phenyl)butanoic acid (CAS 1892030-00-0);
tt) 4-(3-fluoro-4-(trifluoromethyl)phenyl)butanoic acid (CAS 2353584-26-4);
uu) 4-(2-fluoro-3-(trifluoromethyl)phenyl)butanoic acid (CAS 1898256-64-8);
vv) 4-(3,5-bis(trifluoromethyl)phenyl)butanoic acid (CAS 184970-19-2);
ww) 4-(3-fluoro-5-(trifluoromethyl)phenyl)butanoic acid (CAS 1558540-24-1);
xx) 4-(4-fluoro-3-(trifluoromethyl)phenyl)butanoic acid (CAS 1552828-44-0);
yy) 4-(2-fluoro-5-(trifluoromethyl)phenyl)butanoic acid (CAS 1538963-44-8);
zz) 4-(3-fluoro-2-(trifluoromethyl)phenyl)butanoic acid (CAS 1892036-09-7);
aaa) 4-(5-fluoro-2-(trifluoromethyl)phenyl)butanoic acid (CAS 2353998-39-5);
bbb) 4-(2-fluoro-6-(trifluoromethyl)phenyl)butanoic acid (CAS 1520160-59-1);
ccc) 4-(4-fluoro-2-(trifluoromethyl)phenyl)butanoic acid (CAS 1518823-88-5);
ddd) 4-(4-(1,1-difluoro-2-methylpropyl)phenyl)butanoic acid (CAS 1896965-12-0);
eee) 4-(3-(1,1-difluoro-2-methylpropyl)phenyl)butanoic acid (CAS 1895736-10-3);
fff) 4-(3-(1,1-difluorobutyl)phenyl)butanoic acid (CAS 1894439-30-5);
ggg) 4-(4-(cyclopropyldifluoromethyl)phenyl)butanoic acid (CAS 1893758-75-2);
hhh) 4-(3-(1,1-difluoropropyl)phenyl)butanoic acid (CAS 1893752-08-3);
iii) 4-(4-(1,1-difluorobutyl)phenyl)butanoic acid (CAS 1892501-36-8);
jjj) 4-(4-(2,2-difluoropropyl)phenyl)butanoic acid (CAS);
kkk) 4-(3-(2,2-difluoropropyl)phenyl)butanoic acid (CAS 1898074-87-4);
lll) (CAS);
mmm) 4-(3-(cyclopropyldifluoromethyl)phenyl)butanoic acid (CAS 1892502-75-8);
nnn) 4-(3-(2-fluoroethyl)phenyl)butanoic acid (CAS 1895492-10-0);
ooo) 4-(4-(perfluoroethyl)phenyl)butanoic acid (CAS 235997-34-1);
ppp) 4-(3-(perfluoroethyl)phenyl)butanoic acid (CAS 2359485-05-3);
qqq) 4-(4-(2,2,2-trifluoroethyl)phenyl)butanoic acid (CAS 1897395-89-9);
rrr) 4-(3-(2,2-difluoroethyl)phenyl)butanoic acid (CAS 1897137-40-4);
sss) 4-(2-(2,2-difluoroethyl)phenyl)butanoic acid (CAS 1898129-58-2);
ttt) 4-(2-(2-fluoropropyl)phenyl)butanoic acid (CAS 1897883-67-8);
uuu) 4-(2-(2,2-difluoropropyl)phenyl)butanoic acid (CAS 1891501-64-6);
vvv) 4-(2-(2-fluoro-2-methylpropyl)phenyl)butanoic acid (CAS 1897177-32-0);
www) 4-(3-(1,1-difluoro-2-methylpropyl)phenyl)butanoic acid (CAS 1895736-10-3);
xxx) 4-(3-(2-fluoropropyl)phenyl)butanoic acid (CAS 1891542-35-0);
yyy) 4-(4-(2-fluoroethyl)phenyl)butanoic acid (CAS);
zzz) 4-(4-(3-fluoropropyl)phenyl)butanoic acid (CAS 1898181-97-9);
aaaa) 4-(4-(3,3-difluoropropyl)phenyl)butanoic acid (CAS 1898047-82-9);
bbbb) 4-(4-(1,1,1,3,3,3-hexafluoropropan-2-yl)phenyl)butanoic acid (CAS 1898342-32-9);
cccc) 2-chloro-4-(1-fluoroethyl)-benzenebutanoic acid (CAS 1898404-80-2);
dddd) 3-bromo-4-(1-fluoroethyl)-benzenebutanoic acid (CAS 1892251-33-0)
eeee) 4-(1-fluoroethyl)-benzenebutanoic acid (CAS 1895445-18-7);
ffff) 5-(1,1,-difluoroethyl)-2-fluoro-benzenebutanoic acid (CAS 1891846-74-4); and
gggg) 4-(4-(perfluoropropyl)phenyl)butanoic acid (CAS 1802226-54-5).

Non-limiting examples of starting materials in which X is a substituted phenyl ring bound to a pentanoic acid chain include:
a) 5-(2,4-difluorophenyl)pentanoic acid (CAS 1258638-46-8);
b) 5-(2,4,5-trifluorophenyl)pentanoic acid (CAS 1258638-06-0);
c) β,β,4-trifluoro-benzenepentanoic acid (CAS 2357850-48-5);
d) 2,3,4-trifluoro-benzenepentanoic acid (CAS 2144094-39-1);
e) 2,4,6-trifluoro-benzenepentanoic acid (CAS 1039855-30-5);
f) 3-bromo-2,4-difluoro-benzenepentanoic acid (CAS 2411288-48-5);
g) 2-chloro-3,6-difluoro-benzenepentanoic acid (CAS 2160835-66-3);
h) 4-chloro-2-fluoro-benzenepentanoic acid (CAS 2029859-36-5);
i) 2-bromo-6-fluoro-benzenepentanoic acid (CAS 2029732-19-0);
j) 3-bromo-5-fluoro-benzenepentanoic acid (CAS 2025200-26-2);
k) 4-chloro-3-fluoro-benzenepentanoic acid (CAS 2024866-57-5);
l) 2-bromo-4-fluoro-benzenepentanoic acid (CAS 2023507-80-2);
m) 2-chloro-6-fluoro-benzenepentanoic acid (CAS 2023189-02-6);
n) 3-chloro-2-fluoro-benzenepentanoic acid (CAS 2008328-45-6);
o) 6-bromo-2,3,4-trifluoro-benzenepentanoic acid (CAS 2007899-85-4);
p) 4-bromo-2-fluoro-benzenepentanoic acid (CAS 2006819-16-3);
q) 2-bromo-5-fluoro-benzenepentanoic acid (CAS 2006572-85-4);
r) 3-bromo-2-fluoro-benzenepentanoic acid (CAS 2005192-30-1);
s) 2-bromo-3-fluoro-benzenepentanoic acid (CAS 2002079-21-0);
t) 3-chloro-2,4-difluoro-benzenepentanoic acid (CAS 2001965-75-7);
u) 3-chloro-2-fluoro-benzenepentanoic acid (CAS 2008328-45-6);
v) 6-bromo-2,3,4-trifluoro-benzenepentanoic acid (CAS 2007899-85-4);
w) 4-bromo-2-fluoro-benzenepentanoic acid (CAS 2006819-16-3);
x) 3-chloro-2,4-difluoro-benzenepentanoic acid (CAS 2001965-75-7);
y) 2-(trifluoromethyl)-benzenepentanoic acid (CAS 1996894-39-3);
z) 3-(trifluoromethyl)-benzenepentanoic acid (CAS 1893536-03-2);
aa) 2,3,5,6-tetrafluoro-benzenepentanoic acid (CAS 1994599-71-1);
bb) 2,3,4,5-tetrafluoro-benzenepentanoic acid (CAS 1994555-37-1);
cc) 3-fluoro-5-(trifluoromethyl)-benzenepentanoic acid (CAS 1989842-02-5);
dd) 4-fluoro-2-(trifluoromethyl)-benzenepentanoic acid (CAS 1984096-41-4);
ee) 4-bromo-5-chloro-5,5,2-trifluoro-benzenepentanoic acid (CAS 1981499-72-2);
ff) 5,5,3,4-tetrafluoro-benzenepentanoic acid (CAS 1039856-91-1);
gg) 5,5,2,4-tetrafluoro-benzenepentanoic acid (CAS 1039856-69-3);
hh) 5,5,2,5-tetrafluoro-benzenepentanoic acid (CAS 1039330-44-3);
ii) 5,5,2,4,5-pentafluoro-benzenepentanoic acid (CAS 1977193-72-8);
jj) 5,6,2,4,6-pentafluoro-benzenepentanoic acid (CAS 1039330-93-2);
kk) 2-chloro-δ,δ,4,5-tetrafluoro-benzenepentanoic acid (CAS 1929988-13-5);
ll) 3,5-difluoro-benzenepentanoic acid (CAS 1700328-22-8);
mm) 2,4-difluoro-benzenepentanoic acid (CAS 1039879-09-8);
nn) 3-(difluoromethyl)-benzenepentanoic acid (CAS 1691674-64-2);
oo) 4-(difluoromethyl)-benzenepentanoic acid (CAS 1698364-72-5);
pp) 2,6-difluoro-benzenepentanoic acid (CAS 1696909-67-7);
qq) 2,3-difluoro-benzenepentanoic acid (CAS 1696342-68-3);
rr) 3,4-difluoro-benzenepentanoic acid (CAS 1037156-75-4);
ss) 2,5-difluoro-benzenepentanoic acid (CAS 944950-25-8);
tt) 3,4,5-trifluoro-benzenepentanoic acid (CAS 1695388-51-2);
uu) 2,4,5-trifluoro-benzenepentanoic acid (CAS 1258638-06-0);
vv) 2-fluoro-benzenepentanoic acid (CAS 1536031-77-2);
ww) 3-fluoro-benzenepentanoic acid (CAS 1057601-93-0);
xx) 4-fluoro-benzenepentanoic acid (CAS 24484-22-8);
yy) α,α,4-trifluoro-benzenepentanoic acid (CAS 1356339-18-8);
zz) δ,δ,4-trifluoro-benzenepentanoic acid (CAS 1038713-64-2); and
aaa) 4-bromo-2,5-difluoro-benzenepentanoic acid (CAS 1339229-25-2).

Non-limiting examples of starting materials in which X is a substituted phenyl ring bound to a hexanoic acid chain include:
a) 6-(2,4,6-trifluorophenyl)hexanoic acid (CAS 1153515-36-6);
b) 6,6-difluoro-6-(2,4,6-trifluorophenyl)hexanoic acid (CAS 1153517-46-4);
c) 6-(2,4,5-trifluorophenyl)hexanoic acid (CAS 1258639-02-9);
d) ε,ε,2,4,5-pentafluoro-benzenehexanoic acid (CAS 19790006-75-1);
e) ε,ε,2,4-tetrafluoro-benzenehexanoic acid (CAS 1156762-08-1);
f) ε,ε,3,4-tetrafluoro-benzenehexanoic acid (CAS 1153517-35-1);
g) ε,ε,4-trifluoro-benzenehexanoic acid (CAS 1153517-16-8);
h) 6-(2,5-difluorophenyl)-6,6-difluorohexanoic acid (CAS 1153517-04-4);
i) 6-(2,4-difluorophenyl)hexanoic acid (CAS 1153515-11-7);
j) 6-(2,5-difluorophenyl)hexanoic acid (CAS 1153515-04-8);
k) 6-(3-fluorophenyl)hexanoic acid (CAS 1057602-73-9);
l) 6-(2,4,5-trifluorophenyl)hexanoic acid (CAS 12158639-02-9);
m) 6-(2-fluorophenyl)hexanoic acid (CAS 1225502-16-8);
n) 4-(trifluoromethyl)-benzenehexanoic acid (CAS 2169947-24-2);
o) 2,3,5,6-tetrafluoro-benzenehexanoic acid (CAS 2064073-04-5);
p) 3,4-difluoro-benzenehexanoic acid (CAS 1156768-39-6);
q) 4-fluoro-benzenehexanoic acid (CAS 89326-72-7);
r) 3-(trifluoromethyl)-benzenehexanoic acid (CAS 79023-02-2);
s) ε,ε-difluoro-3-(trifluoromethyl)-benzenehexanoic acid (CAS 2170123-91-6);
t) 3-chloro-2,4-difluoro-benzenehexanoic acid (CAS 2020718-25-4);
u) 2-chloro-ε,ε,4,5-tetrafluoro-benzenehexanoic acid (CAS 1989914-46-6);
v) 4-bromo-5-chloro-ε,ε,2-trifluoro-benzenehexanoic acid (CAS 1981357-46-3);
w) 4-bromo-5-chloro-2-fluoro-benzenehexanoic acid (CAS 19871357-10-1);
x) 2-chloro-4,5-difluoro-benzenehexanoic acid (CAS 1962264-44-3);
y) 5-chloro-2-fluoro-benzenehexanoic acid (CAS 1906779-22-3);
z) 3-chloro-4-fluoro-benzenehexanoic acid (CAS 1907932-50-6);
aa) ε,4-difluoro-benzenehexanoic acid (CAS 1823137-23-0);
bb) 4-bromo-3-fluoro-benzenehexanoic acid (CAS 1531588-20-1);
cc) 3-bromo-4-fluoro-benzenehexanoic acid (CAS 1516951-41-9);
dd) 3-bromo-ε,ε,4-trifluoro-benzenehexanoic acid (CAS 1508153-39-6);
ee) 4-bromo-ε,ε,2,5-tetrafluoro-benzenehexanoic acid (CAS 1409276-16-9);
ff) 4-bromo-ε,ε,2,5-tetrafluoro-benzenehexanoic acid (CAS 1409276-16-9);
gg) 4-bromo-2,5-difluoro-benzenehexanoic acid (CAS 1408847-17-5);
hh) 2-chloro-6-fluoro-benzenehexanoic acid (CAS 1225733-49-2); and
ii) 2,5-difluoro-benzenehexanoic acid (CAS 1153515-04-8).

Non-limiting examples of starting materials in which X is a substituted phenyl ring bound to a heptanoic acid chain include:
a) 2,3,5,6-tetrafluoro-benzeneheptanoic acid (CAS 2064073-07-8);
b) 2,4,5-trifluoro-benzeneheptanoic acid (CAS 1258639-12-1);
c) 2,4-difluoro-benzeneheptanoic acid (CAS 1258638-05-9);
d) 4-(trifluoromethyl)-benzeneheptanoic acid (CAS 952068-28-9); and
e) 4-fluoro-benzeneheptanoic acid (CAS 952068-26-7).

Non-limiting examples of starting materials in which X is a substituted 2-pyridinyl ring include:
a) 3-bromo-5-fluoro-2-pyridinebutanoic acid (CAS 2385313-06-2);
b) α,α,6-trifluoro-2-pyridinepropanoic acid (CAS 2360067-10-1);
c) 3-(trifluoromethyl)-2-pyridinebutanoic acid (CAS 2360067-10-1);
d) 3-(trifluoromethyl)-2-pyridinebutanoic acid (CAS 2359525-21-4);
e) β,β,6-trifluoro-2-pyridinepropanoic acid (CAS 2358658-27-0);
f) 6-fluoro-2-pyridinebutanoic acid (CAS 2358175-76-3);
g) 5-bromo-3-fluoro-2-pyridinebutanoic acid (CAS 2358090-86-3);
h) 5-bromo-β,β,3-trifluoro-2-pyridinepropanoic acid (CAS 2356556-99-3);
i) 5-bromo-α,α,3-trifluoro-2-pyridinepropanoic acid (CAS 2354910-41-9);
j) 5-(difluoromethyl)-2-pyridinepropanoic acid (CAS 2303431-79-8);
k) 5-fluoro-2-pyridinepentanoic acid (CAS 2273487-21-9);
l) 5-fluoro-2-pyridinepropanoic acid, (CAS 2248336-33-4);
m) β,β,5-trifluoro-2-pyridinepropanoic acid, (CAS 2229635-34-9);
n) 3-fluoro-2-pyridinebutanoic acid (CAS 2229605-83-6);
o) β,β-difluoro-3-(trifluoromethyl)-2-pyridinepropanoic acid, (CAS 2229397-33-3);
p) α,α,5-trifluoro-2-pyridinepropanoic acid (CAS 2229233-71-8);
q) β,β-difluoro-5-(trifluoromethyl)-2-pyridinepropanoic acid, (CAS 2228930-65-0);
r) β,β,3-trifluoro-2-pyridinepropanoic acid (CAS 2228872-67-9);
s) α,α,3-trifluoro-2-pyridinepropanoic acid (CAS 2228831-51-2);
t) α,α-difluoro-3-2-pyridinepropanoic acid (CAS 2228812-00-6);
u) β,β,4-trifluoro-2-pyridinepropanoic acid (CAS 2228760-72-1);
v) α,α-difluoro-5-(trifluoromethyl)-2-pyridinepropanoic acid (CAS 2228522-65-2);
w) α,α,4-trifluoro-2-pyridinepropanoic acid (CAS 2228425-13-4);
x) α,5-difluoro-2-pyridinepropanoic acid (CAS 2142211-84-3);
y) 6-fluoro-2-pyridinepropanoic acid (CAS 1934919-89-7);
z) 3-(trifluoromethyl)-2-pyridinepropanoic acid (CAS 1897547-47-5);
aa) 4-fluoro-2-pyridinepropanoic acid (CAS 1823931-38-9);
bb) 3-(difluoromethyl)-2-pyridinepropanoic acid (CAS 1785088-10-9);
cc) 4-(difluoromethyl)-2-pyridinepropanoic acid (CAS 1783679-85-5);
dd) 5-fluoro-2-pyridinepropanoic acid (CAS 1783569-44-7); and
ee) 6-(difluoromethyl)-2-pyridinepropanoic acid (CAS 1783372-30-4).

Non-limiting examples of starting materials in which X is a substituted 3-pyridinyl ring include:
a) 2-chloro-5-3-pyridinebutanoic acid (CAS 2360168-32-5);
b) 4-chloro-2-fluoro-3-pyridinebutanoic acid (CAS 2359188-55-7);
c) 6-chloro-2-3-pyridinebutanoic acid (CAS 2358933-74-9);
d) 2,6-dichloro-5-fluoro-3-pyridinebutanoic acid (CAS 2358239-44-6);
e) 2-chloro-5-fluoro-3-pyridinebutanoic acid (CAS 2358222-55-4);
f) 4-(trifluoromethyl)-3-pyridinebutanoic acid (CAS 2358080-05-2);
g) 2-(trifluoromethyl)-3-pyridinebutanoic acid (CAS 2357391-49-0);
h) 6-chloro-α,α-difluoro-2-(trifluoromethyl)-3-pyridinepropanoic acid (CAS 2356427-68-2);
i) 6-chloro-β,β-difluoro-2-(trifluoromethyl)-3-pyridinepropanoic acid (CAS 2355146-27-7);
j) 4-chloro-2-fluoro-3-pyridinepropanoic acid (CAS 2355123-00-9);
k) 6-chloro-2-(trifluoromethyl)-3-pyridinepropanoic acid (CAS 2354044-83-8);
l) 2-chloro-5-fluoro-3-pyridinepropanoic acid (CAS 2353114-96-0);
m) 4-chloro-α,α,2-3-pyridinepropanoic acid (CAS 2229566-01-0);
n) β,β-difluoro-2-(trifluoromethyl)-3-pyridinepropanoic acid (CAS 2229452-64-4);
o) 4-chloro-β,β,2-trifluoro-3-pyridinepropanoic acid (CAS 2229401-19-6);
p) 5-bromo-2-fluoro-3-pyridinepropanoic acid (CAS 2229368-01-6);
q) α,α-difluoro-2-(trifluoromethyl)-3-pyridinepropanoic acid (CAS 2229345-18-8);
r) 2,6-dichloro-5-fluoro-3-pyridinepropanoic acid (CAS 2229326-16-1);
s) α,α-difluoro-4-3-pyridinepropanoic acid (CAS 2229303-01-7);
t) α,α,2-trifluoro-3-pyridinepropanoic acid (CAS 2229268-68-0);
u) 5-bromo-2-fluoro-3-pyridinebutanoic acid (CAS 2229227-20-5);
v) α,α-difluoro-6-(trifluoromethyl)-3-pyridinepropanoic acid (CAS 2229172-52-3);
w) 6-fluoro-3-pyridinebutanoic acid (CAS 2229160-24-9);
x) 2-chloro-α,α,5-trifluoro-3-pyridinepropanoic acid (CAS 2229092-66-2);
y) 2-chloro-β,β,5-trifluoro-3-pyridinepropanoic acid (CAS 2228999-30-0);
z) 2-chloro-β,β-difluoro-5-(trifluoromethyl)-3-pyridinepropanoic acid (CAS 2228828-92-8);
aa) 2-chloro-α,α-difluoro-5-(trifluoromethyl)-3-pyridinepropanoic acid (CAS 2228828-83-7);
bb) 2-chloro-5-(trifluoromethyl)-3-pyridinepropanoic acid (CAS 2228811-25-2);
cc) 5-chloro-β,β,2-trifluoro-3-pyridinepropanoic acid (CAS 2228808-58-8);
dd) 2,6-dichloro-α,α,5-trifluoro-3-pyridinepropanoic acid (CAS 2228806-06-0);
ee) β,β-difluoro-4-(trifluoromethyl)-3-pyridinepropanoic acid (CAS 2228790-91-6);
ff) β,β,5-trifluoro-3-pyridinepropanoic acid (CAS 2228687-01-0);
gg) 2-fluoro-3-pyridinebutanoic acid (CAS 2228678-28-0);
hh) 2,6-dichloro-β,β,5-trifluoro-3-pyridinepropanoic acid (CAS 2228593-37-9);
ii) β,β,6-trifluoro-3-pyridinepropanoic acid (CAS 2228592-54-7);
jj) β,β,2-trifluoro-3-pyridinepropanoic acid (CAS 2228582-84-9);
kk) α,α,6-trifluoro-3-pyridinepropanoic acid (CAS 2228582-17-8);
ll) β,β-difluoro-6-(trifluoromethyl)-3-pyridinepropanoic acid (CAS 2228535-87-1);
mm) 5-chloro-α,α,2-trifluoro-3-pyridinepropanoic acid (CAS 2228534-97-0);
nn) 5-chloro-2-fluoro-3-pyridinepropanoic acid (CAS 2228520-72-5);
oo) 5-bromo-α,α,2-trifluoro-3-pyridinepropanoic acid (CAS 2228480-20-2);
pp) 5-bromo-β,β,2-trifluoro-3-pyridinepropanoic acid (CAS 2228475-66-7);
qq) 5-chloro-2-fluoro-3-pyridinebutanoic acid (CAS 2228402-79-5);
rr) 2-(trifluoromethyl)-3-pyridinepropanoic acid (CAS 2142222-23-7);
ss) α,α,5-trifluoro-3-pyridinepropanoic acid (CAS 2138272-98-5);
tt) 5-(difluoromethyl)-3-pyridinepropanoic acid (CAS 1785567-84-1);
uu) 6-(difluoromethyl)-3-pyridinepropanoic acid (CAS 1784836-51-6);
vv) 2-(difluoromethyl)-3-pyridinepropanoic acid (CAS 1780289-79-3);
ww) 4-(trifluoromethyl)-3-pyridinepropanoic acid (CAS 1603111-54-1);
xx) 5-fluoro-3-pyridinebutanoic acid (CAS 1198074-59-7);
yy) 6-(trifluoromethyl)-3-pyridinebutanoic acid (CAS 1100766-80-0);
zz) 6-fluoro-3-pyridinepropanoic acid (CAS 944998-15-6);
aaa) 5-(trifluoromethyl)-3-pyridinepropanoic acid (CAS 915030-12-5);
bbb) 6-(trifluoromethyl)-3-pyridinepropanoic acid (CAS 539855-70-4); and
ccc) 5-fluoro-3-pyridinepropanoic acid (CAS 22620-28-6).

Non-limiting examples of starting materials in which X is a substituted 4-pyridinyl ring include:
a) α-3,5-trifluoro-4-pyridinepropanoic acid (CAS 2380978-27-6);
b) α,α,2-trifluoro-4-pyridinepropanoic acid (CAS 2360119-15-7);
c) β,β,2-trifluoro-4-pyridinepropanoic acid (CAS 2359059-42-8);
d) 2,3-difluoro-4-pyridinebutanoic acid (CAS 2358586-78-2);
e) β,β,2,3-tetrafluoro-4-pyridinepropanoic acid (CAS 2358473-56-8);
f) 3-(trifluoromethyl)-4-pyridinebutanoic acid (CAS 2357880-42-1);
g) α,α,2,3-tetrafluoro-4-pyridinepropanoic acid (CAS 2355001-55-5);
h) 2-fluoro-4-pyridinebutanoic acid (CAS 2354443-11-9);
i) 2,3-difluoro-4-pyridinepropanoic acid (CAS 2354302-13-7);
j) 2-fluoro-4-pyridinepropanoic acid (CAS 2352744-10-4);
k) 3,5-difluoro-4-pyridinepentanoic acid (CAS 2285017-33-4);
l) 3-fluoro-4-pyridinehexanoic acid (CAS 2229808-55-1);
m) β,β,3,5-tetrafluoro-4-pyridinepropanoic acid (CAS 2229498-88-6);
n) α,α-difluoro-3-(trifluoromethyl)-4-pyridinepropanoic acid (CAS 2229097-30-5);
o) β,β,3-trifluoro-4-pyridinepropanoic acid (CAS 2228730-46-7);
p) β,β-difluoro-3-(trifluoromethyl)-4-pyridinepropanoic acid (CAS 2228606-55-9);
q) 3,5-difluoro-4-pyridinebutanoic acid (CAS 2228325-44-6);
r) 3-fluoro-4-pyridinebutanoic acid (CAS 2228162-89-6);
s) α,3,5-trifluoro-4-pyridinepropanoic acid (CAS 2166862-35-5);
t) α,α,3,5-tetrafluoro-4-pyridinepropanoic acid (CAS 2138554-68-2);
u) α,α,3-trifluoro-4-pyridinepropanoic acid (CAS 2137827-10-0);
v) 3,5-difluoro-4-pyridinepropanoic acid (CAS 1996164-11-4);
w) 3-(trifluoromethyl)-4-pyridinepropanoic acid (CAS 1888850-59-6);
x) 2,5-difluoro-4-pyridinepropanoic acid (CAS 1780779-62-5);
y) 2-(1,1-difluoroethyl)-4-pyridinepropanoic acid (CAS 1780673-65-5);
z) 2-(difluoromethyl)-4-pyridinepropanoic acid (CAS 1780289-72-6);
aa) 3-fluoro-4-pyridinepropanoic acid (CAS 1256819-25-6);
bb) 2,3,5,6-tetrafluoro-4-pyridinepropanoic acid (CAS 916792-08-0);

Non-limiting examples of starting materials in which X is a substituted pyrazine ring include:
a) 6-(difluoromethyl)-2-pyrazinepropanoic acid (CAS 1780915-43-6);
b) 5-(difluoromethyl)-2-pyrazinepropanoic acid (CAS 1780310-08-8); and
c) 5-(trifluoromethyl)-2-pyrazinepropanoic acid (CAS 1196156-94-1).

Non-limiting examples of starting materials in which X is a substituted pyridazine ring include:
a) 4-(trifluoromethyl)-3-pyridazineacetic acid (CAS 1898213-83-6);
b) 6-(trifluoromethyl)-3-pyridazineacetic acid (CAS 1565408-90-3);
c) 6-(difluoromethyl)-3-pyridazineacetic acid (CAS 2303714-09-0);
d) 6-(trifluoromethyl)-3-pyridazineacetic acid (CAS 1898214-51-1); and
e) 5-(trifluoromethyl)-3-pyridazineacetic acid (CAS 1898213-96-1).

The substitution patterns in the groups above are non-limiting examples of the substitution patterns for R₁, R₂, R₃, R₄, R₅, R₆, and R₇, regardless of the ring to which R₁, R₂, R₃, R₄, and R₅ are bound or the length of the carbon chain to which R₆ and R₇ are bound.

### Examples

### I. Methods and Materials

### 1. General

All reagents and solvents were purchased from Sigma-Aldrich, Chem-Impex International and ThermoFisher, and were used directly without further purification. The peptide RGD and FRGD were purchased from Peptides International, and CPC Scientific Inc., respectively. The ⁶⁴CuCl₂ was purchased from Washington University School of Medicine in St. Louis and the ¹¹¹InCl₃ was purchased from Jubilant DraxImage Radiopharmacies, Inc. (Triad Isotopes).

### 2. Synthesis of Portable ABX Moieties

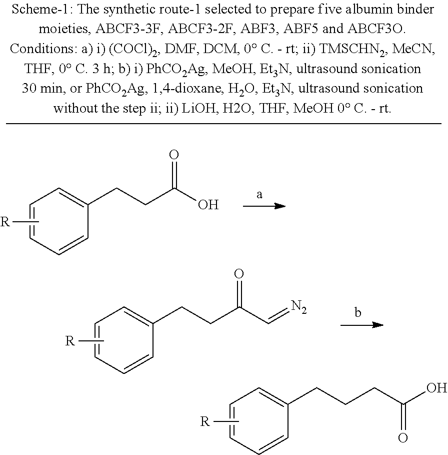

### a. Preparing 4-(3,4,5-trifluorophenyl)butanoic acid (ABF3)

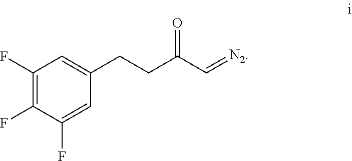

### 1-diazo-4-(3,4,5-trifluorophenyl)butan-2-one

To a stirred solution of 3-(3,4,5-trifluorophenyl)propanoic acid (204 mg, 1.0 mmol) in dry DCM (5 mL) at 0° C., oxalyl chloride (128 μL, 1.5 mmol) was added dropwise; then the mixture was warmed up to room temperature and stirred for another 2 hours. The solvent and extra oxalyl chloride was removed by vacuum, and then THF (2.5 mL) and MeCN (2.5 mL) were added. The mixture was re-cooled down to 0° C., and TMSCHN₂ (1.5 mL, 3 mmol) was added dropwise followed by warming up to room temp gradually and stirred for 3 hours. The resulting solution was diluted with Et₂O (50 mL) and washed sequentially with 0.1 M citric acid, NaHCO3 sat aq. and brine, the organic phase was dried over Na₂SO₄ and purified by silica gel with Hexane:EA=2:1 (Rf=0.15). The product was obtained 204 mg as light-yellow oil, yield 89%.

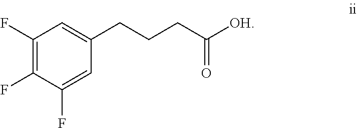 [SMILES: O=C(O)CCCc1cc(F)c(F)c(F)c1]

### 4-(3,4,5-trifluorophenyl)butanoic acid (ABF3)

A mixture of 1-diazo-4-(3,4,5-trifluorophenyl)butan-2-one (91 mg, 0.4 mmol), PhCO₂Ag (9 mg, 0.04 mmol) in Dioxane (3.2 mL) and water (0.6 mL) was sonicated for 4 hours. Then the resulting mixture was acidified by 1N HCl to pH=4 and extracted by EtOAc. The organic phase was washed by brine, dried over anhydrous sodium sulfate and purified by column chromatograph with DCM/MeOH 20:1. 33 mg of product was obtained as yellow oil, yield 38%. ¹H NMR (400 MHz, CDCl₃) δ 6.86-6.74 (m, 2H), 2.67-2.58 (m, 2H), 2.38 (t, J=7.3 Hz, 2H), 1.98-1.88 (m, 2H).

### b. Preparing 4-(2-fluoro-4-(trifluoromethyl)phenyl)butanoic acid (ABCF3-2F)

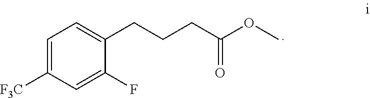 [SMILES: COC(=O)CCCc1ccc(C(F)(F)F)cc1F]

### Methyl 4-(2-fluoro-4-(trifluoromethyl)phenyl)butanoate

A mixture of 1-diazo-4-(2-fluoro-4-(trifluoromethyl)phenyl)butan-2-one (50 mg, 0.19 mmol), PhCO₂Ag (8.8 mg, 0.04 mmol) and Et₃N (0.12 mL) in MeOH (1.9 mL) was sonicated for 30 min. After removal of the solvent by vacuum, the residue was re-dissolved in EtOAc (50 mL), and washed with 0.1 M citric acid, sodium bicarbonate sat aq., brine and dried over anhydrous sodium sulfate. The crude was purified by column chromatography on silica gel with Hexane/EtOAc 4:1 (Rf 0.3). 18 mg product was obtained as colorless oil, yield 36%. ¹H NMR (400 MHz, CDCl₃) δ 7.36-7.27 (m, 3H), 7.12-6.99 (m, 2H), 3.67 (s, 3H), 2.74 (t, J=7.5 Hz, 2H), 2.36 (t, J=7.4 Hz, 2H), 1.97 (p, J=7.4 Hz, 2H).

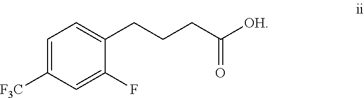 [SMILES: O=C(O)CCCc1ccc(C(F)(F)F)cc1F]

### 4-(2-fluoro-4-(trifluoromethyl)phenyl)butanoic acid (ABCF3-2F)

A mixture of Methyl 4-(2-fluoro-4-(trifluoromethyl)phenyl)butanoate (18 mg, 0.07 mmol), LiOH·H2O (14 mg, 0.34 mmol) in H₂O (0.34 mL), THF (0.34 mL) and MeOH (0.02 mL) was stirred for 2 hours. The resulting mixture was diluted with EtOAc (50 mL) and acidified by 1N HCl. The organic phase was partitioned and washed with brine, dried over anhydrous sodium sulfate. After removal of the solvent the crude was directly used in next step without further purification. 16 mg crude was obtained as yellow oil, yield 94%. ESI-TOF, C₁₁H₁₀F₄O₂, [M−H]⁻ calcd 249.06, found 249.03.

### c. Preparing 4-(3-fluoro-4-(trifluoromethyl)phenyl)butanoic acid (ABCF3-3F)

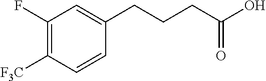 [SMILES: O=C(O)CCCc1ccc(C(F)(F)F)c(F)c1]

### 4-(3-fluoro-4-(trifluoromethyl)phenyl)butanoic acid (ABCF3-3F)

This compound was prepared using the same procedure as ABCF3-2F above, but with 4-(3-fluoro-4-(trifluoromethyl)phenyl) propanoic acid as the starting material. ¹H NMR (400 MHz, CDCl₃) δ 7.51 (t, J=7.7 Hz, 1H), 7.12-6.99 (m, 2H), 2.80-2.67 (m, 2H), 2.40 (t, J=7.3 Hz, 2H), 2.04-1.92 (m, 2H). ESI-TOF, C₁₁H₁₀F₄O₂, [M−H]⁻ calcd 249.06, found 249.05.

### d. Preparing 4-(2-fluoro-4-(trifluoromethyl)phenyl)butanoic acid ABCF3O)

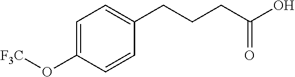 [SMILES: O=C(O)CCCc1ccc(OC(F)(F)F)cc1]

### 4-(4-(trifluoromethoxy)phenyl)butanoic acid (ABCF3O)

This compound was prepared using the same procedure as ABCF3-2F above, but with 4-(4-(trifluoromethoxy)phenyl) propanoic acid as the starting material. ¹H NMR (400 MHz, CDCl₃) δ 7.22-7.16 (m, 2H), 7.15-7.10 (m, 1H), 3.67 (s, 1H), 2.69-2.60 (m, 1H), 2.33 (t, J=7.4 Hz, 1H), 2.00-1.89 (m, 1H).

### e. Preparing 4-(perfluorophenyl)butanoic acid (ABF5)

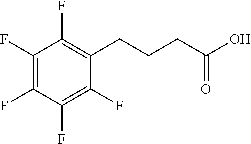 [SMILES: O=C(O)CCCc1c(F)c(F)c(F)c(F)c1F]

### 4-(perfluorophenyl)butanoic acid (ABF5)

This compound was prepared using the same procedure as ABCF3-2F above, but with 4-(perfluorophenyl) propanoic acid as the starting material. ¹H NMR (400 MHz, CDCl₃) δ 2.78 (t, J=7.6 Hz, 2H), 2.42 (t, J=7.4 Hz, 2H), 1.94 (p, J=7.5 Hz, 2H).

### f. Preparing 4-(3,5-difluoro-4-(trifluoromethyl)phenyl)butanoic acid (ABCF3-3,5F)

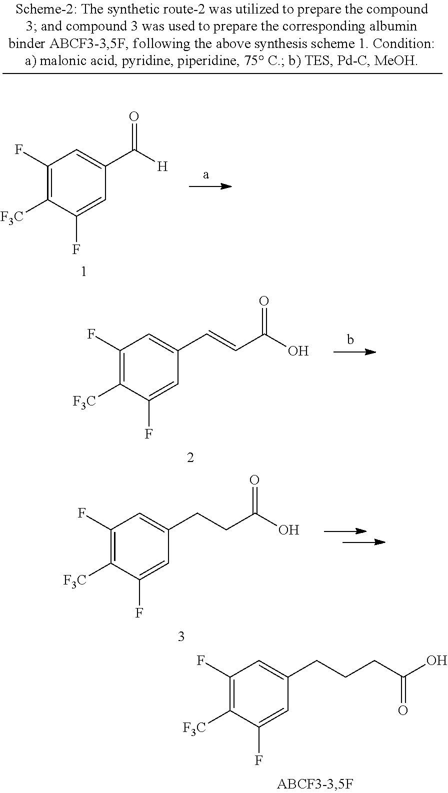 [SMILES: O=Cc1cc(F)c(C(F)(F)F)c(F)c1 ; O=C(O)/C=C/c1cc(F)c(C(F)(F)F)c(F)c1 ; O=C(O)CCc1cc(F)c(C(F)(F)F)c(F)c1 ; O=C(O)CCCc1cc(F)c(C(F)(F)F)c(F)c1]

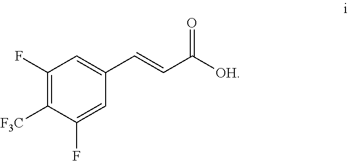 [SMILES: O=C(O)/C=C/c1cc(F)c(C(F)(F)F)c(F)c1]

### (E)-3-(3,5-difluoro-4-(trifluoromethyl)phenyl)acrylic acid

A mixture of 3,5-difluoro-4-(trifluoromethyl)benzaldehyde (940 mg, 447 mmol), malonic acid (990 mg, 9.52 mmol) and piperidine (47 μL) in pyridine (2.5 mL) was heated to 70° C. and stirred for 18 hours. The resulting mixture was poured into 100 mL water and acidified by 1N HCl to adjust pH=4. The formed light-yellow precipitate was filtered, rinsed with water, and collected. The crude was dried under high vacuum to give product 940 mg, yield 67%. ¹H NMR (400 MHz, DMSO) δ 12.79 (br s, 1H), 7.81 (d, J=11.9 Hz, 2H), 7.59 (d, J=16.0 Hz, 1H), 6.83 (d, J=16.0 Hz, 1H).

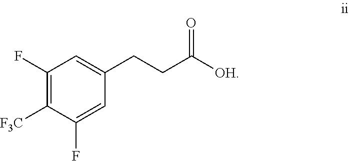 [SMILES: O=C(O)CCc1cc(F)c(C(F)(F)F)c(F)c1]

### 3-(3,5-difluoro-4-(trifluoromethyl)phenyl)propanoic acid

To a stirred solution of (E)-3-(3,5-difluoro-4-(trifluoromethyl)phenyl)acrylic acid (760 mg, 3.0 mmol), Pd—C (10% wt, 76 mg) in MeOH (15 mL) was added TES (5 mL) during 40 min. The resulting mixture was filtered through Celite, concentrated under vacuum and purified column chromatograph on silica gel DCM/MeOH 10:1. The product was obtained 500 mg as colorless oil, yield 71%. ¹H NMR (400 MHz, DMSO) δ 12.26 (s, 1H), 7.31 (d, J=11.5 Hz, 2H), 2.90 t, J=7.6 Hz, 2H), 2.62 (t, J=7.6 Hz, 2H).

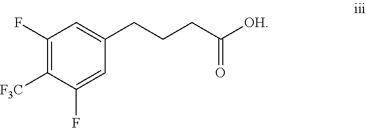 [SMILES: O=C(O)CCCc1cc(F)c(C(F)(F)F)c(F)c1]

### 4-(3,5-difluoro-4-(trifluoromethyl)phenyl)butanoic acid (ABCF3-3,5F)

¹H NMR (400 MHz, CDCl₃) δ 6.86-6.74 (m, 2H), 2.67-2.58 (m, 2H), 2.38 (t, J=7.3 Hz, 2H), 1.98-1.88 (m, 2H).

### g. Preparing 4-(4-fluoronaphthalen-2-yl)butanoic acid (ABNaphth-4F)

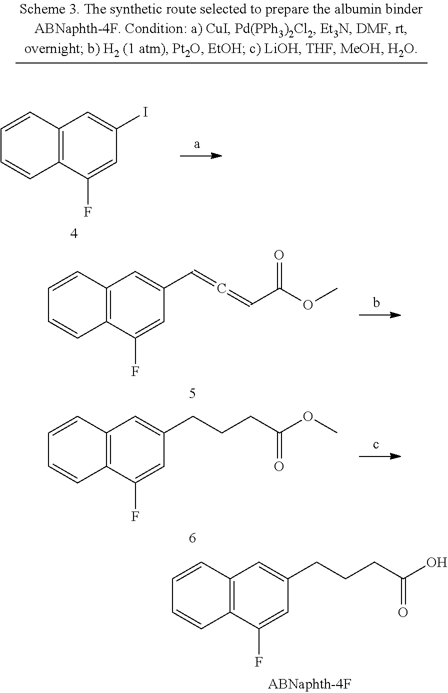

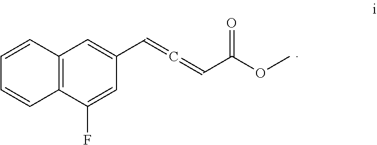 [SMILES: COC(=O)C=C=Cc1cc(F)c2ccccc2c1]

### Methyl 4-(4-fluoronaphthalen-2-yl)buta-2,3-dienoate

A mixture of 1-fluoro-3-iodonaphthalene (54 mg, 0.2 mmol), methyl but-3-ynoate (24 mg, 0.24 mmol), Pd(PPh₃)₂Cl₂ (14 mg, 0.02 mmol), CuI (13 mg, 0.07 mmol) and Et3N (0.4 mL) in DMF (2 mL) was stirred under Argon at room temp overnight. The resulting slurry mixture was diluted with EtOAc (50 mL) and washed with water, brine and dried over anhydrous sodium sulfate. The crude was purified by column chromatography on silica gel with Hexanes/EtOAc 8:1 (Rf 0.3) to give product 20 mg as colorless oil, yield 42%. ¹H NMR (400 MHz, CDCl₃) δ 8.24-7.95 (m, 1H), 7.89-7.72 (m, 1H), 7.58-7.44 (m, 3H), 7.11 (dd, J=11.2, 1.3 Hz, 1H), 6.77 (d, J=6.3 Hz, 1H), 6.12 (d, J=6.3 Hz, 1H), 3.79 (s, 3H).

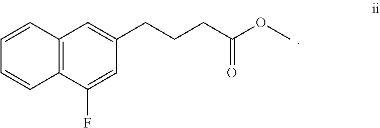 [SMILES: COC(=O)CCCc1cc(F)c2ccccc2c1]

### Methyl 4-(4-fluoronaphthalen-2-yl)butanoate

A mixture of Methyl 4-(4-fluoronaphthalen-2-yl)buta-2,3-dienoate (20 mg, 0.08 mmol), PtO₂ (5 mg) in MeOH (1 mL) was stirred under hydrogen (1 atm) at room temp for 3 hours. The crude was filtered, concentration in vacuum and purified by column chromatography on silica gel with Hexanes/EtOAc 10:1 (Rf 0.3) to give product 20 mg as colorless oil, yield 98%. ¹H NMR (400 MHz, CDCl₃) δ 8.09-8.00 (m, 1H), 7.85-7.72 (m, 1H), 7.53-7.44 (m, 2H), 7.41 (s, 1H), 7.01 (dd, J=11.5, 1.4 Hz, 1H), 3.67 (s, 3H), 2.80 (t, J=7.5 Hz, 2H), 2.37 (t, J=7.4 Hz, 2H), 2.10-1.98 (m, 2H).

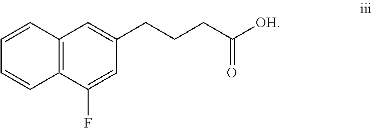 [SMILES: O=C(O)CCCc1cc(F)c2ccccc2c1]

### 4-(4-fluoronaphthalen-2-yl)butanoic acid (ABNaphth-4F)

A mixture of Methyl 4-(4-fluoronaphthalen-2-yl)butanoate (20 mg, 0.08 mmol), LiOH·H2O (17 mg, 0.4 mmol) in H₂O (0.45 mL), THF (0.45 mL) and MeOH (0.1 mL) was stirred for 2 hours. The resulting mixture was diluted with EtOAc (50 mL) and acidified by 1N HCl. The organic phase was partitioned and washed with brine, dried over anhydrous sodium sulfate. After removal of the solvent the crude was directly used in next step without further purification. 17 mg crude was obtained as yellow oil, yield 92%.

### 3. Synthesis of DOTA-Albumin Binder Conjugates for In Vitro Evaluation

### a. Preparing the Intermediate (ABX-NHS)

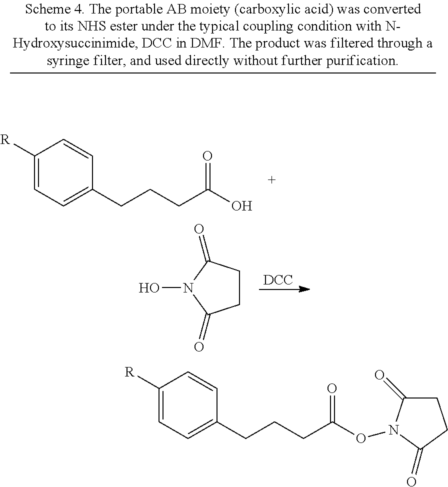

### b. Conjugating DOTA to the Albumin Binder

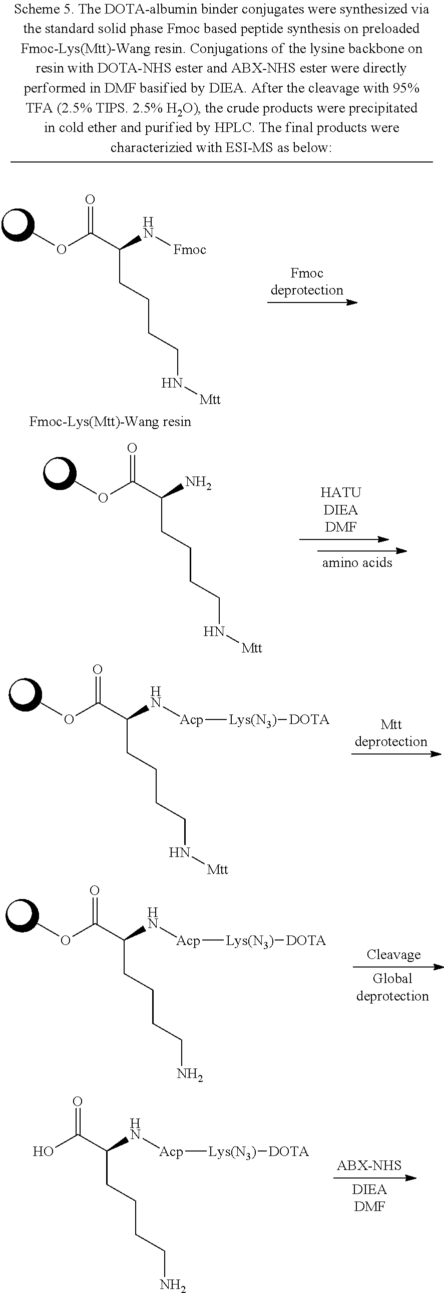

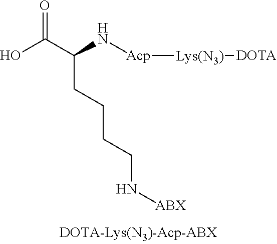

### DOTA-Lys(Azide)-Acp-ABCF3, [M+H]⁺ m/z: 1014.8

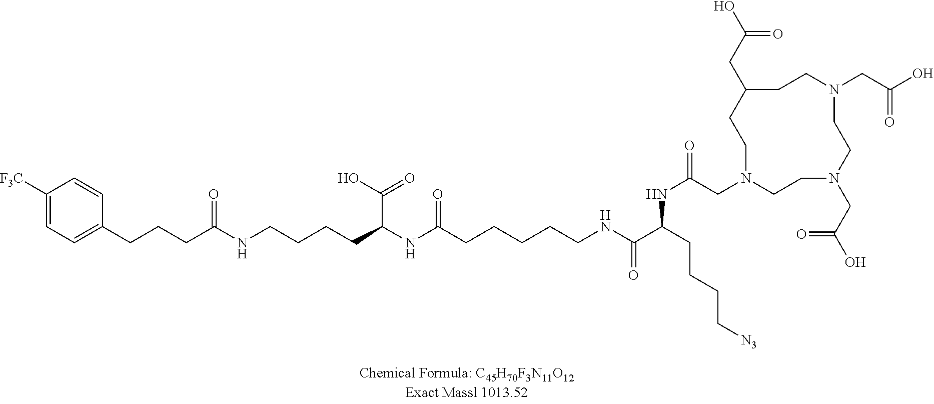 [SMILES: [N-]=[N+]=NCCCC[C@H](NC(=O)CN1CCC(CC(=O)O)CCN(CC(=O)O)CCN(CC(=O)O)CC1)C(=O)NCCCCCC(=O)N[C@@H](CCCCNC(=O)CCCc1ccc(C(F)(F)F)cc1)C(=O)O]

### DOTA-Lys(Azide)-Acp-D-ABCF3, [M+H]⁺ m/z: 1014.7

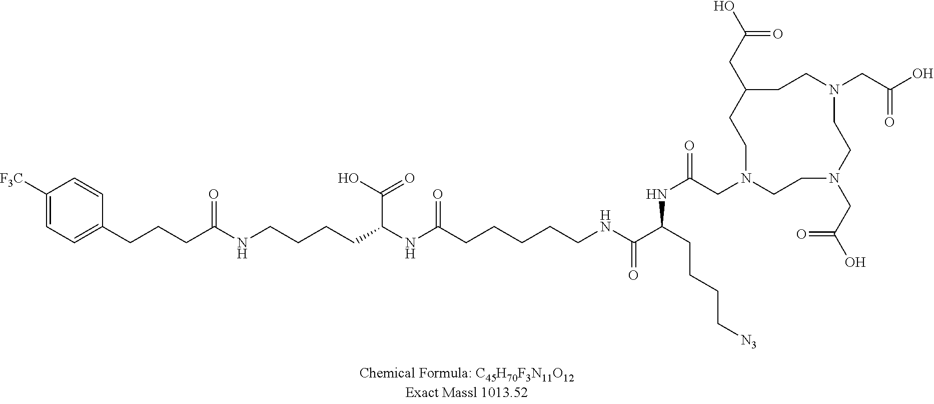 [SMILES: [N-]=[N+]=NCCCC[C@H](NC(=O)CN1CCC(CC(=O)O)CCN(CC(=O)O)CCN(CC(=O)O)CC1)C(=O)NCCCCCC(=O)N[C@H](CCCCNC(=O)CCCc1ccc(C(F)(F)F)cc1)C(=O)O]

### DOTA-Lys(Azide)-Acp-ABCF3-2F, [M+H]⁺ m/z: 1032.4

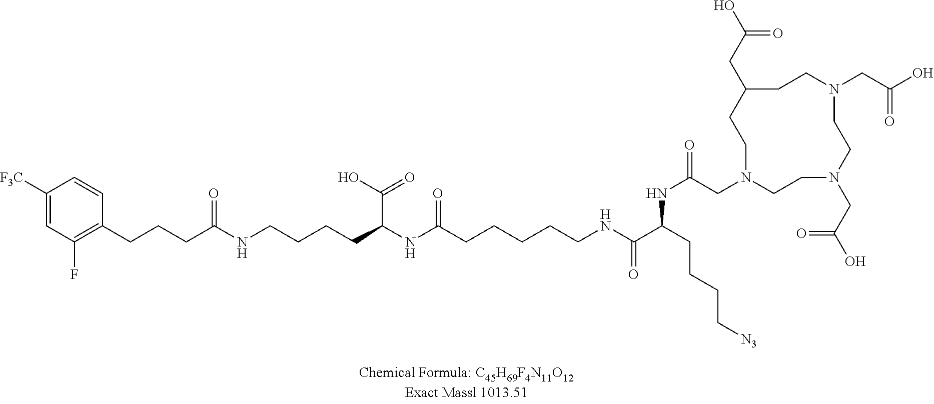 [SMILES: [N-]=[N+]=NCCCC[C@H](NC(=O)CN1CCC(CC(=O)O)CCN(CC(=O)O)CCN(CC(=O)O)CC1)C(=O)NCCCCCC(=O)N[C@@H](CCCCNC(=O)CCCc1ccc(C(F)(F)F)cc1F)C(=O)O]

### DOTA-Lys(Azide)-Acp-ABCF3-3F, [M+H]⁺ m/z: 1032.4

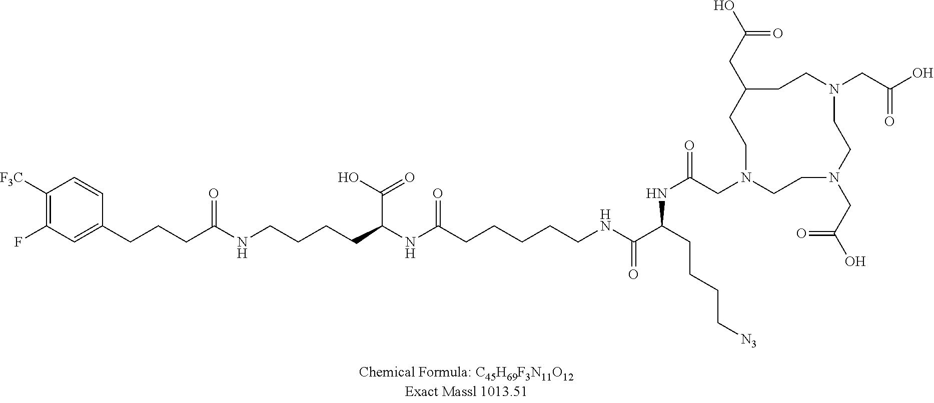 [SMILES: [N-]=[N+]=NCCCC[C@H](NC(=O)CN1CCC(CC(=O)O)CCN(CC(=O)O)CCN(CC(=O)O)CC1)C(=O)NCCCCCC(=O)N[C@@H](CCCCNC(=O)CCCc1ccc(C(F)(F)F)c(F)c1)C(=O)O]

### DOTA-Lys(Azide)-Acp-ABCF3-3,5F, [M+H]⁺ m/z: 1050.2

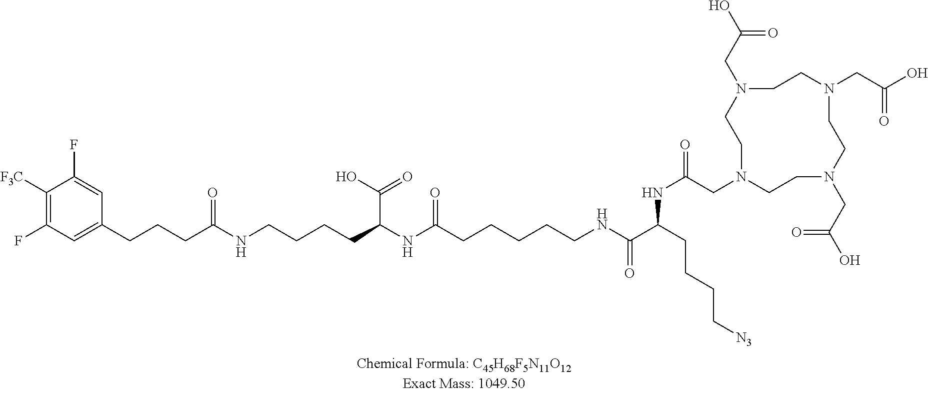 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCCc1cc(F)c(C(F)(F)F)c(F)c1)C(=O)O]

### DOTA-Lys(Azide)-Acp-ABCF3O, [M+H]⁺ m/z: 1028.9

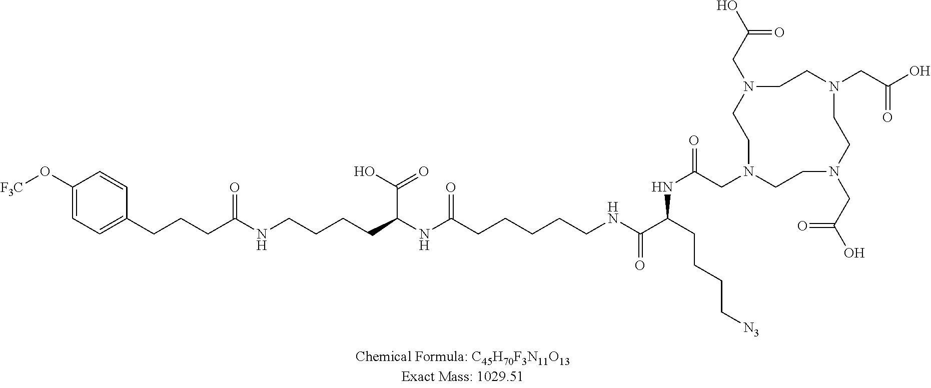 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCCc1ccc(OC(F)(F)F)cc1)C(=O)O]

### DOTA-Lys(Azide)-Acp-ABCF3P, [M+H]⁺ m/z: 1000.4

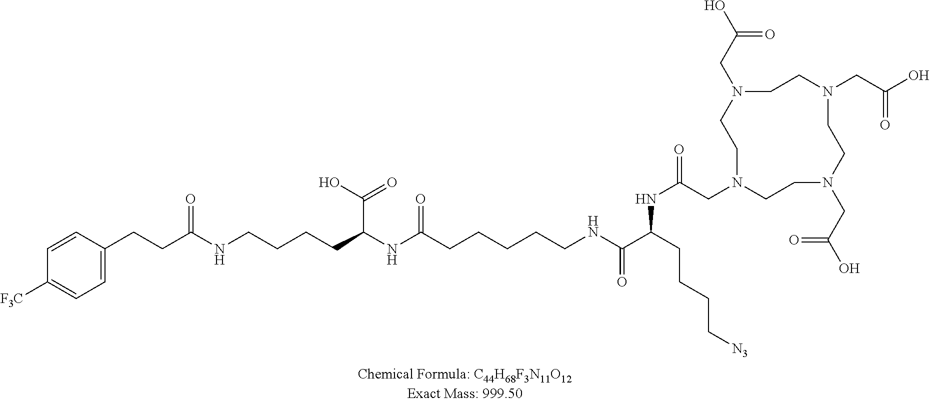 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCc1ccc(C(F)(F)F)cc1)C(=O)O]

### DOTA-Lys(Azide)-Acp-ABF, [M+H]⁺ m/z: 964.8

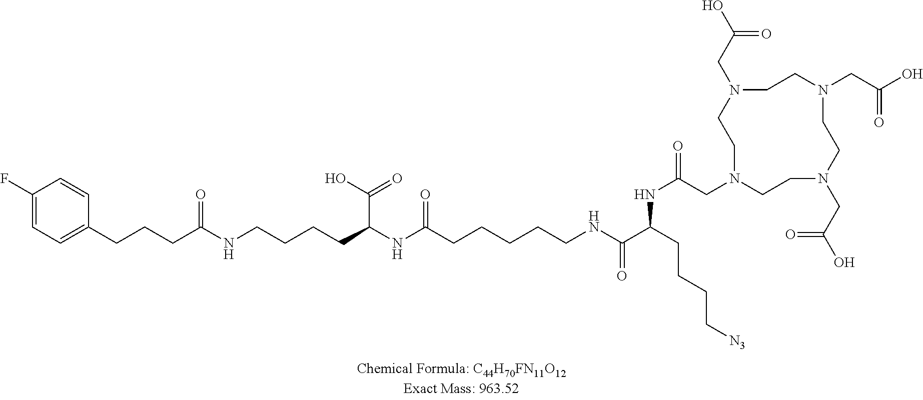 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCCc1ccc(F)cc1)C(=O)O]

### DOTA-Lys(Azide)-Acp-ABF3P, [M+H]⁺ m/z: 986.8

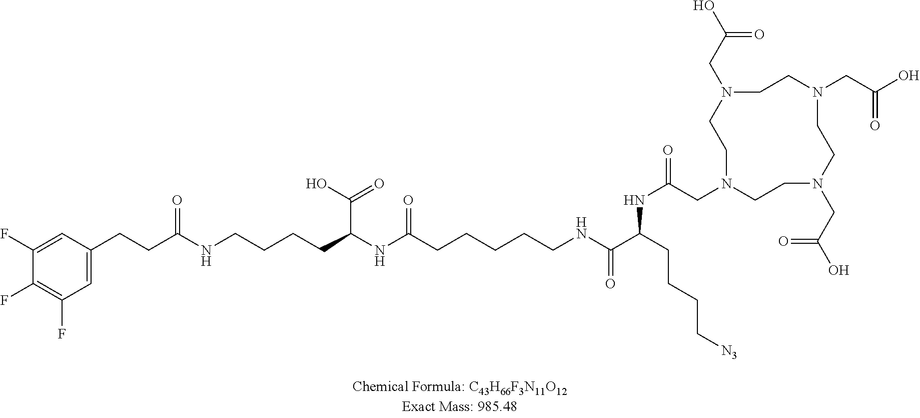 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCc1cc(F)c(F)c(F)c1)C(=O)O]

### DOTA-Lys(Azide)-Acp-ABF5, [M+H]⁺ m/z: 1036.6

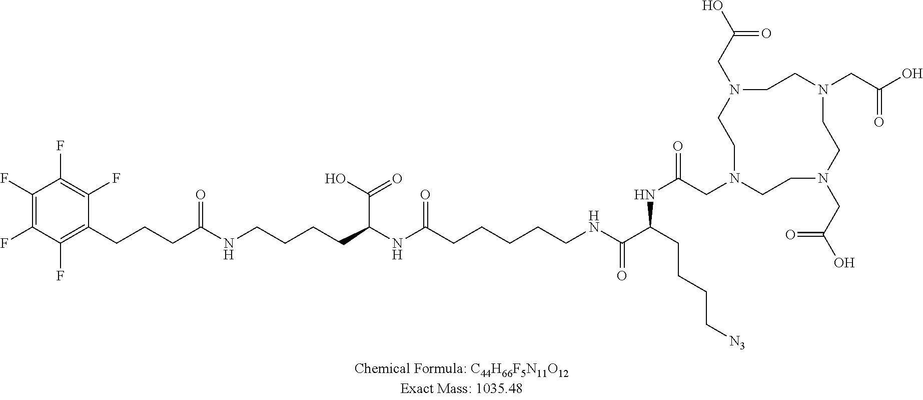 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCCc1c(F)c(F)c(F)c(F)c1F)C(=O)O]

### DOTA-Lys(Azide)-Acp-ABmCF3, [M+H]⁺ m/z: 1014.8

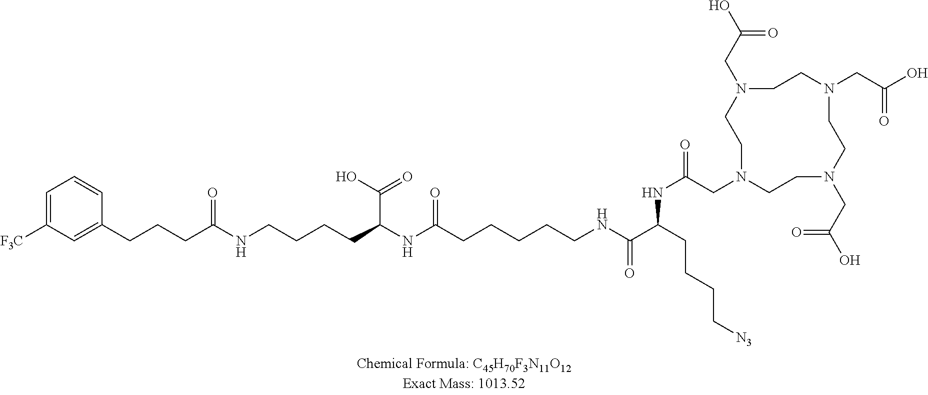 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCCc1cccc(C(F)(F)F)c1)C(=O)O]

### DOTA-Lys(Azide)-Acp-ABNaphth, [M+H]⁺ m/z: 996.8

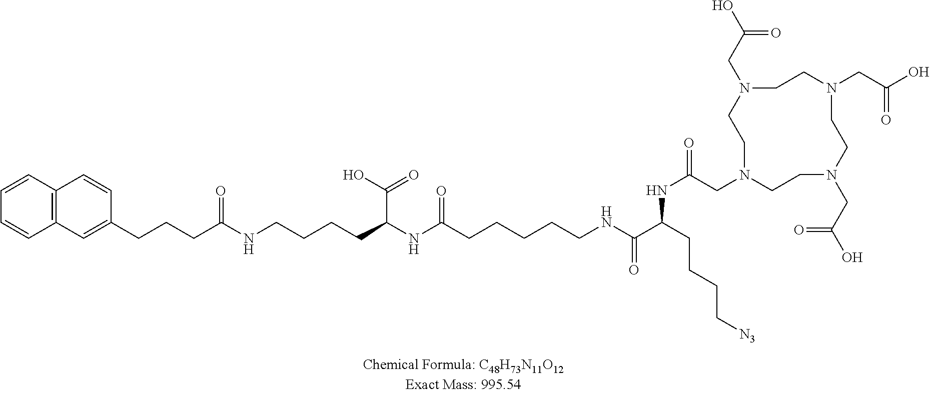 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCCc1ccc2ccccc2c1)C(=O)O]

### DOTA-Lys(Azide)-Acp-ABNaphth-4F, [M+H]⁺ m/z: 1014.3

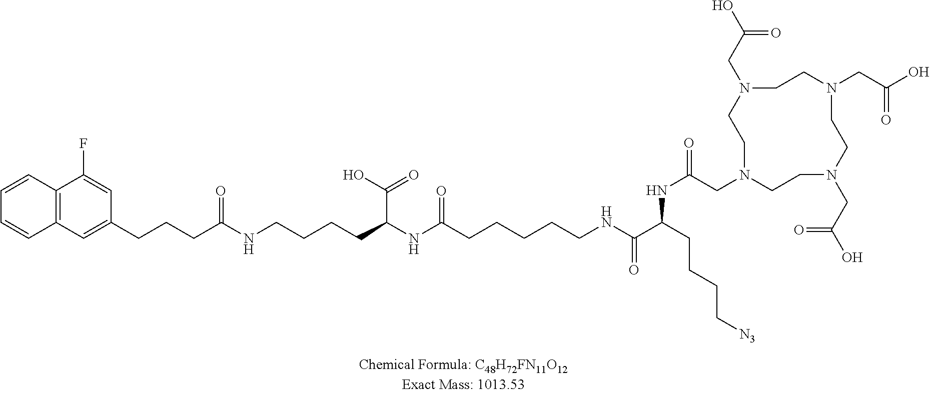 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCCc1cc(F)c2ccccc2c1)C(=O)O]

### DOTA-Lys(Azide)-Acp-ABOCF3, [M+H]⁺ m/z: 1028.6

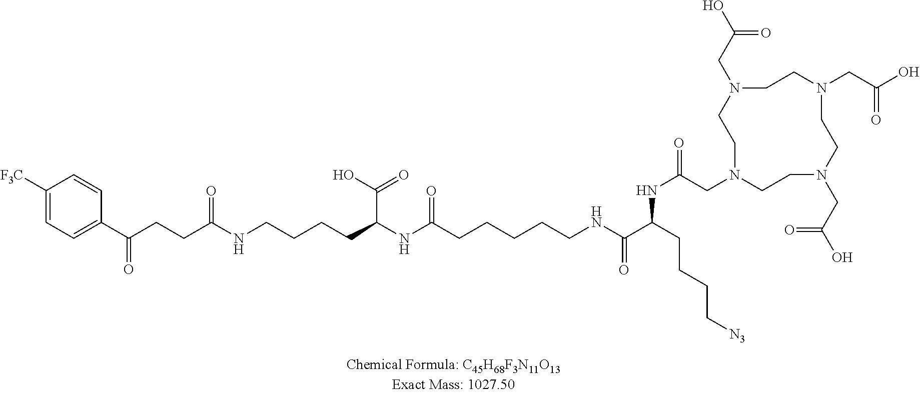 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@@H](CCCCNC(=O)CCC(=O)c1ccc(C(F)(F)F)cc1)C(=O)O]

### DOTA-Lys(Azide)-Acp-hLys-ABF3P, [M+H]⁺ m/z: 1000.6

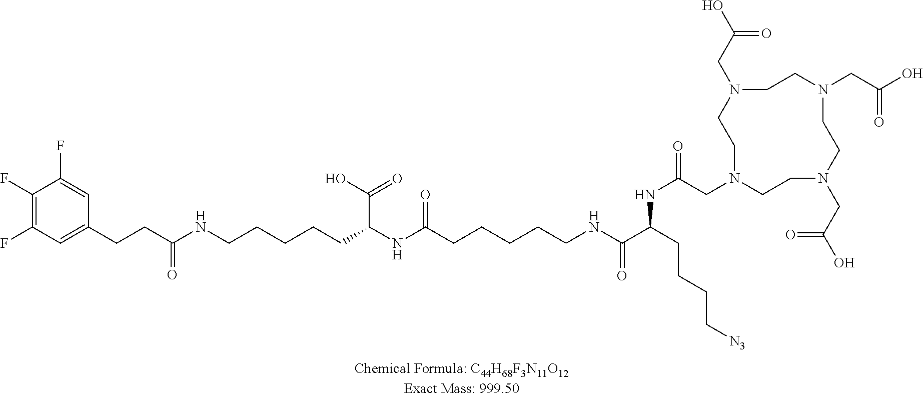 [SMILES: [N-]=[N+]=NCCCC[C@H](NC(=O)CN1CCN(CC(=O)O)CCN(CC(=O)O)CCN(CC(=O)O)CC1)C(=O)NCCCCCC(=O)N[C@H](CCCCCNC(=O)CCc1cc(F)c(F)c(F)c1)C(=O)O]

### DOTA-Lys(Azide)-Gly-εLys-βAla-ABCF3, [M+H]⁺ m/z: 1015.6

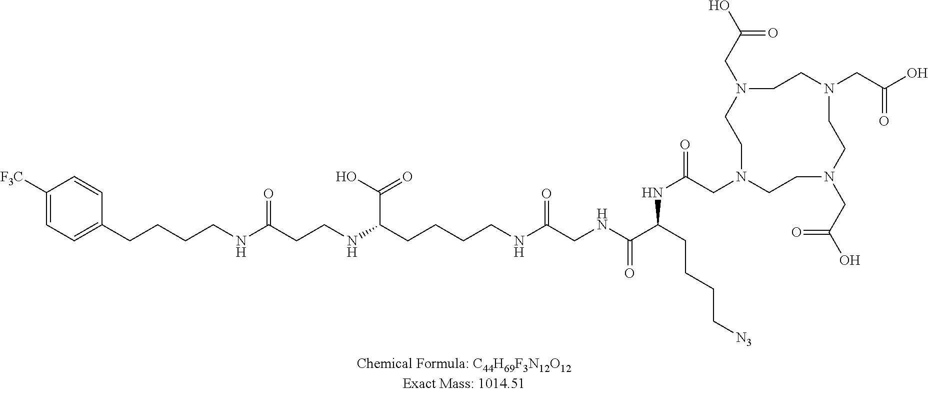 [SMILES: [N-]=[N+]=NCCCC[C@H](NC(=O)CN1CCN(CC(=O)O)CCN(CC(=O)O)CCN(CC(=O)O)CC1)C(=O)NCC(=O)NCCCC[C@H](NCCC(=O)NCCCCc1ccc(C(F)(F)F)cc1)C(=O)O]

### 4. Synthesis and Albumin Binder and Peptide Conjugates

### a. Synthesis of DOTA-RGD-ABX and DOTA-FRGD-ABX (ABX=ABCF3, ABI)

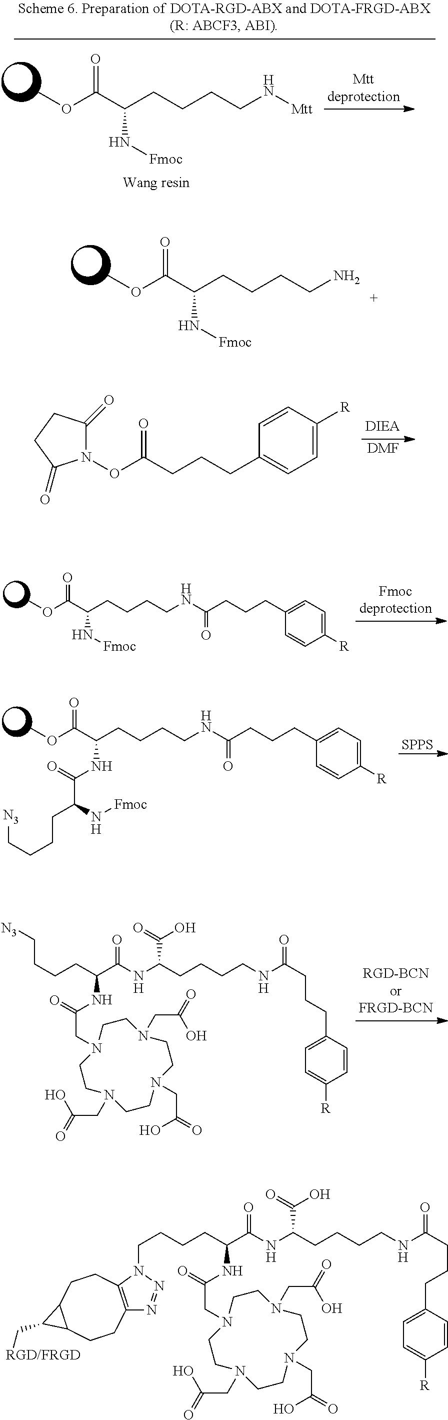

### i. RGD-BCN and FRGD-BCN Preparation

The received peptides were directly used without any further purification. The conjugation was achieved by direct coupling of endo-BCN-PEG4-PFP ester with 1.2× peptide ligand and 4×DIEA in DMF. The resulting products were used for next step without further purification.

### ii. Conjugation of RGD-BCN, FRGD-BCN to DOTA-Lys(N3)-ABCF3/ABI

The final conjugates were obtained from the SPAAC click reaction between the equal ratio of peptide-BCN derivatives and corresponding azide contained chemical tools in 50% acetonitrile and 50% PBS buffer. The products were purified by HPLC, and characterized with ESI-MS as below:

### DOTA-RGD-ABCF3, (R1=CF3), [M+2H]²⁺ M/z: 928.6

### DOTA-RGD-ABI, (R1=I), [M+2H]²⁺ M/z: 957.6

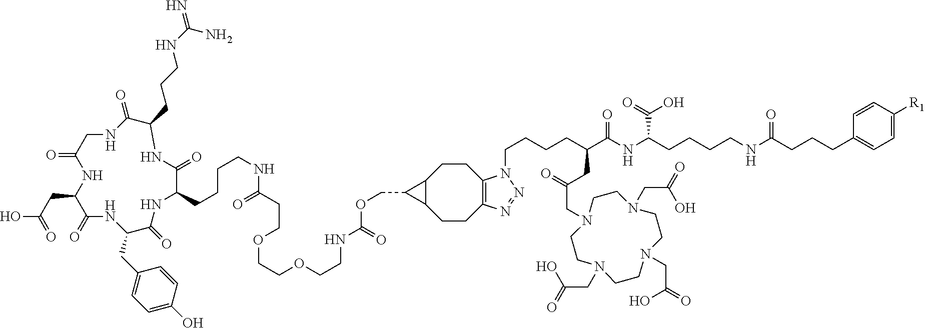

### DOTA-FRGD-ABCF3, (R2=CF3), [M+2H]²⁺ M/z: 1205.9

### DOTA-FRGD-ABI, (R2=I), [M+2H]²⁺ M/z: 1234.7

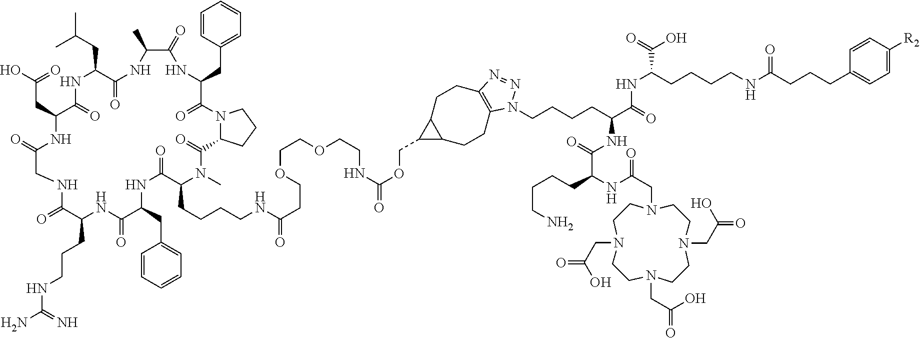

The BCN-SPAAC click reaction utilizing the nitrile on the lysine of the DOTA conjugates provides a non-limiting example of generating a “linker” as the term is used herein. DOTA is a non-limiting example of a radionuclide chelator.

### b. Synthesis of DOTA-SFLAP3-ABX

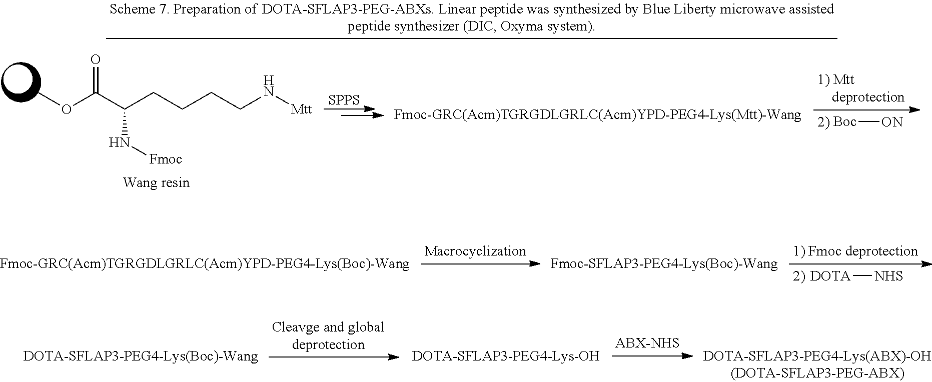

DOTA-PSMA617-ABCF3 were synthesized through the similar strategy. After HPLC purification, the products were characterized with ESI-MS as below:

### DOTA-ABCF3-PSMA617, [M+2H]²⁺ m/z: 807.5

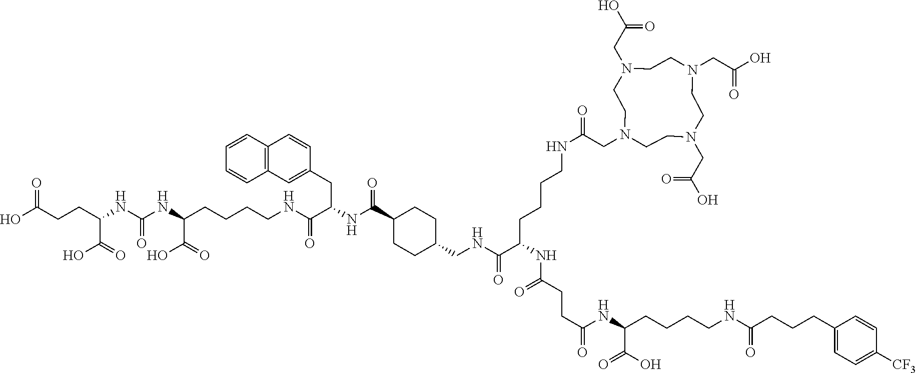 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)[C@H](CCCCNC(=O)CN2CCN(CC(=O)O)CCN(CC(=O)O)CCN(CC(=O)O)CC2)NC(=O)CCC(=O)N[C@@H](CCCCNC(=O)CCCc2ccc(C(F)(F)F)cc2)C(=O)O)CC1)C(=O)O)C(=O)O]

### DOTA-SFLAP3-ABCF3, [M+2H]²⁺ m/z: 1350.5

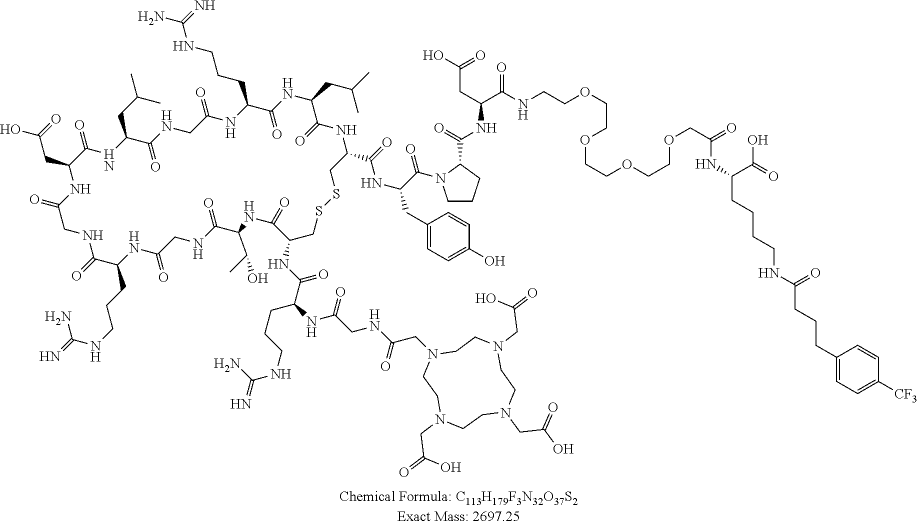 [SMILES: CC(C)C[C@@H]1NC(=O)[C@H](CC(=O)O)NC(=O)CNC(=O)[C@H](CCCNC(=N)N)NC(=O)CNC(=O)[C@H]([C@@H](C)O)NC(=O)[C@@H](NC(=O)[C@H](CCCNC(=N)N)NC(=O)CNC(=O)CN2CCN(CC(=O)O)CCN(CC(=O)O)CCN(CC(=O)O)CC2)CSSC[C@@H](C(=O)N[C@@H](Cc2ccc(O)cc2)C(=O)N2CCC[C@H]2C(=O)N[C@@H](CC(=O)O)C(=O)NCCOCCOCCOCCOCC(=O)N[C@@H](CCCCNC(=O)CCCc2ccc(C(F)(F)F)cc2)C(=O)O)NC(=O)[C@H](CC(C)C)NC(=O)[C@H](CCCNC(=N)N)NC(=O)CNC1=O]

### DOTA-SFLAP3-ABCF3-2F, [M+3H]³⁺ m/z: 906.2

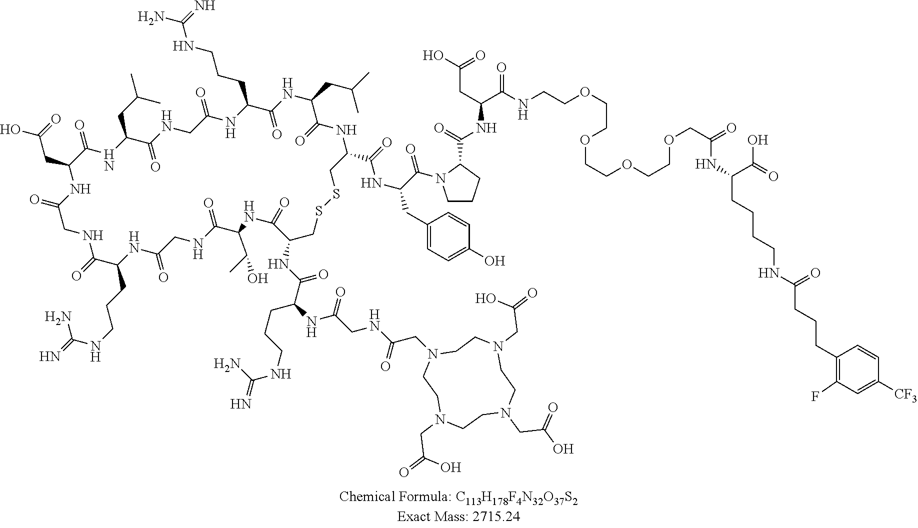 [SMILES: CC(C)C[C@@H]1NC(=O)[C@H](CC(=O)O)NC(=O)CNC(=O)[C@H](CCCNC(=N)N)NC(=O)CNC(=O)[C@H]([C@@H](C)O)NC(=O)[C@@H](NC(=O)[C@H](CCCNC(=N)N)NC(=O)CNC(=O)CN2CCN(CC(=O)O)CCN(CC(=O)O)CCN(CC(=O)O)CC2)CSSC[C@@H](C(=O)N[C@@H](Cc2ccc(O)cc2)C(=O)N2CCC[C@H]2C(=O)N[C@@H](CC(=O)O)C(=O)NCCOCCOCCOCCOCC(=O)N[C@@H](CCCCNC(=O)CCCc2ccc(C(F)(F)F)cc2F)C(=O)O)NC(=O)[C@H](CC(C)C)NC(=O)[C@H](CCCNC(=N)N)NC(=O)CNC1=O]

### DOTA-SFLAP3-ABCF3-2F, [M+2H]²⁺ m/z: 1359.2

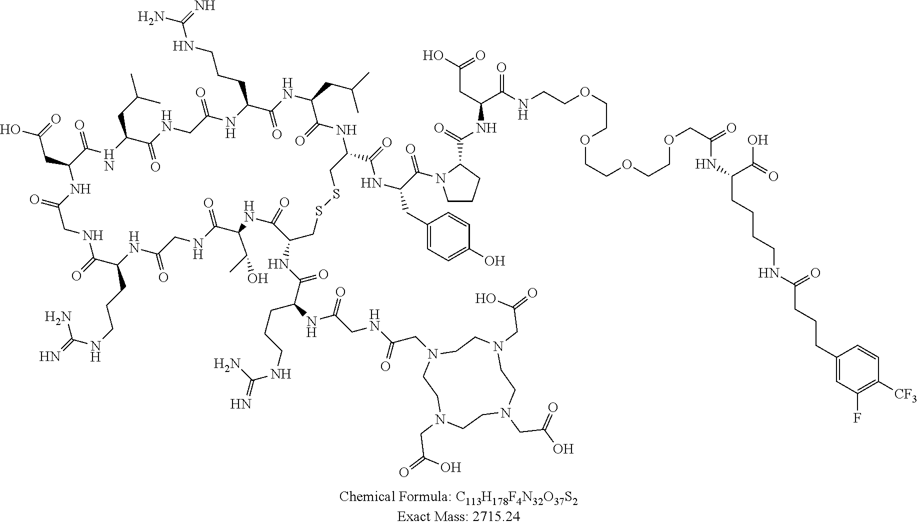 [SMILES: CC(C)C[C@@H]1NC(=O)[C@H](CC(=O)O)NC(=O)CNC(=O)[C@H](CCCNC(=N)N)NC(=O)CNC(=O)[C@H]([C@@H](C)O)NC(=O)[C@@H](NC(=O)[C@H](CCCNC(=N)N)NC(=O)CNC(=O)CN2CCN(CC(=O)O)CCN(CC(=O)O)CCN(CC(=O)O)CC2)CSSC[C@@H](C(=O)N[C@@H](Cc2ccc(O)cc2)C(=O)N2CCC[C@H]2C(=O)N[C@@H](CC(=O)O)C(=O)NCCOCCOCCOCCOCC(=O)N[C@@H](CCCCNC(=O)CCCc2ccc(C(F)(F)F)c(F)c2)C(=O)O)NC(=O)[C@H](CC(C)C)NC(=O)[C@H](CCCNC(=N)N)NC(=O)CNC1=O]

### DOTA-SFLAP3-ABCF3-3,5F, [M+2H]²⁺ m/z: 1367.8

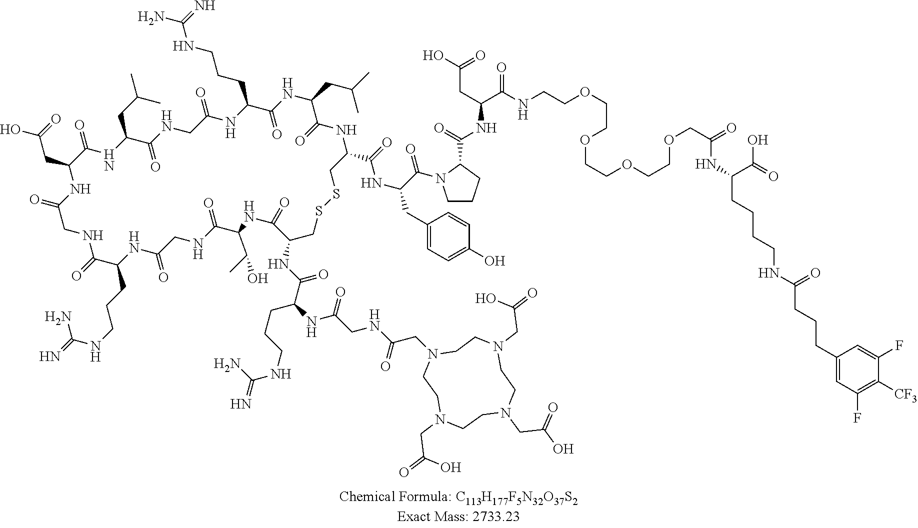 [SMILES: CC(C)C[C@@H]1NC(=O)[C@H](CC(=O)O)NC(=O)CNC(=O)[C@H](CCCNC(=N)N)NC(=O)CNC(=O)[C@H]([C@@H](C)O)NC(=O)[C@@H](NC(=O)[C@H](CCCNC(=N)N)NC(=O)CNC(=O)CN2CCN(CC(=O)O)CCN(CC(=O)O)CCN(CC(=O)O)CC2)CSSC[C@@H](C(=O)N[C@@H](Cc2ccc(O)cc2)C(=O)N2CCC[C@H]2C(=O)N[C@@H](CC(=O)O)C(=O)NCCOCCOCCOCCOCC(=O)N[C@@H](CCCCNC(=O)CCCc2cc(F)c(C(F)(F)F)c(F)c2)C(=O)O)NC(=O)[C@H](CC(C)C)NC(=O)[C@H](CCCNC(=N)N)NC(=O)CNC1=O]

### DOTA-SFLAP3-ABF5, [M+2H]²⁺ m/z: 1360.8

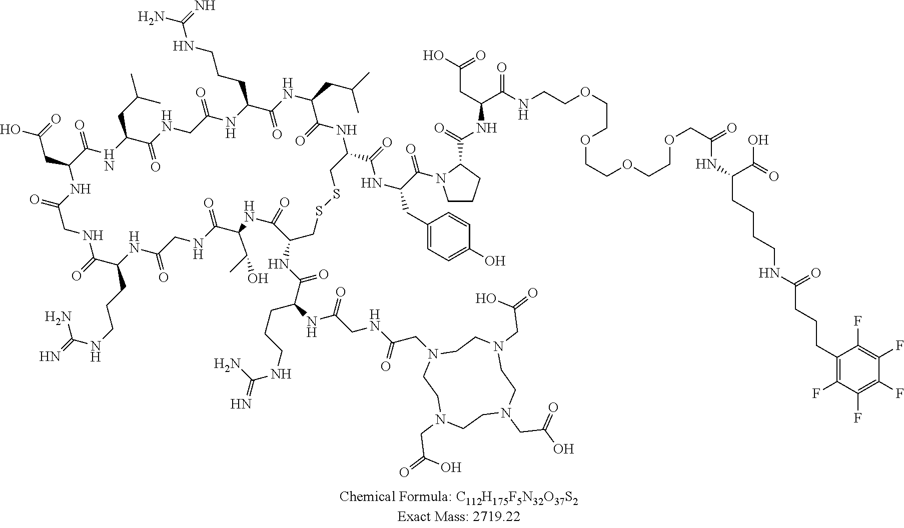 [SMILES: CC(C)C[C@@H]1NC(=O)[C@H](CC(=O)O)NC(=O)CNC(=O)[C@H](CCCNC(=N)N)NC(=O)CNC(=O)[C@H]([C@@H](C)O)NC(=O)[C@@H](NC(=O)[C@H](CCCNC(=N)N)NC(=O)CNC(=O)CN2CCN(CC(=O)O)CCN(CC(=O)O)CCN(CC(=O)O)CC2)CSSC[C@@H](C(=O)N[C@@H](Cc2ccc(O)cc2)C(=O)N2CCC[C@H]2C(=O)N[C@@H](CC(=O)O)C(=O)NCCOCCOCCOCCOCC(=O)N[C@@H](CCCCNC(=O)CCCc2c(F)c(F)c(F)c(F)c2F)C(=O)O)NC(=O)[C@H](CC(C)C)NC(=O)[C@H](CCCNC(=N)N)NC(=O)CNC1=O]

### DOTA-SFLAP3-ABmCF3, [M+2H]²⁺ m/z: 1349.7

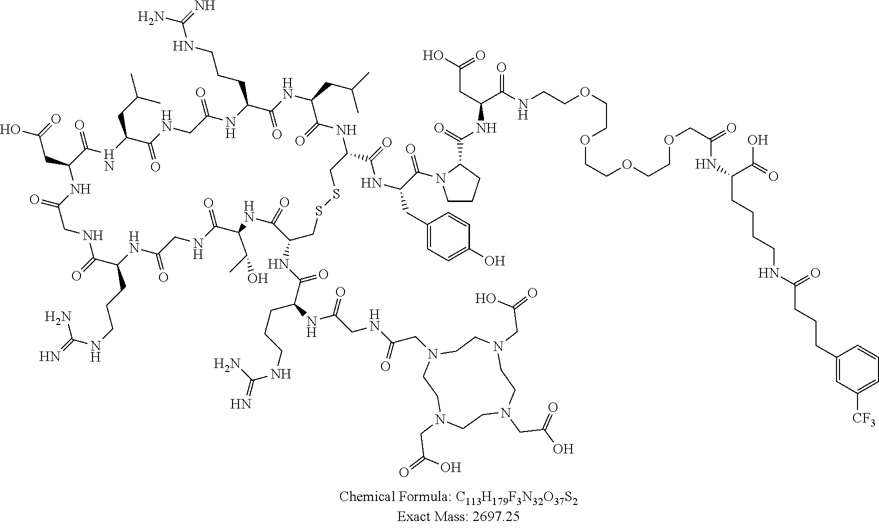 [SMILES: CC(C)C[C@@H]1NC(=O)[C@H](CC(=O)O)NC(=O)CNC(=O)[C@H](CCCNC(=N)N)NC(=O)CNC(=O)[C@H]([C@@H](C)O)NC(=O)[C@@H](NC(=O)[C@H](CCCNC(=N)N)NC(=O)CNC(=O)CN2CCN(CC(=O)O)CCN(CC(=O)O)CCN(CC(=O)O)CC2)CSSC[C@@H](C(=O)N[C@@H](Cc2ccc(O)cc2)C(=O)N2CCC[C@H]2C(=O)N[C@@H](CC(=O)O)C(=O)NCCOCCOCCOCCOCC(=O)N[C@@H](CCCCNC(=O)CCCc2cccc(C(F)(F)F)c2)C(=O)O)NC(=O)[C@H](CC(C)C)NC(=O)[C@H](CCCNC(=N)N)NC(=O)CNC1=O]

### DOTA-SFLAP3-PEG4-ABNaphth, [M+2H]²⁺ m/z: 1340.9

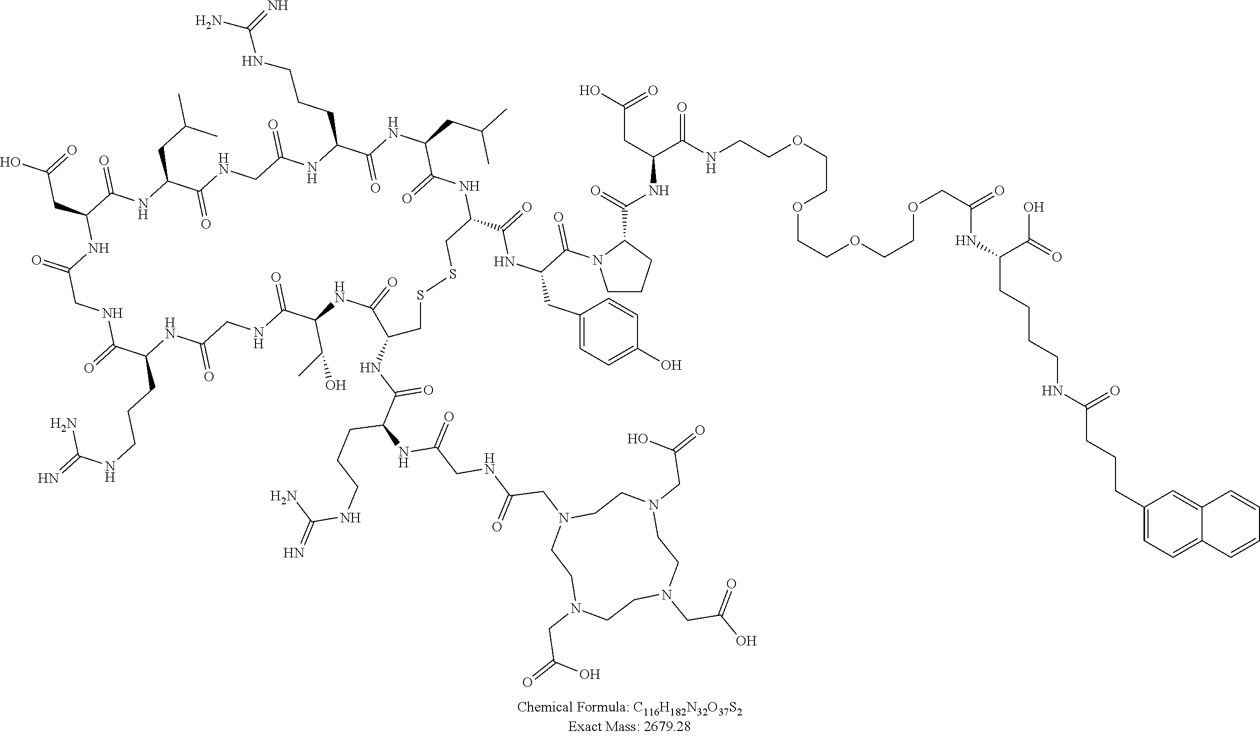 [SMILES: CC(C)C[C@@H]1NC(=O)[C@H](CC(=O)O)NC(=O)CNC(=O)[C@H](CCCNC(=N)N)NC(=O)CNC(=O)[C@H]([C@@H](C)O)NC(=O)[C@@H](NC(=O)[C@H](CCCNC(=N)N)NC(=O)CNC(=O)CN2CCN(CC(=O)O)CCN(CC(=O)O)CCN(CC(=O)O)CC2)CSSC[C@@H](C(=O)N[C@@H](Cc2ccc(O)cc2)C(=O)N2CCC[C@H]2C(=O)N[C@@H](CC(=O)O)C(=O)NCCOCCOCCOCCOCC(=O)N[C@@H](CCCCNC(=O)CCCc2ccc3ccccc3c2)C(=O)O)NC(=O)[C@H](CC(C)C)NC(=O)[C@H](CCCNC(=N)N)NC(=O)CNC1=O]

### DOTA-SFLAP3-ABNaphth-4F, [M+2H]²⁺ m/z: 1349.9

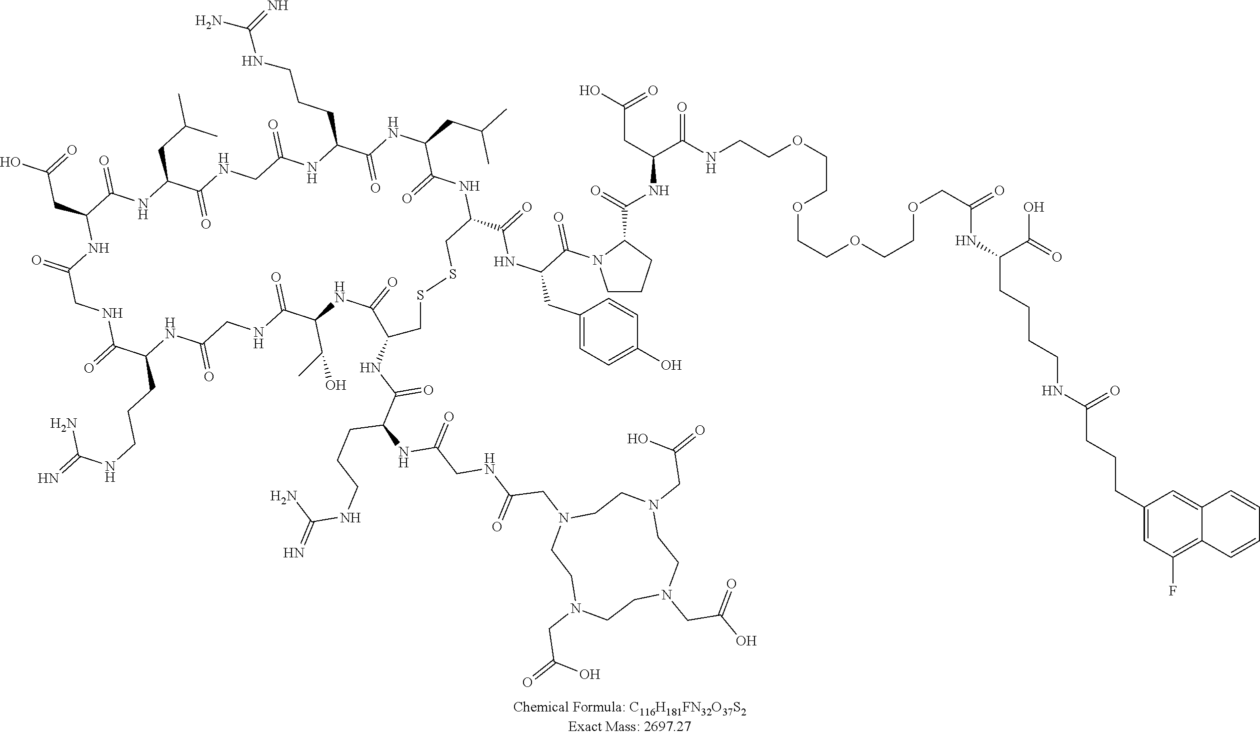 [SMILES: CC(C)C[C@@H]1NC(=O)[C@H](CC(=O)O)NC(=O)CNC(=O)[C@H](CCCNC(=N)N)NC(=O)CNC(=O)[C@H]([C@@H](C)O)NC(=O)[C@@H](NC(=O)[C@H](CCCNC(=N)N)NC(=O)CNC(=O)CN2CCN(CC(=O)O)CCN(CC(=O)O)CCN(CC(=O)O)CC2)CSSC[C@@H](C(=O)N[C@@H](Cc2ccc(O)cc2)C(=O)N2CCC[C@H]2C(=O)N[C@@H](CC(=O)O)C(=O)NCCOCCOCCOCCOCC(=O)N[C@@H](CCCCNC(=O)CCCc2cc(F)c3ccccc3c2)C(=O)O)NC(=O)[C@H](CC(C)C)NC(=O)[C@H](CCCNC(=N)N)NC(=O)CNC1=O]

### 5. Radiolabeling Conditions

For ⁶⁴Cu labeling, the buffer solution was NH₄OAc (0.1 M, pH=7.0). For ¹¹¹In labeling, the buffer solution was NH₄OAc (0.2 M, pH=7.0). All conjugates were labeled at 80° C. for 30 min. The specific activity for ⁶⁴Cu was 18.5 MBq/nmol (500 μCi/nmol) while that for ¹¹¹In was 3.7 MBq/nmol (100 μCi/nmol). The radiochemical purities and the labeling yields were monitored by the reverse phase Radio-HPLC.

### 6. Cell Lines and Animal Models

BxPC3 and CT26 cell lines were purchased from ATCC. The cells were cultured in RPMI media with 10% fetal bovine serum (FBS), 1% penicillin/streptomycin at 37° C. in an atmosphere of 5% CO₂.

### 7. In Vitro Albumin Protein Binding Affinity Assay

Following the published protocol, ultrafiltration assay was performed to assess albumin-binding properties of ¹¹¹In or ⁶⁴Cu labeled albumin binder-radioligand conjugates. ¹¹¹In or ⁶⁴Cu radiolabeling was conducted in NH₄OAc (0.2 M, pH=7.0) buffer at 80° C. for 30 min with a specific activity of 3.7 MBq/nmol (100 μCi/nmol). The resulting albumin binder-radioligand conjugate (10 μL, 10 μM) was mixed with 100 μL human serum albumin (HSA, 1.0 mM in PBS 1×) and 90 μL PBS, followed by shaking gently at 37° C. for 30 min. After the incubation, 80 μL of each solution was loaded on the Zeba™ spin desalting column (7K MWCO) and centrifuged at 1500 g for 2 min. Radioactivity in 20 μL of each filtration and 10 μL mixture (before filtration) were quantified by the γ counter. The filtration rate of each sample was calculated as filtered counts/(mixture counts×2)×100%, all obtained results were then normalized by setting standard ABCF3 moiety as 74.4%. for the comparison purpose. The experiment was performed with triplicates. For working curve, different final concentrations of HSA varied from 10-1500 μM were used, followed the same protocol.

### 8. Biodistribution Studies

Biodistribution studies were performed 2 weeks after the tumor inoculation, when the size of tumor reached 5 mm. ¹¹¹In-labeled albumin binder-radioligand conjugates (1.85 MBq, 100 μL) were then administered via a lateral tail vein. The mice were sacrificed at 1, 2, 16, 24, or 48 h post-injections. Selected organs were collected and weighed, and the radioactivity was measured by the γ counter (Packard, Cobra E5003). The results were calculated as percentage of the injected dose per gram of mass (% ID/g).

### II. Results

### 1. In Vitro Albumin Binding Affinity Assessment

### a. ⁶⁴Cu Radiolabeled DOTA-Albumin Binder Conjugates

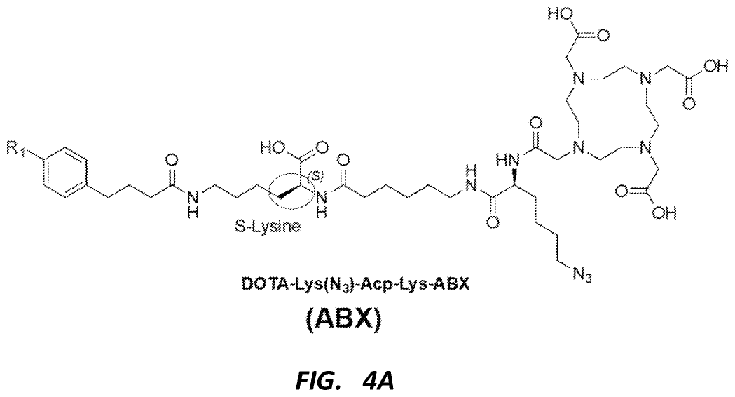

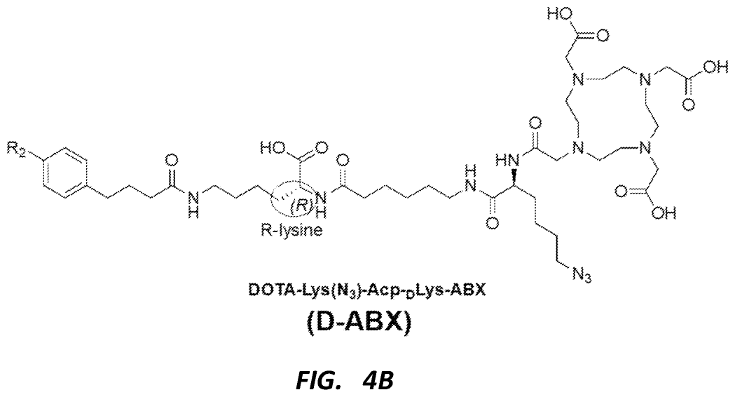

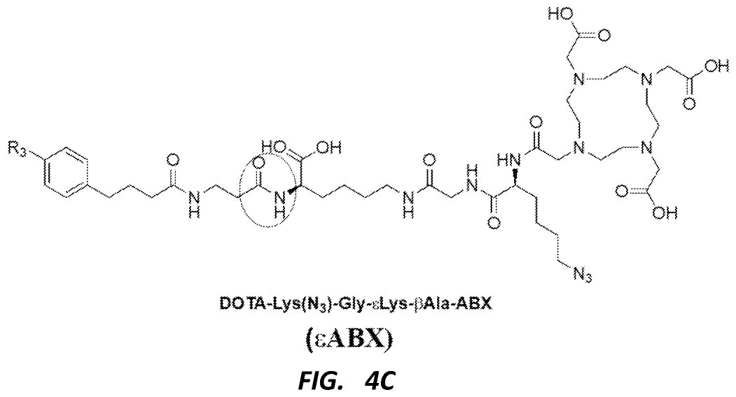

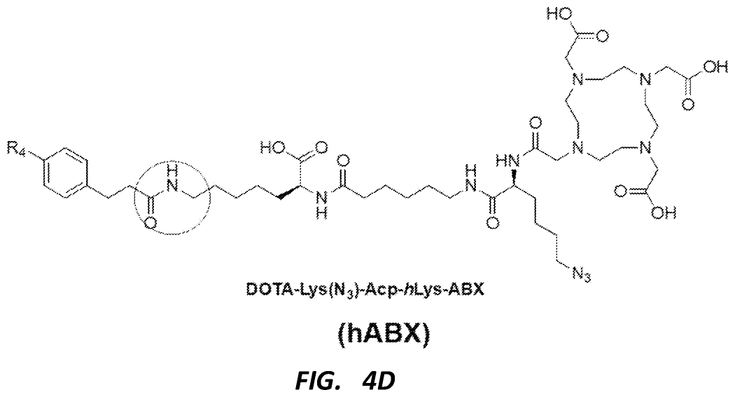

First, the albumin binding affinity of albumin binder via in vitro ultrafiltration assay was assessed DOTA-albumin binder conjugates were prepared for in vitro evaluations based on the general structures shown in FIGS. 4A-4D. (where the X group is represented by an R-substituted benzyl group). These conjugates were prepared to investigate the affinities of the different albumin binding moieties as well as the effects of the following factors: 1) the chiral center of lysine (ABX vs D-ABX in FIGS. 4A and 4B); 2) the additional amide bond between the two binding groups (ABX vs εABX in FIGS. 4A and 4C); 3) the location of amide bond that linked the X group and side-chain of the amino acid (ABX vs hABX in FIGS. 4A and 4D).

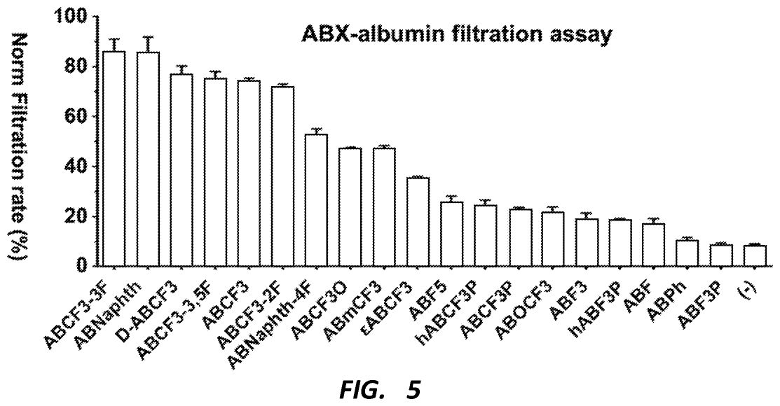

Filtration assay results for all ABXs are shown FIG. 5. [HSA]=500 μM and [tracers]=0.5 μM. All data was normalized, and the filtration percentage for ABCF3 was set up to 75% (average) while DOTA-Lys(N₃)-Acp-Lys-OH was utilized as negative control. In addition to ABX's binding affinities, the filtration assay results also suggested that using D-lysine (D-ABCF3 vs ABCF3) or changing the location of the amide bond (hABCF3P vs ABCF3P, hABF3P vs ABF3P) might have resulted in slightly increased binding affinity, while including an additional amide bond (εABCF3 vs ABCF3) would significantly decrease its binding affinity.

### b. ⁶⁴Cu-DOTA-SFLAP3-PEG4-ABCF3 (Abbreviated as SFLAP3-ABCF3), Compared to ⁶⁴Cu-DOTA-SFLAP3 (Abbreviated as SFLAP3)

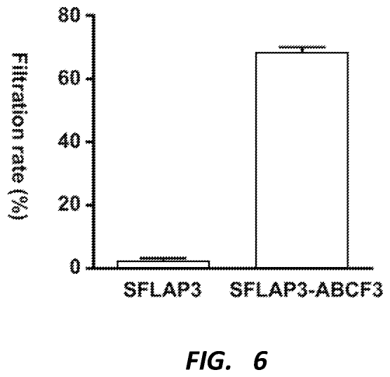

To validate the albumin protein binding of the targeting ligand-incorporated albumin binder, we measured the percentage of SFLAP3-ABCF3/SFLAP3 binding to albumin via filtration assay. As shown in FIG. 6, it was found that ˜75% SFLAP3-ABCF3 bound to albumin protein, while under the same condition only ˜5% SFLAP3 could bind to albumin protein.

### 2. Ex Vivo Biodistribution Studies

### a. ¹¹¹In Labeled RGD-ABCF3, RGD and RGD-ABI in Mice Bearing BxPC3 Xenograft

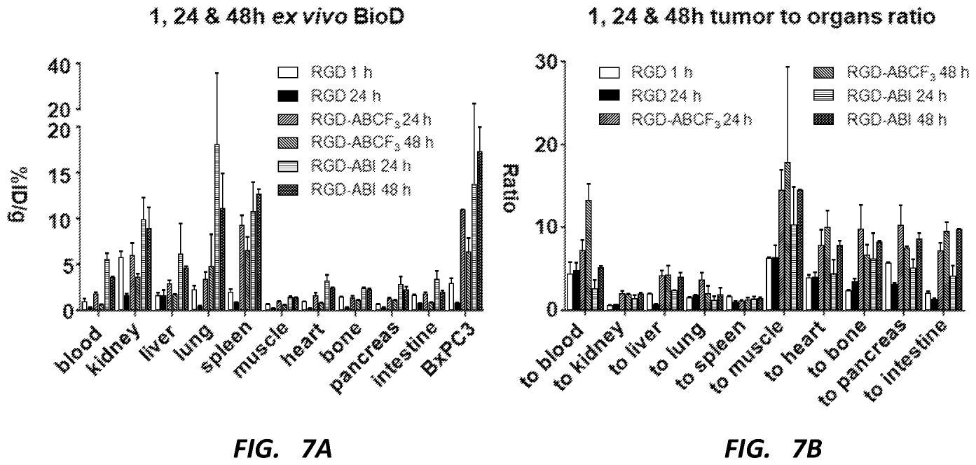

FIGS. 7A and 7B show results comparing ¹¹¹In labeled RGD-ABCF3, RGD and RGD-ABI in mice bearing BxPC3 xenograft: (FIG. 7A) biodistribution, and (FIG. 7B) tumor/nontumor ratios. In the xenografted pancreatic tumor mouse model, compared to integrin α_{V}β3-specific RGD, the ABCF3 incorporated one showed ˜8× enhanced tumor uptake at 24 h post-injection time points; and importantly, in addition to increased tumor uptake, incorporating ABCF3 also improved tumor to nontumor ratios, especially, tumor/kidney, as the kidney would be the dose limiting organ for this RGD peptide. On the other hand, compared to RGD-ABCF3, ABI incorporated RGD showed increase tumor uptake, however its non-tumor uptakes also increased significantly and resulted in decreased tumor/nontumor ratios, especially tumor/blood ratio, because high blood uptake frequently brought concerns on radiotoxicity on red marrow—another typical dose limiting organ for radionuclide therapy.

### b. ¹¹¹In Labeled RGD-ABCF3, RGD and RGD-ABI in Mice Bearing CT26 Allograft

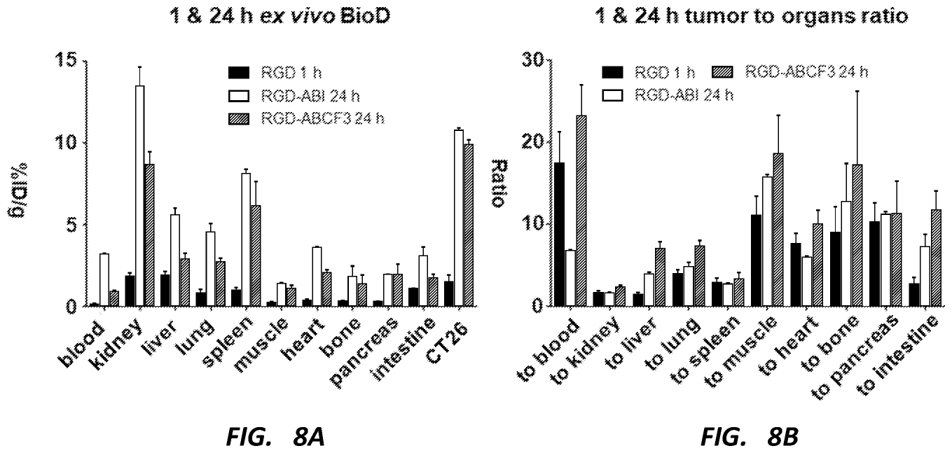

FIGS. 8A and 8B show results comparing ¹¹¹In labeled RGD-ABCF3, RGD and RGD-ABI in mice bearing the CT26 (colorectal cancer) allograft: (FIG. 8A) biodistribution, and (FIG. 8B) tumor/non-tumor ratios. Similarly, incorporating ABCF3 increased tumor uptake ˜5× and also tumor/nontumor ratios; while, incorporating ABI (relative to ABCF3) slightly increased tumor uptake but also resulted in unfavorable tumor/nontumor ratios.

### c. ⁶⁴Cu Labeled PSMA617-ABCF3 and PSMA617 in Mice Bearing PSMA⁺ PC3pip and PSMA-PC3 Xenograft

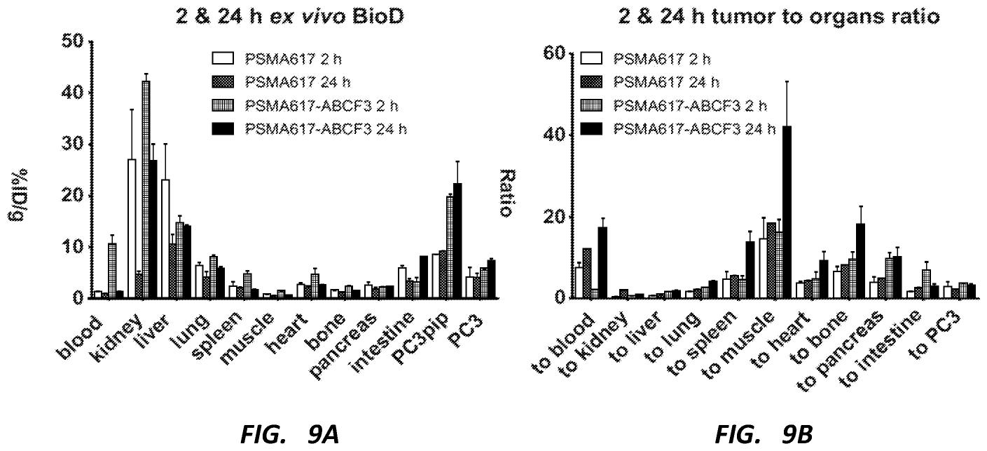

Besides α_{V}β3-specific RGD, ABCF3 was also incorporated into PSMA-specific PSMA-617. FIGS. 9A and 9B show results comparing ⁶⁴Cu labeled PSMA-617-ABCF3 and PSMA617 in mice bearing PSMA⁺ PC3pip and PSMA-PC3 xenograft: (FIG. 9A) biodistribution, and (FIG. 9B) tumor/nontumor ratios.

PC3pip overexpresses PSMA, while PC3 is PSMA negative and does not express PSMA. As shown in FIG. 9, attaching ABCF3 into PSMA617 not only increased its specific tumor uptake >2×, but also enhance tumor/nontumor ratios, especially at late time point (24 h).

### d. ¹¹¹In Labeled FRGD-ABCF3 and FRGD in Mice Bearing BxPC3 Xenograft

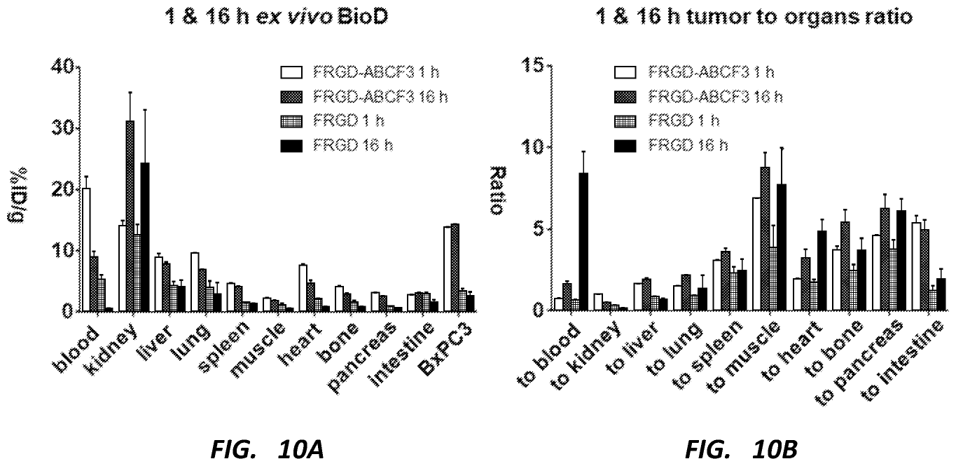

ABCF3 was also attached to α_{V}β6-specific FRGD peptide, the resulting FRGD-ABCF3 was then radiolabeled with ¹¹¹In, and then compared to ¹¹¹In-FRGD in mice bearing BxPC3 xenograft. FIGS. 10A and 10B show results of this comparison: (FIG. 10A) biodistribution, and (FIG. 10B) tumor/non-tumor ratios. The FRGD-ABCF3 showed ˜5× tumor uptakes at both 1 h and 16 h post-injection time points. Except tumor/blood ratio at 16 h, the tumor/nontumor ratios of FRGD-ABCF3 were higher than those of FRGD; especially, tumor/kidney ratio increased ˜3× after ABCF3 was attached. Kidney uptake was very high and would possibly be the dose limiting organ if without ABCF3.

### e. In Vivo Performance of SFLAP3 Incorporated with Various ABX in Mice Bearing BxPC3 Xenograft

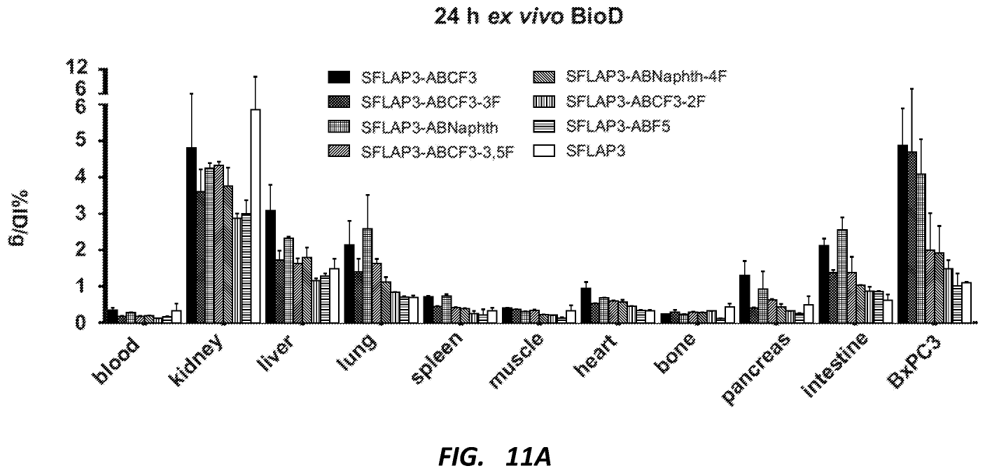

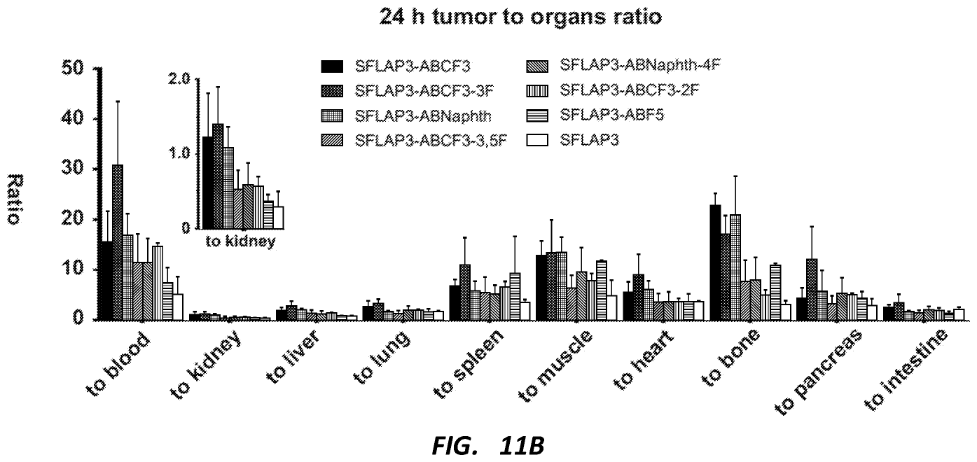

FIGS. 11A and 11B show results comparing in vivo performance of SFLAP3 incorporated with various ABX in mice bearing BxPC3 xenograft (24 h): (FIG. 11A) biodistribution, and (FIG. 11B) tumor/nontumor ratios. A comparison of SFLAP3 self with SFLAP3-ABCF3-2F, SFLAP3-ABNaphth-4F and SFLAP3-ABF5, revealed that there were no significant tumor-uptake increase if the albumin protein binding of ABX was weaker than SFLAP3-ABCF3-2F. This was followed by investigation of the in vivo performance of other ABX (ABCF3, ABCF3-3F, ABNaphth and ABCF3-3F) attached to the SFLAP3 ligand. The results suggested that incorporating ABCF3-3F into the SFLAP3 peptide resulted in the best in vivo performance among all the tested ABX. Additional benefit for using ABX with fluorine (F) on aromatic ring, is the capability on preparing 18F radiolabeled ligand for PET imaging, by replacing the non-radioactive 19F with radioactive 18F.

### III. Discussion

Integrins are transmembrane proteins (receptors) that facilitate cell-extracellular matrix (ECM) adhesion. Activated by ligand binding, integrins are involved in signal transduction pathways and mediate cell survival, differentiation, gene transcription, and apoptosis. Integrins are composed of two subunits (α and β subunits) and functionalize as heterodimers with 24 distinct assemblies. A wide variety of integrins contribute to tumor progression. Integrin α_{V}β₃ receptors (the first characterized α_{V} integrins), bind with fibronectin and vitronectin via Arg-Gly-Asp (RGD) tripeptide motif regulating angiogenesis. Integrin α_{V}β₃ is preferentially overexpressed in glioblastoma, melanoma, breast, prostate, pancreatic cancer cells, but nearly undetectable in most adult epithelia making it a fundamental hallmark of cancer biology. Therefore, inhibition of integrin α_{V}β₃ has been investigated for cancer antiangiogenic therapy. Cilengitide, a cyclized Arg-Gly-Glu (RGD)-containing pentapeptide, could selectively bind cancer cells expressing α_{V}β₃ integrins. Despite demonstrating good tolerance and potent therapeutic efficacy in phase II studies, Cilengitide failed in phase III trials; and one of the reasons was rapid blood clearance, subsequently limiting tumor accumulation. Therefore, the inventors have developed ABCF3 incorporated to cyclo(RGD) to overcome its rapid blood clearance.

Integrin α_{V}β₆ is another important member of integrin family, and it is also an epithelial receptor and upregulated in a variety of cancers including oral squamous cell carcinoma, intestinal gastric carcinoma, nonsmall cell lung carcinoma, ovarian cancer and pancreatic ductal adenocarcinoma; but it has not (or seldom) found in normal epithelium. Utilizing phage-displayed peptide library technology, several peptides with very high α_{V}β₆ specificity have been screened out, including A20FMDV2, SFLAP3, TP H2009.1, etc. All those peptides contain a similar RGDLXXL substructure that is essential for α_{V}β₆-specific binding. A number of α_{V}β₆ targeted radioligands have undergone clinical trials for PET imaging in various diseases. Despite the results encouraged further clinical trials, pharmacokinetic improvements on those radioligands are highly desirable based on the obtained results. Therefore, the inventors have incorporated albumin binder into a α_{V}β₆-specific peptide to improve tumor uptake and also to enhance tumor/nontumor ratios (contrast), particularly on tumor/kidney ratio, because kidney uptake brought the highest background for pancreatic cancer imaging.

Computational-assisted structure optimization was performed on the α_{V}β₆-specific A20FMDV2 peptide, and a very rigid mimic β-sheet structure ligand (FRGD) was successfully identified with high binding affinity (IC₅₀: ˜0.26 nM). In addition to high avidity to α_{V}β₆, this cyclized FRGD peptide also showed great selectivity over other integrins, for example, its IC₅₀ to α_{V}β₃ is ˜640 nM. However, the further in vivo evaluation results showed relatively low tumor uptake and high non-tumor uptakes, which limits its further clinical translation. It was observed that (¹¹¹In)-labeled FRGD-ABCF3 showed ˜5× higher tumor uptake at both 1 h and 16 h when compared to (¹¹¹In)FRGD that does not contain albumin binder. More importantly, at early post-injection time point (1 h), (¹¹¹In) FRGD-ABCF3 showed better (or at least comparable) tumor/non-rumor ratios when compared to those observed with (¹¹¹In)FRGD. It is believed that the significantly improved tumor/kidney, tumor/muscle, and tumor/liver uptake would greatly facilitate the PET imaging of pancreatic tumor (in which kidney and muscle are considered as background), and liver metastasis. In addition to PET imaging, the increased tumor uptake and improved tumor/nontumor ratios will also benefit FRGD-ABCF3's application in radionuclide therapy.

### Definitions

“Theranostics” is the systematic integration of targeted diagnostics and therapeutics. The term “radiotheranostics” refers to the use of radionuclides for the paired imaging and therapy agents.

The term “alkyl” refers to a straight or branched hydrocarbon. For non-limiting examples, an alkyl group can have 1 to 4 carbon atoms (i.e, C₁-C₄ alkyl or C₁₋₄ alkyl) or 1 to 3 carbon atoms (i.e., C₁-C₃ alkyl or C₁₋₃ alkyl). Specific examples of alkyl groups include methyl, ethyl, propyl, isopropyl, n-butyl, sec-butyl, tertiary butyl, and isobutyl groups.

The term “halogen” or “halo” refers to Fluorine (F), Chlorine (CI), Bromine (Br), Iodine (I).

The term “fluoroalkyl” refers to refers to a straight or branched alkyl group in which one or more hydrogen atoms of the alkyl group is replaced with a fluorine (F) atom. The alkyl portion of a fluoroalkyl group can have, for instance, 1 to 6 carbon atoms (i.e., C₁-C₆ fluoroalkyl), 1 to 4 carbon atoms (i.e., C₁-C₄ fluoroalkyl), or 1 to 3 carbon atoms (i.e., C₁-C₃ fluoroalkyl). Non-limiting examples of suitable fluoroalkyl groups include, but are not limited to, trifluoromethyl (—CF₃), difluoromethyl (—CHF₂), fluoromethyl (—CFH₂), 2-fluoroethyl (—CH₂CH₂F), 2-fluoropropyl (—CH₂CHF₂), 2,2,2-trifluoroetheyl (—CH₂CF₃), 1,1-difluoroethyl (—CF₂CH₃), 2-fluoropropyl (—CH₂CHFCH₃), 1,1-difluoropropyl (—CF₂CH₂CH₃), 2,2-difluoropropyl (—CH₂CF₂CH₃), 3,3-difluoropropyl (—CH₂CH₂CHF₂), 3,3,3-trifluoropropyl (—CH₂CH₂CHF₃), 1,1-difluorobutyl (—CF₂CH₂CH₂CH₃), perfluoroethyl (—CF₂CF₃), perfluoropropyl (—CF₂CF₂CF₃), 1,1,2,2,3,3-hexafluorobutyl (—CF₂—CF₂CF₂CH₃), perfluorobutyl (—CF₂CF₂CF₂CF₃), 1,1,1,3,3,3-hexafluoropropan-2-yl (—CH₂(CF₃)₂) groups, and the like. A “perfluoro” alkyl group refers to an alkyl group in which all hydrogen atoms have been replaced with fluorine atoms, such as in trifluoromethyl and pentafluoroethyl groups.

Similar to fluoroalkyl, the term “haloalkyl” refers to refers to a straight or branched alkyl group in which one or more hydrogen atoms of the alkyl group is replaced with a halogen atom, such as a fluorine (F) atom.

The group “SF₂” refers to a difluoro sulfane group, “SF₂Cl” refers to a chlorodifluoro sulfane group, “SF₅” refers to a pentafluoro sulfane group, and “SF₄Cl” refers to a chlorotetrafluoro sulfane group, each depicted, respectively, below.

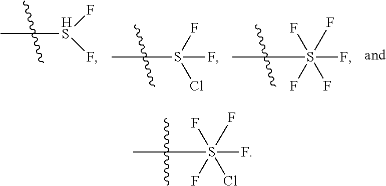

Appearances of a wavy line [symbol] over a straight line in chemical structures indicates a bond through which an atom or chemical group is bound to another atom or chemical group in the structure, definition, or other context in which it is shown.

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “Claims 2-10” or “C₂-C₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

The acronym “RGD” refers to the tripeptide of Arginine-Glycine-Aspartate.

An “amine protecting group” is a chemical group or moiety introduced to a molecule to modify an amine for chemoselectivity in a subsequent chemical reaction or multiple reactions in a multistep organic synthesis. Amine protecting groups may be selected from the group of methyl carbamate, 9-fluorenylmethyl carbamate (Fmoc), 2,2,2,-trichlorethyl carbamate (Troc), 2-trimethylsilylethyl carbamate (Teoc), t-butyl carbamate (BOC), allyl carbamate (Alloc), benzyl carbanate (Cbz), m-nitrophenyl carbamate, formamide, acetamide, trifluoroacetamide, benzyl (benzylamine), allyl (allylamine), and trityl (tritylamine), 3,5-dimethoxyphenylisoproxycarbonyl (Ddz), 2-(4-biphenyl)isopropoxycarbonyl (Bpoc), 2-nitrophenylsulfenyl (Nps), 2-(4-nitrophenylsulfonyl) ethoxycarbonyl (Nsc), 1,1-Dioxobenzo[b]thiophene-2-ylmethyloxycarbonyl (Bsmoc, (1,1-Dioxonaphtho[1,2-b]thiophene-2-yl)methyloxycarbonyl (α-Nsmoc), (1-(4,4-Dimethyl-2,6-dioxocyclohex-1-ylidene)-3-ethyl) (Dde), 1-(4,4-Dimethyl-2,6-dioxocyclohex-1-ylidene)-3-mehtylbutyl (ivDde), 2-Fluoro-Fmoc (Fmoc(2F)), 2-Monoisooctyl-Fmoc (mio-Fmoc), 2,7-Diisooctyl-Fmoc (dio-Fmoc), Tetrachlorophthaloyl (TCP), 2-[Phenyl(methyl)sulfonio]ethyloxycarbonyl tetrafluoroborate (Pms), Ethanesulfonylethoxycarbonyl (Esc), 2-(4-Sulfophenylsulfonyl)ethoxycarbonyl (Sps), Benzyloxycarbonyl (Z), Allyloxycarbonyl (Alloc), o-Nitrobenzenesulfonyl (oNBS), p-nitrobenzenesulfonyl (pNBS), 2,4-Dinitrobenzenesulfonyl (dNBS), Benzothiazole-2-sulfonyl (Bts), 2-Nitrophenylsulfanyl (Nps), Dithiasuccinoyl (Dts), p-Nitrobenzyloxycarbonyl (pNZ), Propargyloxycarbonyl (Poc), 2-(3,4-Methylenedioxy-6-nitrophenyl)propyloxycarbonyl (MNPPOC), 9-(4-Bromophenyl)-9-fluorenyl (BrPhF), Azidomethyloxycarbonyl (Azoc), o-Nitrobenzyloxycarbonyl (oNZ), 4-Nitroveratryloxycarbonyl (NVOC), 4-nitroveratryloxycarbonyl (NPPOC), and hexafluoroacetone (HFA) protecting groups.

Amide protecting groups for amines include formamide, acetamide, and trifluoroacetamide protecting groups. Sulfonamide protecting groups for amines include p-toluenesulfonyl (Ts), trifluoromethanesulfonyl, trimethylsilylethanesulfonamide (SES), and tert-butylsulfonyl (Bus) protecting groups.

What is claimed:

1. A compound of Formula (I): 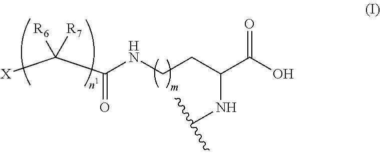
wherein:
X is selected from the group of: 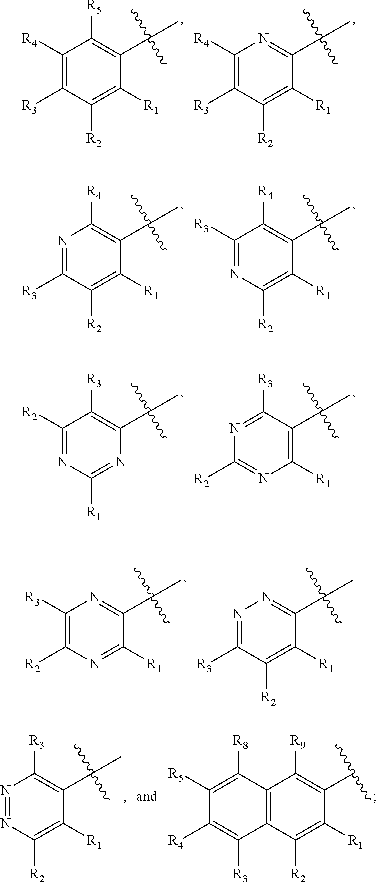
R₁, R₂, R₃, R₄, R₅, and when present, R₈ and R₉ are each independently selected from the group of H, F, Cl, Br, I, SF₃, SF₂Cl, SF₅, SF₄Cl, C₁-C₆ straight or branched alkyl, C₁-C₆ straight or branched fluoroalkyl, C₁-C₆ straight or branched fluorinated alkoxy, and isotopes thereof, or a substituent selected from the group of: 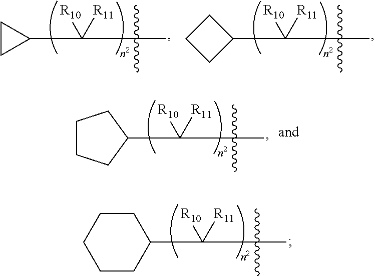
R₆ and R₇ are in each instance independently selected from H, F, and isotopes thereof, or combined in an oxo group;
R₁₀ and R₁₁ are in each instance independently selected from H, F, and isotopes thereof;
n¹ is an integer selected from the group of 1, 2, 3, 4, 5, and 6;
n² is an integer selected from the group of 1, 2, 3, and 4; and
m is an integer selected from the group of 1, 2, 3, 4, 5, 6, 7, and 8;
with the proviso that when X is a substituted benzyl group at least one of R₁, R₂, R₃, R₄, and R₅ is selected from the group of F, SF₃, SF₅, C₁-C₆ straight or branched fluoroalkyl, and isotopes thereof or a substituent selected from the group of:

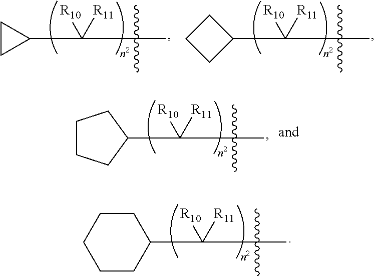

2. A compound of the formula (II): 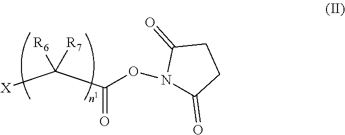
wherein X, R₁, R₂, R₃, R₄, R₅, R₆, R₇, R₈, R₉, R₁₀, R₁₁, n¹, and n² are as defined in claim 1.

3. The compound of any of claims 1 and 2, wherein X is the group:

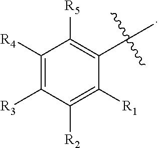

4. The compound of any of claims 1 and 2, wherein X is selected from the group of:

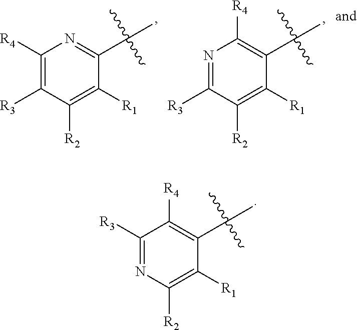

5. The compound of any of claims 1 and 2, wherein X is selected from the group of:

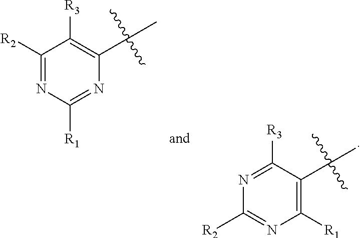

6. The compound of any of claims 1 and 2, wherein X is the group:

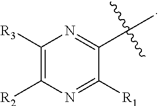

7. The compound of any of claims 1 and 2, wherein X is selected from the group of:

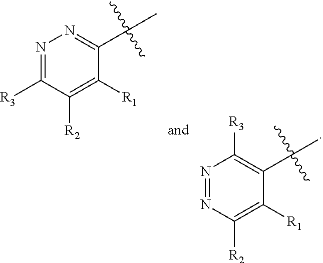

8. The compound of any of claims 1 and 2, wherein X is

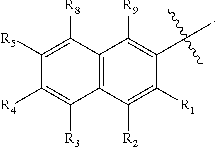

9. The compound of any of claims 1-8, wherein R₁, R₂, R₃, R₄, R₅, and when present, R₈ and R₉ are each independently selected from the group of H, F, Cl, Br, I, SF₃, SF₂Cl, SF₅, SF₄Cl, C₁-C₆ straight or branched alkyl, C₁-C₄ straight or branched fluoroalkyl, C₁-C₄ straight or branched fluorinated alkoxy, and isotopes thereof, or a substituent selected from the group of: 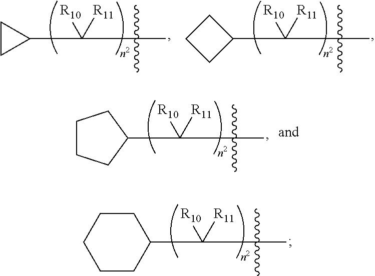
R₁₀ and R₁₁ are in each instance independently selected from H, F, and isotopes thereof; and
n² is an integer selected from the group of 1, 2, 3, and 4.

10. The compound of any of claims 1-8, wherein R₁, R₂, R₃, R₄, R₅, and when present, R₈ and R₉ are each independently selected from the group of H, F, Cl, Br, I, C₁-C₄ straight or branched alkyl, C₁-C₄ straight or branched fluoroalkyl, C₁-C₄ straight or branched fluorinated alkoxy, and isotopes thereof, or a substituent selected from the group of: 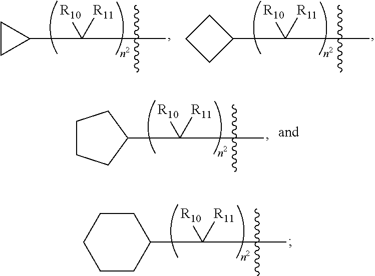
R₁₀ and R₁₁ are in each instance independently selected from H, F, and isotopes thereof; and
n² is an integer selected from the group of 1, 2, and 3.

11. The compound of any of claims 1-10, wherein R₁, R₂, R₃, R₄, R₅, and when present, R₈ and R₉ are each independently selected from the group of H, F, Cl, Br, I, C₁-C₄ straight or branched alkyl, C₁-C₄ straight or branched fluoroalkyl, C₁-C₄ straight or branched fluorinated alkoxy, and isotopes thereof;
with the proviso that at least one of R₁, R₂, R₃, R₄, R₅, and if present, R₈ and R₉ is selected from the group of F and C₁-C₄ straight or branched fluoroalkyl, C₁-C₄ straight or branched fluorinated alkoxy, and isotopes thereof.

12. The compound of any of claims 1-11, wherein at least one of R₁, R₂, R₃, R₄, R₅, and when present, R₈ and R₉ is F.

13. The compound of any of claims 1-12, wherein at least two of R₁, R₂, R₃, R₄, R₅, and when present, R₈ and R₉ are F.

14. The compound of any of claims 1-13, wherein at least three of R₁, R₂, R₃, R₄, R₅, and when present, R₈ and R₉ are F.

15. The compound of any of claims 1-14, wherein at least one of R₁, R₂, R₃, R₄, R₅, and when present, R₈ and R₉ is selected from the group of:
a) —CH₂F, —CHF₂, —CF₃;
b) —CHFCH₃, —CF₂CH₃, —CH₂CHF₂, —CH₂—CF₃, —CH₂CH₂F, —CHF—CF₃, —CF₂CF₃;
c) —CHFCH₂CH₃, —CF₂CH₂CH₃, —CH₂CHFCH₃, —CH₂CF₂CH₃, —CH₂CH₂CH₂F, —CH₂CH₂CHF₂, —CH₂CH₂CF₃, —CH₂CF₂CF₂, —CH₂CF₂CF₃, —CHFCH₂CHF₂, —CHFCF₂CHF₂, —CF₂CH₂CHF₂, —CF₂CH₂CF₃, —CF₂CF₂CHF₂, —CF₂CF₂CF₃;
d) —CF₂CH(CH₃)₂, —CF₂CH₂CH₂CH₃, —CH₂CF₂CH₂CH₃, —CH₂CH₂CF₂CH₃, —CH₂CH₂CH₂CH₂F, —CH₂CH₂CH₂CHF₂, —CH₂CH₂CH₂CF₃, —CH₂CF(CH₃)₂;

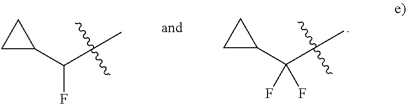 [SMILES: *C(F)C1CC1 ; *C(F)(F)C1CC1]

16. The compound of any of claims 1-15, wherein at least one of R₁, R₂, R₃, R₄, R₅, and when present, R₈ and R₉ is selected from the group of —CH₂F, —CHF₂, and —CF₃.

17. The compound of any of claims 1-15, wherein at least one of R₁, R₂, R₃, R₄, R₅, and when present, R₈ and R₉ is selected from the group of —CHFCH₃, —CF₂CH₃, —CH₂CHF₂, —CH₂—CF₃, —CH₂—CF₃, —CH₂CH₂F, —CHF—CF₃, and —CF₂CF₃.

18. The compound of any of claims 1-15, wherein at least one of R₁, R₂, R₃, R₄, R₅, and when present, R₈ and R₉ is selected from the group of —CHFCH₂CH₃, —CF₂CH₂CH₃, —CH₂CHFCH₃, —CH₂CF₂CH₃, —CH₂CH₂CH₂F, —CH₂CH₂CHF₂, —CH₂CH₂CF₃, —CH₂CF₂CF₂, —CH₂CF₂CF₃, —CHFCH₂CHF₂, —CHFCF₂CHF₂, —CF₂CH₂CHF₂, —CF₂CH₂CF₃, —CF₂CF₂CHF₂, and —CF₂CF₂CF₃.

19. The compound of any of claims 1-15, wherein at least one of R₁, R₂, R₃, R₄, R₅, and when present, R₈ and R₉ is selected from the group of —CF₂CH(CH₃)₂, —CF₂CH₂CH₂CH₃, —CH₂CF₂CH₂CH₃, —CH₂CH₂CF₂CH₃, —CH₂CH₂CH₂CH₂F, —CH₂CH₂CH₂CHF₂, —CH₂CH₂CH₂CF₃, and —CH₂CF(CH₃)₂.

20. The compound of any of claims 1-15, wherein at least one of R₁, R₂, R₃, R₄, R₅, and when present, R₈ and R₉ is selected from the group of:

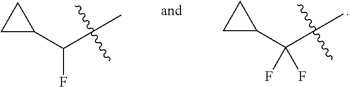 [SMILES: *C(F)C1CC1 ; *C(F)(F)C1CC1]

21. The compound of any of claims 1-15, wherein at least one of R₁, R₂, R₃, R₄, R₅, and when present, R₈ and R₉ is selected from the group of: SF₃, SF₂Cl, SF₅, and SF₄Cl.

22. The compound of any of claims 1-15, wherein at least one of R₁, R₂, R₃, R₄, R₅, and when present, R₈ and R₉ is —OCF₃.

23. The compound of any of claims 1-22, wherein n¹ is an integer selected from the group of 1, 2, 3, 4, and 5.

24. The compound of any of claims 1-23, wherein n¹ is an integer selected from the group of 1, 2, 3, and 4.

25. The compound of any of claims 1-23, wherein n¹ is an integer selected from the group of 1, 2, and 3.

26. The compound of any of claims 1-23, wherein n¹ is an integer selected from the group of 4, 5, and 6.

27. The compound of any of claims 1-26, wherein, in each appearance, R₆ and R₇ are hydrogen.

28. The compound of any of claims 1-26, wherein in at least one appearance R₆ and R₇ are combined in an oxo group.

29. The compound of any of claims 1 and 3-28, wherein m, when present, is an integer selected from the group of 1, 2, 3, and 4.

30. The compound of any of claims 1 and 3-28, wherein m, when present, is an integer selected from the group of 5, 6, 7, and 8.

31. The compound of any of claims 1 and 3-28, wherein m, when present, is an integer selected from the group of 3, 4, 5, and 6.

32. The compound of any of claims 1 and 3-31, wherein the compound binds serum albumin through non-covalent interaction of X with a binding site of serum albumin.

33. The compound of any of claims 1 and 3-32, wherein the compound binds serum albumin through non-covalent interaction of the carboxyl group of Formula (I) with a binding site of serum albumin.

34. The compound of claim 2, having Formula (IIa):

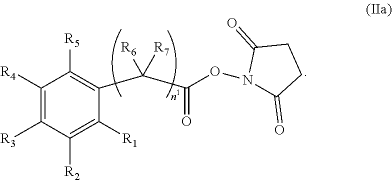

35. The compound of claim 2, having Formula (IIb):

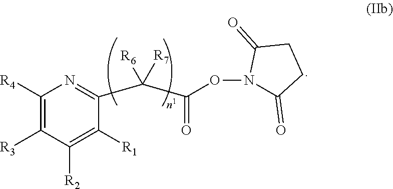

36. The compound of claim 2, having Formula (IIc):

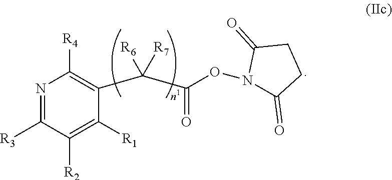

37. The compound of claim 2, having Formula (IId):

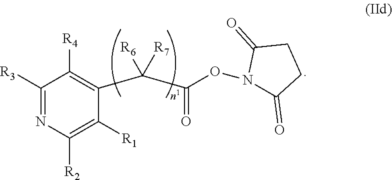

38. The compound of claim 2, having Formula (IIe):

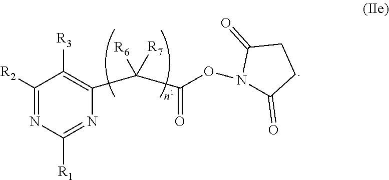

39. The compound of claim 2, having Formula (IIf):

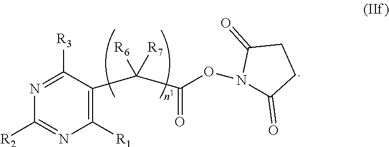

40. The compound of claim 2, having Formula (IIg):

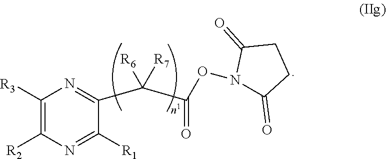

41. The compound of claim 2, having Formula (IIh):

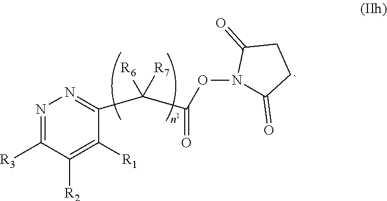

42. The compound of claim 2, having Formula (IIi):

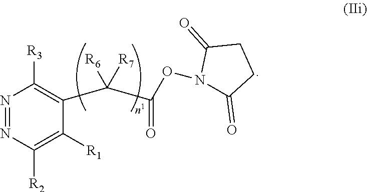

43. The compound of claim 2, having Formula (IIj):

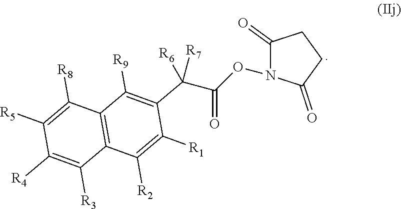

44. The compound of any of claims 1, 3-26, 28, and 31, wherein m, when present, is 3.

45. The compound of any of claims 1-44, wherein at least one of R₁, R₂, R₃, R₄, R₅, R₆, R₇, R₈ (if present), R₉ (if present), R₁₀, and R₁₁ comprises ¹⁸F or a functional group containing ¹⁸F.

46. The compound

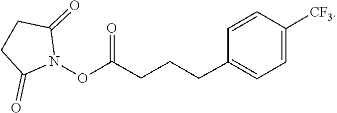 [SMILES: O=C(CCCc1ccc(C(F)(F)F)cc1)ON1C(=O)CCC1=O]

47. A composition comprising a product of a reaction in which a therapeutic agent is complexed with the compound of any of claims 1-45.

48. The composition of claim 47, wherein the therapeutic agent is non-covalently bound to the compound.

49. The composition of claim 47, wherein the therapeutic agent is covalently linked to the compound.

50. The composition of claim 49, wherein the therapeutic agent is linked to the compound via a linker.

51. The composition of any of claims 47-50, wherein the therapeutic agent is an anti-cancer drug or an anti-inflammatory drug.

52. The composition of claim 51, wherein the anti-cancer drug is selected from the group consisting of alkylating drugs, anthracyclines, cytotoxic antibiotics, anti-metabolites, vinca alkaloids, platinum-based anti-neoplastic agents, taxanes, epothilones, histone deacetylase inhibitors, topoisomerase I inhibitors, topoisomerase II inhibitors, kinase inhibitors, retinoids, nucleotide analogs, and precursor analogs.

53. The composition of claim 51, wherein the anti-cancer drug is selected from the group consisting of actinomycin, all-trans retinoic acid, alitretinoin, azacytidine, azathioprine, bexarotene, leomycin, bortezomib, carboplatin, capecitabine, cisplatin, chlorambucil, cyclophosphamide, cytarabine, daunorubicin, dacarbazine, docetaxel, doxifluridine, doxorubicin, epirubicin, epothilone, erlotnib, etoposide, fluorouracil, gefitnib, gemcitabine, hydroxyurea, idarubicin, imatinib, irinotecan, mechlorethamine, mercaptopurine, methotrexate, melphalan, mitoxantrone, nitrosourea, oxaliplatin, paclitaxel, pemetrexed, tafluposide, temozolomide, teniposide, tioguanine, topotecan, tretinoin, valrubicin, vemurafenib, vinblastine, vincristine, vindesine, vinorelbine, vismodegib, vorinostat, cyclophosphamide, ifospfamide, busulfan, lomustine, carmustine, chlormethine, altretamine, estramustine, treosulfan, thiotepa, mitobronitol, aclarubicin, idarubicin, dactinomycin, mitomycin, pentostatin, fludarabine, cladribine, raltitrexed, tegafur, amsacrine, asparaginase, trastuzumab, and derivatives thereof.

54. A composition comprising a product of a reaction in which a targeting ligand is complexed with the compound of any of claims 1-45.

55. The composition of claim 54, wherein the targeting ligand is non-covalently bound to the compound.

56. The composition of claim 54, wherein the targeting ligand is covalently linked to the compound.

57. The composition of claim 56, wherein the targeting ligand is linked to the compound via a linker.

58. The composition of any of claims 54-57, wherein the targeting ligand is selected from the group consisting of proteins, polysaccharides, nucleic acids, peptides, aptamers, and small molecules.

59. The composition of claim 58, wherein the targeting ligand is a peptide selected from the group consisting of arginylglycyclaspartic acid (RGD), galacto-RGD₂, P-RGD, RGD₂, P-RGD₂, 2G-RGD₂, 2P-RGD₂, 3G-RGD₂, 3P-RGD₂, 3P-RGK₂, RGD₄, 6G-RGD₄, 6P-RGD₄, FAPI, AE105, NT20.3, A20FMDV2, Pentixafor, SFLAP3, JR11, DOTATATE and PSMA-617.

60. The composition of any of claims 54-59, wherein the targeting ligand is targeted to a receptor that is overexpressed in a tissue affected by a disease.

61. The composition of claim 60, wherein the disease is cancer or an inflammatory disease.

62. The composition of any of claim 60 and 61, wherein the receptor is an integrin, a lectin, or a cytokine.

63. The composition of claim 62, wherein the integrin is selected from the group consisting of α_{V}β₃, α_{V}β₄, α_{V}β₅, α_{V}β₆, α₅β₁, α_{V}β1, α_{V}β₈, α8β₁, α_{IIb}β₃, α₄β₁, α₉β₁, α₄β₇, α_{E}β₂, α_{L}β₂, α_{M}β₂, α_{X}β₂, α_{D}β₂, α₁β₁, α₂β₁, α₁₀β₁, α₁₁β₁, α₃β₁, α₆β₁, α₇β₁, and α₆β₄.

63. A composition comprising a product of a reaction in which a radionuclide chelator is complexed with the compound of any of claims 1-45 or the composition of any one of claims 47-63.

64. The composition of claim 63, wherein the radionuclide chelator is non-covalently bound to the compound.

65. The composition of claim 63, wherein the radionuclide chelator is covalently linked to the compound.

66. The composition of claim 65 wherein the radionuclide chelator is linked to the compound via a linker.

67. The composition of any of claims 63-66, wherein the radionuclide chelator is selected from the group consisting of 2,2′,2″,2″′-(1,4,7,10-tetraazacyclododecane-1,4,7,10-tetrayl)tetraacetic acid (DOTA), Hexahydro-1H-1,4,7-triazonine-1,4,7-triacetic acid (NOTA), 1,4,7-Tris(phosphonomethyl)-1,4,7-triazacyclononane (NOTP), ((1,4,7-triazonane-1,4,7-triyl)tris(methylene))tris(phosphinic acid) (TRAP), N′-[5-[[4-[[5-(acetylhydroxyamino)pentyl]amino]-1,4-dioxobutyl]hydroxyamino]pentyl]-N-(5-aminopentyl)-N-hydroxy-butanediamide (DFO), 2,2′,2″,2″′-((((carboxymethyl)azanediyl)bis(ethane-2,1-diyl))bis(azanetriyl))tetraacetic acid (DTPA), 3,12-bis(carboxymethyl)-6,9-dioxa-3,12-diazatetradecanedioic acid (EGTA), 2,2′,2″,2″′-(ethane-1,2-diylbis(azanetriyl))tetraacetic acid (EDTA), 7-[2-[bis(carboxymethyl)amino]-3-(4-nitrophenyl)propyl]hexahydro-1H-1,4,7-Triazonine-1,4(5H)-diacetic acid (C-NETA), 2-(4,7-bis(carboxymethyl)-1,4,7-triazonan-1-yl)pentanedioic acid (NODAGA), 2-(4,7,10-tris(carboxymethyl)-1,4,7,10-tetraazacyclododecan-1-yl)-pentanedioic acid (DOTAGA), 1,4,7-triazacydononane-1-[methyl(2-carboxyethyl)-phosphinic acid]-4,7-bis[methyl(2-hydroxymethyl)phosphinic acid] (NOPO), 3,6,9,15-tetraazabicyclo[9,3,1]pentadeca-1 (15), 11,13-triene-3,6,9-triacetic acid (PCTA), N,N″-bis[2-hydroxy-5-(carboxyethyl)-benzyl]ethylenediamine-N,N″-diacetic acid (HBED-CC), N,N′-bis(2,2-dimethyl-2-mercaptoethyl)ethylenediamine-N,N′-diacetic acid (6SS), 1-(4-carboxymethoxybenzyl)-N-N′-bis[(2-mercapto-2,2-dimethyl)ethyl]-1,2-ethylenediamine-N,N′-diacetic acid (B6SS), N,N′-dipyridoxylethylenediamine-N,N′-diacetic acid (PLED), 1,1,1-Tris-(aminomethyl)ethane (TAME), nitrilotrimethylphosphonic acid (NTP), 2-BAPEN, 2,2′,2″,2″′-(1,4,8,11-tetraazacyclotetradecane-1,4,8,11-tetrayl)tetraacetic acid (TOTA), and derivatives thereof.

68. The composition of any of claims 63-67, wherein the radionuclide chelator is chelated to a radionuclide.

69. The composition of claim 68, wherein the radionuclide is selected from the group consisting of ¹⁸F, ¹¹C, ¹⁴C , ⁵¹Cr, ¹³N, ⁷⁵Br, ⁷⁶Br, ¹²³I, ¹²⁵I, ¹²³I, ¹²⁴I, ¹³¹I, ⁴⁸V, ⁵⁷Co, ⁵⁸Co, ⁵⁵Co, ⁸²Rb, ^{94m}Tc, ¹³³Xe, or ⁶⁸Ga, ¹⁵O, ²⁰¹Tl, ¹⁸⁸Rh, ⁴⁷Ca, ¹⁶⁹Er, ³²P, ²²³Ra, ³H, ^{81m}Kr, ²²Na, and ²⁴Na, ¹⁷⁷Lu, ⁸⁶Y, ⁹⁰Y, ⁸⁹Zr, ⁴⁷Sc, ⁴⁴Sc, ²¹³Bi, ^{99m}Tc, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ^{113m}In, ¹⁴⁸Gd, ⁵²Fe, ⁵⁵Fe, ⁵⁹Fe, ²²⁵Ac, ²¹²Bi, ²¹²Pb, ²¹¹At, ⁴⁵Ti, ⁶⁰Cu, ⁶¹Cu, ⁶⁷Cu, and ⁶⁴Cu.

70. The composition of any of claims 54-59 and 63-69, wherein the targeting ligand is targeted to a drug or a metabolite thereof.

71. The composition of any of claims 60-69, wherein a binding affinity of the targeting ligand for the receptor is less than a binding affinity of any part of the compound for serum albumin.

72. The composition of claim 70, wherein a binding affinity of the targeting ligand for the drug is less than a binding affinity of any part of the compound for serum albumin.

73. The composition of any of claims 50, 57, and 66, wherein the linker has the Formula (L1):

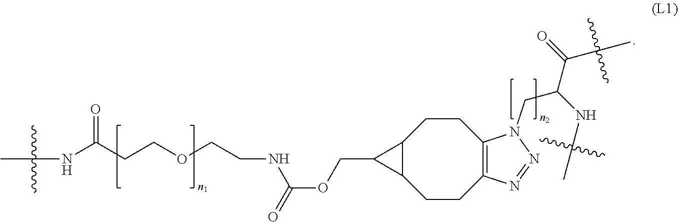

74. The composition of claim 73, wherein n₁ is an integer selected from the group of 1-30 and n₂ is an integer selected from the group of 2-10.

75. The composition of claim 74, wherein n₁ is an integer selected from the group of 2-20 and n₂ is an integer selected from the group of 2-8.

76. The composition of claim 75, wherein n₁ is an integer selected from the group of 2-20 and n₂ is an integer selected from the group of 2-8.

77. The composition of any of claims 50, 57, or 66, wherein the linker has the Formula (L2):

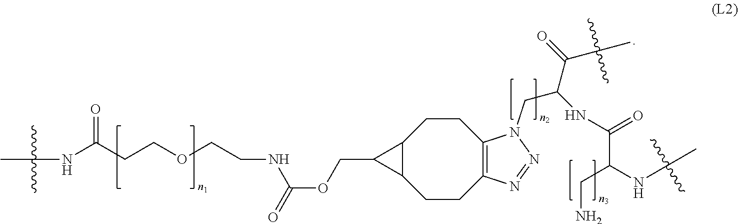

78. The composition of claim 77, wherein n₁ is an integer selected from the group of 1-30, n₂ is an integer selected from the group of 2-10, and n3 is an integer selected from the group of 1-10.

79. The composition of claim 78, wherein n₁ is an integer selected from the group of 2-20, n₂ is an integer selected from the group of 2-8, and n₃ is an integer selected from the group of 2-8.

80. The composition of claim 79, wherein n₁ is an integer selected from the group of 2-15, n₂ is an integer selected from the group of 2-6, and n₃ is an integer selected from the group of 2-6.

81. A method of increasing the circulatory half-life of a drug, comprising complexing the drug with the compound of any of claims 1-45.

82. A method of increasing the therapeutic efficacy of a drug, comprising complexing the drug with the compound of any of claims 1-45.

83. A method of treating a subject, comprising administering a therapeutically effective dose of the composition of any of claims 47-53, 68 and 69.

84. The method of claim 83, wherein the therapeutically effective dose is lower than a minimum therapeutically effective dose of the therapeutic alone.

85. The method of any of claims 83 and 84 wherein the composition is administered intravenously or intramuscularly.

86. A method of treating a subject, comprising:
a) administering the composition of any of claims 63-69 to the subject; and
b) measuring a level of the composition in a sample from the subject.

87. The method of claim 86, wherein the compound is administered intravenously or intramuscularly.
